(19) United States
(12) Patent Application Publication
(10) Pub. No.: US 2023/0399289 A1
(43) Pub. Date: Dec. 14, 2023

(54) ITACONIC ACID DERIVATIVES

(71) Applicant: Sitryx Therapeutics Limited, Oxford (GB)

(72) Inventors: Matthew Colin Thor FYFE, Oxford (GB); Barry John TEOBALD, Oxford (GB)

(73) Assignee: Sitryx Therapeutics Limited, Oxford (GB)

(21) Appl. No.: 18/034,143

(22) PCT Filed: Oct. 29, 2021
(86) PCT No.: PCT/GB2021/052802

§ 371 Date: Apr. 27, 2023

(30) Foreign Application Priority Data
Oct. 29, 2020 (EP) 20204614.0
Oct. 29, 2020 (EP) 20204623.1
Jan. 8, 2021 (EP) 21150656.3
Mar. 8, 2021 (EP) 21161329.4
Jul. 28, 2021 (EP) 21188116.4

(51) Int. Cl. C07C 69/75; C07D 305/08; C07C 69/743; C07C 323/22; A61P 29/00

(52) U.S. Cl. CPC C07C 69/75; C07D 305/08; C07C 69/743; C07C 323/22; A61P 29/00; C07C 2601/16; C07C 2601/04

(57) ABSTRACT

The invention relates to compounds of formula (I) and to their use in treating or preventing an inflammatory disease or a disease associated with an undesirable immune response:

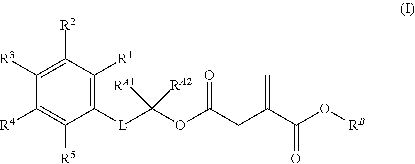

wherein R^{A1}, R^{A2}, R¹ to R⁵, L and R^{B} are as defined herein.

This application is the National Stage of International Application No. PCT/GB2021/052802 filed Oct. 29, 2021, which claims priority to and benefit of European Application Nos. 20204614.0 filed Oct. 29, 2020, 20204623.1 filed Oct. 29, 2020, 21150656.3, filed Jan. 8, 2021, 21161329.4, filed Mar. 8, 2021 and 21188116.4 filed Jul. 28, 2021, each of which is herein incorporated by reference in its entirety.

### FIELD OF THE INVENTION

The present invention relates to compounds and their use in treating or preventing inflammatory diseases or diseases associated with an undesirable immune response, and to related compositions, methods and intermediate compounds.

### BACKGROUND OF THE INVENTION

Chronic inflammatory diseases such as rheumatoid arthritis, systemic lupus erythematosus (SLE), multiple sclerosis, psoriasis, Crohn's disease, ulcerative colitis, uveitis and chronic obstructive pulmonary disease (COPD) represent a significant burden to society because of life-long debilitating illness, increased mortality and high costs for therapy and care (Straub R. H. and Schradin C., 2016). Non-steroidal anti-inflammatory drugs (NSAIDs) are the most widespread medicines employed for treating inflammatory disorders, but these agents do not prevent the progression of the inflammation and only treat the accompanying symptoms. Glucocorticoids are powerful anti-inflammatory agents, making them emergency treatments for acute inflammatory flares, but given longer term these medicines give rise to a plethora of unwanted side-effects and may also be subject to resistance (Straub R. H. and Cutolo M., 2016). Thus, considerable unmet medical need still exists for the treatment of inflammatory disorders and extensive efforts to discover new medicines to alleviate the burden of these diseases is ongoing (Hanke T. et al., 2016).

Dimethyl fumarate (DMF), a diester of the citric acid cycle (CAC) intermediate fumaric acid, is utilised as an oral therapy for treating psoriasis (Brück J. et al., 2018) and multiple sclerosis (Mills E. A. et al., 2018). Importantly, following oral administration, none of this agent is detected in plasma (Dibbert S. et al., 2013), the only drug-related compounds observed being the hydrolysis product monomethyl fumarate (MMF) and glutathione (GSH) conjugates of both the parent (DMF) and metabolite (MMF). DMF's mechanism of action is complex and controversial. This compound's efficacy has been attributed to a multiplicity of different phenomena involving covalent modification of proteins and the conversion of “prodrug” DMF to MMF. In particular, the following pathways have been highlighted as being of relevance to DMF's anti-inflammatory effects: 1) activation of the anti-oxidant, anti-inflammatory, nuclear factor (erythroid-derived 2)-like 2 (NRF2) pathway as a consequence of reaction of the electrophilic α,β-unsaturated ester moiety with nucleophilic cysteine residues on kelch-like ECH-associated protein 1 (KEAP1) (Brennan M. S. et al., 2015); 2) induction of activating transcription factor 3 (ATF3), leading to suppression of pro-inflammatory cytokines interleukin (IL)-6 and IL-8 (Müller S. et al., 2017); 3) inactivation of the glycolytic enzyme glyceraldehyde 3-phosphate dehydrogenase (GAPDH) through succination of its catalytic cysteine residue with a Michael accepting unsaturated ester (Kornberg M. D. et al., 2018; Angiari S. and O'Neill L. A., 2018); 4) inhibition of nuclear factor-kappaB (NF-κB)-driven cytokine production (Gillard G. O. et al., 2015); 5) preventing the association of PKC6 with the costimulatory receptor CD28 to reduce the production of IL-2 and block T-cell activation (Blewett M. M. et al., 2016); 6) reaction of the electrophilic α,β-unsaturated ester with the nucleophilic thiol group of anti-oxidant GSH, impacting cellular responses to oxidative stress (Lehmann J. C. U. et al., 2007); 7) agonism of the hydroxycarboxylic acid receptor 2 (HCA2) by the MMF generated in vivo through DMF hydrolysis (von Glehn F. et al., 2018); 8) allosteric covalent inhibition of the p90 ribosomal S6 kinases (Andersen J. L. et al., 2018); 9) inhibition of the expression and function of hypoxia-inducible factor-1α (HIF-1α) and its target genes, such as IL-8 (Zhao G. et al., 2014); and 10) inhibition of Toll-like receptor (TLR)-induced M1 and K63 ubiquitin chain formation (McGuire V. A. et al., 2016). In general, with the exception of HCA2 agonism (Tang H. et al., 2008), membrane permeable diester DMF tends to exhibit much more profound biological effects in cells compared to its monoester counterpart MMF. However, the lack of systemic exposure of DMF in vivo has led some researchers to assert that MMF is, in fact, the principal active component following oral DMF administration (Mrowietz U. et al., 2018). As such, it is evident that some of the profound biology exerted by DMF in cells is lost because of hydrolysis in vivo to MMF.

Recently, it has been discovered that, during inflammatory macrophage activation, the CAC becomes anaplerotic and is diverted such that the unsaturated diacid itaconic acid, “itaconate”, is generated (Murphy M. P. and O'Neill L. A. J., 2018; O'Neill L. A. J. and Artyomov M. N., 2019; Yu X.-H. et al., 2019). Instead of being hydrated to isocitrate by aconitate hydratase, the CAC intermediate aconitate is decarboxylated by the protein product of immune-responsive gene 1 (IRG1), one of the most highly upregulated genes in macrophages under proinflammatory conditions, subsequently named aconitate decarboxylase 1, to produce itaconic acid (Michelucci A. et al., 2013). This unsaturated diacid is an inhibitor of the bacterial enzyme isocitrate lyase and, as such, it exerts anti-bacterial activity. In addition, itaconic acid has been shown to inhibit the CAC enzyme succinate dehydrogenase (SDH) (Ackermann et al., 1949), leading accordingly to succinate accumulation (Cordes T. et al., 2016). By inhibiting SDH, an enzyme critical for the inflammatory response (E. L. Mills et al., 2016), itaconate ameliorates inflammation in vitro and in vivo during macrophage activation and ischemia-reperfusion injury (Lampropoulou V. et al., 2016).

Like fumaric acid, itaconic acid is an α,β-unsaturated carboxylic acid. As such, it is a Michael acceptor which induces a global electrophilic stress response. In this regard, the itaconic acid diester dimethyl itaconate (DMI), like DMF, produces an anti-inflammatory response, reducing the expression levels of pro-inflammatory cytokines IL-1β, IL-6, IL-12 and IL-18 in lipopolysaccharide (LPS)-stimulated bone marrow-derived macrophages (WO2017/142855A1, incorporated herein by reference). This response appears to be mediated, in part, by NRF2 activation, via alkylation of KEAP1 cysteine residues by the electrophilic α,β-unsaturated ester moiety (Mills E. L. et al., 2018), which enhances the expression of downstream genes with anti-oxidant and anti-inflammatory capacities. Nevertheless, not all of the pronounced immunoregulatory effects engendered by DMI can be attributed to NRF2 activation. In particular, the modulation of IκBζ by DMI is independent of NRF2 and is mediated via upregulation of ATF3, a global negative regulator of immune activation that downregulates various cytokines, such as IL-6 (Bambouskova M. et al., 2018). Moreover, by inhibiting IκBζ protein production, DMI ameliorates IL-17-mediated pathologies, highlighting the therapeutic potential of this regulatory pathway (WO2019/036509A1, incorporated herein by reference). Further highlighting its pharmacologic potential, DMI has recently been reported to 1) demonstrate a protective effect on cerebral ischemia/reperfusion injury, thereby offering potential for the treatment of ischemic stroke (Zhang D. et al., 2019); 2) provide protection from the cardiotoxic effects of doxorubicin (Shan Q. et al., 2019); and 3) protect against lippolysacchride-induced mastitis in mice by activating MAPKs and NRFrf2 while inhibiting NF-κB signaling pathways (Zhao C. et al., 2019). Furthermore, DMI is said to have utility in preventing and treating ulcerative colitis and canceration thereof (CN110731955, Sun Yat-sen University Cancer Center); and has been reported to protect against fungal keratitis by activating the NRF2/HO-1 signalling pathway (Gu L. et al., 2020). Nevertheless, it should be noted that DMI is not metabolised to itaconic acid intracellularly (ElAzzouny M. et al., 2017). Other α,β-unsaturated esters exhibit IL-1β-lowering effects in macrophages by inhibiting the NLRP3 inflammasome (Cocco M. et al., 2017 and 2014), and have been demonstrated to inhibit the TLR4 pathway, leading ultimately to suppression of LPS-induced stimulation of NF-κB, tumour necrosis factor (TNF)-α, IL-1β and nitric oxide release (Zhang S. et al., 2012).

Other itaconic acid derivatives have been demonstrated to elicit anti-inflammatory effects (Bagavant G. et al., 1994). A notable example is 4-octyl itaconic acid (4OI), an itaconate derivative with improved cellular uptake. Since the α,β-unsaturated carboxylic acid is not esterified in 4OI, this electrophile exhibits low reactivity with biological thiols (Schmidt T. J. et al., 2007), much like the situation encountered with itaconic acid itself. As a result of its low reactivity/electrophilicity, the NRF2-activating effects of 4OI are not attenuated by GSH, in contrast to the findings with the much more reactive DMI. In this latter case, the α,β-unsaturated carboxylic acid is esterified and, as a consequence, the IL-6-lowering and NRF2-activating effects of DMI are reversed by the thiols N-acetylcysteine and GSH, respectively. Through the reaction with KEAP1 and the resulting NRF2 activation, as well as GAPDH inhibition (Liao S.-T. et al., 2019), 4OI has been demonstrated to produce a wide range of interesting biological effects, including: 1) protection of neuronal cells from hydrogen peroxide (Liu H. et al., 2018); 2) inhibition of proinflammatory cytokine production in peripheral blood mononuclear cells of SLE patients (Tang C. et al., 2018); and 3) protection of human umbilical vein endothelial cells from high glucose (Tang C. et al., 2019); 4) inhibition of osteoclastogenesis by suppressing the E3 ubiquitin ligase Hrd1 and activating NRF2 signaling (Sun X. et al., 2019); 5) induction of repression of STING by NRF2 and type I IFN production in cells from patients with STING-dependent interferonopathies (Olagnier D. et al., 2018); 6) protection against renal fibrosis via inhibiting the TGF-beta/Smad pathway, autophagy and reducing generation of reactive oxygen species (Tian F. et al., 2020); 7) reduction of brain viral burden in mice intracranially injected with Zika virus (Daniels B. P. et al. 2019); and 8) protection against liver ischemia-reperfusion injury (Yi F. et al. 2020). Furthermore, itaconate has been reported to modulate tricarboxylic acid and redox metabolism to mitigate reperfusion injury (Cordes T. et al., 2020). In addition, raised plasma itaconate levels demonstrate a clear correlation with reduction in rheumatoid arthritis disease activity scores following commencement of therapy with conventional disease modifying anti-rheumatic drug (cDMARD) therapy (Daly R. et al. 2019).

Artyomov et al. (WO2017/142855; WO2019/036509) disclose the use of itaconate, malonate or a derivative thereof as an immunomodulatory agent.

In spite of the above findings, there remains a need to identify and develop new itaconate derivatives possessing enhanced properties compared to currently marketed anti-inflammatory agents, such as dimethyl itaconate. The present inventors have now discovered, surprisingly, that certain itaconate diesters with good metabolic stability are highly effective at reducing cytokine release in cells and/or in activating NRF2-driven effects. These properties, amongst others, make them potentially more effective than dimethyl itaconate. Such compounds therefore possess excellent anti-inflammatory properties.

### SUMMARY OF THE INVENTION

The present invention provides a compound of formula (I):

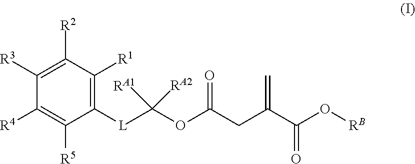

wherein:
R^{A1} and R^{A2} join to form a C₃₋₅ cycloalkyl ring:

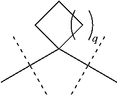

wherein q is 0, 1 or 2; or
R^{A1} and R^{A2} join to form a 4-6-membered heterocyclic ring;
   wherein the C₃₋₅ cycloalkyl ring and 4-6-membered heterocyclic ring are optionally substituted by one or more groups selected from methyl and fluoro;
R¹ is H or Cl;
R² is H or halo;
R³ is H, halo, CH₃, OCH₃, CF₃, OCF₃, SC₁₋₄alkyl, SC₁₋₄haloalkyl, phenyl which is optionally substituted by halo, or —C≡C—C₁₋₂haloalkyl;
R⁴ is H, halo or CF₃;
R⁵ is H or Cl;
   wherein one, two or three of any of the R¹ to R⁵ groups are other than H and the remainder are H;
L is a bond, C₁₋₂alkylene or C≡C; and
R^{B} is selected from the group consisting of CH₂COOH, CH₂CH₂COOH, CH₂tetrazolyl and CH₂CH₂tetrazolyl wherein R^{B} is optionally substituted on an available carbon atom by one or more R^{B1} wherein R^{B1} is selected from the group consisting of difluoromethyl, trifluoromethyl and methyl, and/or two R^{B1} groups which are attached to the same carbon atom join to form a C₃₋₆ cycloalkyl ring or a 4-6-membered heterocyclic ring;
   or a pharmaceutically acceptable salt and/or solvate thereof.

The present invention provides a pharmaceutical composition comprising a compound of formula (I) or a pharmaceutically acceptable salt and/or solvate thereof.

The present invention provides a compound of formula (I) or a pharmaceutically acceptable salt and/or solvate thereof for use as a medicament.

The present invention provides a compound of formula (I) or a pharmaceutically acceptable salt and/or solvate thereof for use in treating or preventing an inflammatory disease or a disease associated with an undesirable immune response.

The present invention provides the use of a compound of formula (I) or a pharmaceutically acceptable salt and/or solvate thereof in the manufacture of a medicament for treating or preventing an inflammatory disease or a disease associated with an undesirable immune response.

The present invention provides a method of treating or preventing an inflammatory disease or a disease associated with an undesirable immune response, which comprises administering a compound of formula (I) or a pharmaceutically acceptable salt and/or solvate thereof.

Also provided are intermediate compounds of use in the preparation of compounds of formula (I).

### DETAILED DESCRIPTION OF THE INVENTION

Compounds of Formula (I)

Embodiments and preferences set out herein with respect to the compound of formula (I) apply equally to the pharmaceutical composition, compound for use, use and method aspects of the invention.

In one embodiment, there is provided a compound of formula (I) as defined above.

Suitably, R³ is SC₁₋₄alkyl, SC₁₋₄haloalkyl, phenyl which is optionally substituted by halo, or —C≡C—C₁₋₂haloalkyl; L is C₁₋₂alkylene or C≡C; and/or two R^{B1} groups which are attached to the same carbon atom join to form a C₃₋₆ cycloalkyl ring or a 4-6-membered heterocyclic ring. Alternatively, R³ is other than SC₁₋₄alkyl, SC₁₋₄haloalkyl, phenyl which is optionally substituted by halo, or —C≡C—C₁₋₂haloalkyl; L is other than C₁₋₂alkylene or C≡C; and/or R^{B} is substituted by groups other than two R^{B1} groups which are attached to the same carbon atom join to form a C₃₋₆ cycloalkyl ring or a 4-6-membered heterocyclic ring.

Suitably, R³ is selected from H, CH₃, OCH₃, CF₃, OCF₃ and halo; L is a bond; and R^{B1} is selected from the group consisting of difluoromethyl, trifluoromethyl and methyl.

In one embodiment, there is provided a compound of formula (Ia):

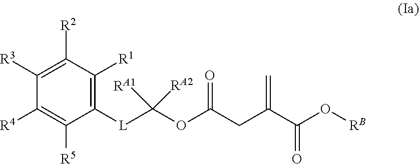

wherein:
R^{A1} and R^{A2} join to form a C₃₋₅ cycloalkyl ring:

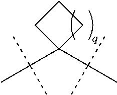

wherein q is 0, 1 or 2; or
R^{A1} and R^{A2} join to form a 4-6-membered heterocyclic ring;
   wherein the C₃₋₅ cycloalkyl ring and 4-6-membered heterocyclic ring are optionally substituted by one or more groups selected from methyl and fluoro;
R¹ is H or Cl;
R² is H or halo;
R³ is H, halo, CH₃, OCH₃, CF₃, OCF₃, phenyl which is optionally substituted by halo, or —C≡C—C₁₋₂haloalkyl;
R⁴ is H, halo or CF₃;
R⁵ is H or Cl;
   wherein one, two or three of any of the R¹ to R⁵ groups are other than H and the remainder are H;
L is a bond, C₁₋₂alkylene or C≡C; and
R^{B} is CH₂COOH or CH₂CH₂COOH, wherein R^{B} is optionally substituted on an available carbon atom by one or more R^{B1} wherein R^{B1} is selected from the group consisting of difluoromethyl, trifluoromethyl and methyl, and/or two R^{B1} groups which are attached to the same carbon atom join to form a C₃₋₆ cycloalkyl ring or a 4-6-membered heterocyclic ring;
   or a pharmaceutically acceptable salt and/or solvate thereof.

Suitably, R³ is phenyl which is optionally substituted by halo, or —C≡C—C₁₋₂haloalkyl; L is C₁₋₂alkylene or C≡C; and/or two R^{B1} groups which are attached to the same carbon atom join to form a C₃₋₆ cycloalkyl ring or a 4-6-membered heterocyclic ring. Alternatively, R³ is other than phenyl which is optionally substituted by halo, or —C≡C—C₁₋₂haloalkyl; L is other than C₁₋₂alkylene or C≡C; and/or R^{B} is substituted by groups other than two R^{B1} groups which are attached to the same carbon atom join to form a C₃₋₆ cycloalkyl ring or a 4-6-membered heterocyclic ring.

Suitably, R³ is selected from H, CH₃, OCH₃, CF₃, OCF₃ and halo; L is a bond; and R^{B1} is selected from the group consisting of difluoromethyl, trifluoromethyl and methyl.

In one embodiment, there is provided a compound of formula (Ib):

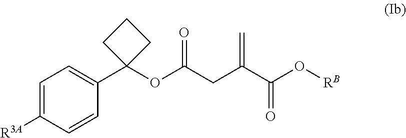

wherein:
R^{3A} is SF₅, SC₁₋₄alkyl or SC₁₋₄haloalkyl; and
R^{B} is selected from the group consisting of CH₂COOH, CH₂CH₂COOH, CH₂tetrazolyl and CH₂CH₂tetrazolyl, wherein R^{B} is optionally substituted on an available carbon atom by one or more R^{B′} wherein R^{B′} is selected from the group consisting of difluoromethyl, trifluoromethyl and methyl; and/or wherein R^{B} is optionally substituted by two R^{B′} groups, attached to the same carbon atom, that are joined to form a C₃₋₆ cycloalkyl or a 4-6-membered heterocyclyl ring;
   or a pharmaceutically acceptable salt and/or solvate thereof.

In one embodiment, there is provided a compound of formula (Ic):

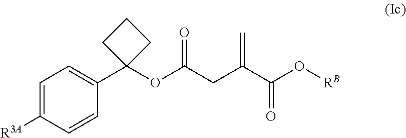

wherein:
R^{3A} is SC₁₋₄alkyl or SC₁₋₄haloalkyl; and
R^{B} is selected from the group consisting of CH₂COOH, CH₂CH₂COOH, CH₂tetrazolyl and CH₂CH₂tetrazolyl, wherein R^{B} is optionally substituted on an available carbon atom by one or more R^{B′} wherein R^{B′} is selected from the group consisting of difluoromethyl, trifluoromethyl and methyl; and/or wherein R^{B} is optionally substituted by two R^{B′} groups, attached to the same carbon atom, that are joined to form a C₃₋₆ cycloalkyl or a 4-6-membered heterocyclyl ring;
   or a pharmaceutically acceptable salt and/or solvate thereof.

In one embodiment, there is provided a compound of formula (Id):

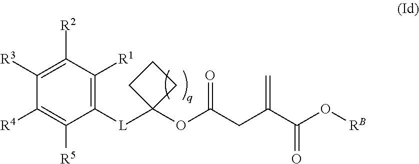

wherein:
R¹ is H or Cl;
R² is H or halo;
R³ is H, halo, CH₃, OCH₃, CF₃, OCF₃, phenyl which is optionally substituted by halo, or —C≡C—C₁₋₂haloalkyl;
R⁴ is H, halo or CF₃;
R⁵ is H or Cl;
   wherein one, two or three of any of the R¹ to R⁵ groups are other than H and the remainder are H;
L is a bond, C₁₋₂alkylene or C≡C;
q is 0, 1 or 2; and
R^{B} is CH₂COOH or CH₂CH₂COOH, wherein R^{B} is optionally substituted on an available carbon atom by one or more R^{B1} wherein R^{B1} is selected from the group consisting of difluoromethyl, trifluoromethyl and methyl;
   or a pharmaceutically acceptable salt and/or solvate thereof.

Suitably, R³ is phenyl which is optionally substituted by halo, or —C≡C—C₁₋₂haloalkyl; and/or L is C₁₋₂alkylene or C≡C. Alternatively, R³ is other than phenyl which is optionally substituted by halo, or —C≡C—C₁₋₂haloalkyl; and/or L is other than C₁₋₂alkylene or C≡C.

Suitably, R³ is H, CH₃, OCH₃, CF₃, OCF₃ and halo; L is bond; and R^{B1} is selected from the group consisting of difluoromethyl, trifluoromethyl and methyl.

In one embodiment is provided a compound formula (Ie):

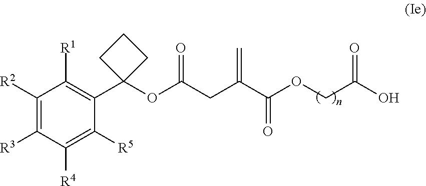

wherein:
R¹ is H or Cl;
R² is H or halo;
R³ is H, halo, CH₃, CF₃ or OCF₃;
R⁴ is H, halo or CF₃;
R⁵ is H or Cl;
   wherein one, two or three of any of the R¹ to R⁵ groups are other than H and the remainder are H; and
n is 1 or 2;
   or a pharmaceutically acceptable salt and/or solvate thereof.

References and fall-back positions relating to compounds of formula (I) apply equally to compounds of formula (Ia) to (If).

The term “C₁₋₄ alkyl” (e.g. C₁₋₃ alkyl group, C₁₋₂ alkyl group or C, alkyl group) as used herein refers to a straight or a branched fully saturated hydrocarbon chain containing the specified number of carbon atoms. Examples of C₁0.4 alkyl include methyl, ethyl, n-propyl, i-propyl, n-butyl, t-butyl and s-butyl. The term “C₁₋₂alkylene” as used herein means —CH₂—(C₁alkylene), —CH₂CH₂—(C₂alkylene) or —CH(Me)—(C₂alkylene).

The term “halo” as used herein means a halogen atom such as fluoro (F), chloro (Cl), bromo (Br) or iodo (I). Suitably, the halogen atom is fluoro (F), chloro (Cl) or bromo (Br).

The term “haloalkyl”, such as “C₁₋₄ haloalkyl” or “C₁₋₂ haloalkyl”, as used herein refers to a straight or a branched fully saturated hydrocarbon chain containing the specified number of carbon atoms and at least one halogen atom, such as fluoro or chloro, especially fluoro. An example of haloalkyl is CF₃.

The term “cycloalkyl”, such as “C₃₋₆ cycloalkyl” or “C₃₋₅ cycloalkyl” refers to a fully saturated cyclic hydrocarbon group having the specified number of carbon atoms. The term encompasses cyclopropyl, cyclobutyl, cyclopentyl and cyclohexyl.

The term “4-6-membered heterocyclic ring” refers to a non-aromatic cyclic group having 4 to 6 ring atoms and wherein at least one of the ring atoms is a heteroatom selected from N, O, S and B. The term “heterocyclic ring” is interchangeable with “heterocyclyl”. The term encompasses azetidinyl, oxetanyl, thietanyl, pyrrolidinyl, tetrahydrofuranyl, tetrahydrothienyl, tetrahydropyranyl, piperidinyl, piperazinyl, morpholinyl and thiomorpholinyl. 4-6-membered heterocyclyl groups can typically be substituted by one or more (e.g. one or two) oxo groups. Suitably, thietanyl is substituted by one or two oxo groups such as a sulfur atom is substituted by one or two oxo groups thus forming S═O or SO₂.

The term “tetrazolyl” refers to a 5-(1H-tetrazolyl) substituent where the tetrazole is linked to the rest of the molecule via a carbon atom:

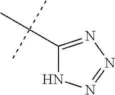

wherein the dashed line indicates the point of attachment to the rest of the molecule.

In one embodiment, R^{A1} and R^{A2} join to form a C₃₋₅ cycloalkyl ring:

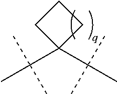

wherein q is 0, 1 or 2.

In one embodiment, R^{A1} and R^{A2} join to form a C₃ cycloalkyl ring (q is 0). In a second embodiment, R^{A1} and R^{A2} join to form a C₄ cycloalkyl ring (q is 1). In a third embodiment, R^{A1} and R^{A2} join to form a C₅ cycloalkyl ring (q is 2). Suitably, R^{A1} and R^{A2} join to form a C₄ cycloalkyl ring (q is 1).

In one embodiment, the C₃₋₅ cycloalkyl ring is not substituted. In another embodiment, the C₃₋₅ cycloalkyl ring is substituted by one or more (such as one or two, e.g., one) groups selected from methyl and fluoro. In one embodiment, the C₃₋₅ cycloalkyl ring is substituted by one or more (such as one or two, e.g., one) methyl groups. In another embodiment, the C₃₋₅ cycloalkyl ring is substituted by one or more (such as one or two, e.g., two) fluoro groups. In a third embodiment, the C₃₋₅ cycloalkyl ring is substituted by one methyl group and one fluoro group.

Suitably, the C₃₋₅ cycloalkyl ring is not substituted.

In another embodiment, R^{A1} and R^{A2} join to form a 4-6-membered heterocyclic ring. In one embodiment, R^{A1} and R^{A2} join to form a 4-membered heterocyclic ring, such as oxetanyl. In a second embodiment, R^{A1} and R^{A2} join to form a 5-membered heterocyclic ring. In a third embodiment, R^{A1} and R^{A2} join to form a 6-membered heterocyclic ring. Suitably, R^{A1} and R^{A2} join to form a 4-membered heterocyclic ring, such as oxetanyl.

In one embodiment, the 4-6-membered heterocyclic ring is not substituted. In another embodiment, the 4-6-membered heterocyclic ring is substituted by one or more (such as one or two, e.g., one) groups selected from methyl and fluoro. In one embodiment, the 4-6-membered heterocyclic ring is substituted by one or more (such as one or two, e.g., one) methyl groups. In another embodiment, the 4-6-membered heterocyclic ring is substituted by one or more (such as one or two, e.g., two) fluoro groups. In a third embodiment, the 4-6-membered heterocyclic ring is substituted by one methyl group and one fluoro group.

Suitably, the 4-6-membered heterocyclic ring is not substituted.

In one embodiment, R¹ is H. In another embodiment, R¹ is Cl.

In one embodiment, R² is H. In another embodiment, R² is halo, such as fluoro or chloro, suitably chloro.

In one embodiment, R³ is H. In a second embodiment, R³ is halo, such as fluoro, chloro or bromo, for example chloro or bromo, suitably chloro. In a third embodiment, R³ is CH₃. In a fourth embodiment, R³ is OCH₃. In a fifth embodiment, R³ is CF₃. In a sixth embodiment, R³ is OCF₃. In a seventh embodiment, R³ is SC₁₋₄alkyl such as SCH₃. In an eighth embodiment, R³ is SC₁₋₄haloalkyl such as SCF₃. In a ninth embodiment, R³ is phenyl which is optionally substituted by halo. Suitably, the phenyl ring is substituted by halo e.g. fluoro. Most suitably, the halo atom is in the para position with respect to the phenyl ring to which R³ is attached. In a tenth embodiment, R³ is —C≡C—C₁₋₂haloalkyl, e.g., —C≡C—CF₃.

In another embodiment, R³ is R^{3A} and is SC₁₋₄alkyl such as SCH₃. In another embodiment, R³ is R^{3A} and is SC₁₋₄haloalkyl such as SCF₃.

In one embodiment, R⁴ is H. In another embodiment, R⁴ is halo, such as fluoro or chloro, suitably chloro. In a third embodiment, R⁴ is CF₃.

In one embodiment, R⁵ is H. In another embodiment, R⁵ is Cl.

In any one of the above embodiments, one, two or three of any of the R¹ to R⁵ groups are other than H and the remainder are H. Suitably, one of any of the R¹ to R⁵ groups is other than H and the remainder are H. Alternatively, two of any of the R¹ to R⁵ groups are other than H and the remainder are H. Alternatively, three of any of the R¹ to R⁵ groups are other than H and the remainder are H.

In one embodiment, L is a bond. In a second embodiment, L is C₁₋₂alkylene such as CH₂. In a third embodiment, L is C≡C.

In one embodiment, q is 0. In a second embodiment, q is 1. In a third embodiment, q is 2.

In one embodiment, R^{B} is CH₂COOH (i.e. n is 1). In a second embodiment, R^{B} is CH₂CH₂COOH (i.e. n is 2). In a third embodiment, R^{B} is CH₂tetrazolyl. In a fourth embodiment, R^{B} is CH₂CH₂tetrazolyl.

In one embodiment, R^{B} is not substituted. In a second embodiment, R^{B} is substituted on an available carbon atom by one or more (such as one, two, three or four, e.g. one or two, e.g., one) R^{B1} wherein R^{B1} is selected from the group consisting of difluoromethyl, trifluoromethyl and methyl. In one embodiment, R^{B1} is difluoromethyl. In a second embodiment, R^{B1} is trifluoromethyl. In a third embodiment, R^{B1} is methyl. In a fourth embodiment, two R^{B1} groups which are attached to the same carbon atom join to form a C₃₋₆ cycloalkyl ring or a 4-6-membered heterocyclic ring. In one embodiment, two R^{B1} groups which are attached to the same carbon atom join to form a C₃₋₆ cycloalkyl ring (e.g. a cyclopropyl ring). In another embodiment, two R^{B1} groups which are attached to the same carbon atom join to form a 4-6-membered heterocyclic ring, such as a 4-membered heterocyclic ring e.g., oxetanyl. Suitably, R^{B1} is trifluoromethyl.

In one embodiment, R^{B1} is R^{B′}. In one embodiment, R^{B} is not substituted. In a second embodiment, R^{B} is substituted on an available carbon atom by one or more (such as one, two, three or four, e.g. one or two, e.g., one) R^{B′} wherein R^{B′} is selected from the group consisting of difluoromethyl, trifluoromethyl and methyl. In one embodiment, R^{B′} is difluoromethyl. In a second embodiment, R^{B′} is trifluoromethyl. In a third embodiment, R^{B′} is methyl. In a fourth embodiment, two R^{B1} groups which are attached to the same carbon atom join to form a C₃₋₆ cycloalkyl ring or a 4-6-membered heterocyclic ring. In one embodiment, two R^{B′} groups which are attached to the same carbon atom join to form a C₃₋₆ cycloalkyl ring. In another embodiment, two R^{B′} groups which are attached to the same carbon atom join to form a 4-6-membered heterocyclic ring, such as a 4-membered heterocyclic ring e.g., oxetanyl. Suitably, R^{B′} is trifluoromethyl.

As used herein, the term “available carbon” means any C—H bond in R^{B} wherein the H atom may be replaced by R^{B1}. In particular, a C—H bond of CH₂ or CH₂CH₂ within the R^{B} moiety is replaced by C—R^{B1}.

Suitably, the R^{B} group is to the carbon atom of R^{B} adjacent to the ester oxygen atom, i.e., such that the following moiety forms:

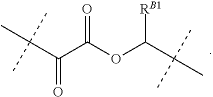

In one embodiment, R^{B} is CH₂COOH (i.e., n is 1), R₃ is CF₃ and R¹, R², R⁴ and R⁵ are H.

In another embodiment, R^{B} is CH(CF₃)CH₂COOH (i.e. n is 2), R³ is CF₃ and R¹, R², R⁴ and R⁵ are H.

Most suitably, R^{B} is CH₂COOH (i.e. n is 1), R² and R⁴ are chloro and R¹, R³ and R⁵ are H.

Alternatively, R^{B} is CH₂CH₂COOH (i.e. n is 2), R³ is CF₃ and R¹, R², R⁴ and R⁵ are H.

In one embodiment there is provided a compound of formula (If):

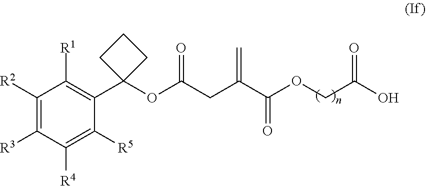

wherein:
R¹ is H or Cl;
R² is H or Cl;
R³ is H, Cl, Br, CF₃ or OCF₃;
R⁴ is H or Cl;
R⁵ is H or Cl;
   wherein one or two of any of the R¹ to R⁵ groups are other than H and the remainder are H; and
n is 1 or 2;
   or a pharmaceutically acceptable salt and/or solvate thereof.

In one embodiment there is provided a compound of formula (I), selected from the group consisting of:
2-((4-(1-(3,5-dichlorophenyl)cyclobutoxy)-2-methylene-4-oxobutanoyl)oxy)acetic acid;
2-((2-methylene-4-oxo-4-(1-(4-(trifluoromethyl)phenyl)cyclobutoxy) butanoyl)oxy)acetic acid;
2-((4-(1-(4-bromophenyl)cyclobutoxy)-2-methylene-4-oxobutanoyl)oxy)acetic acid;
2-((4-(1-(4-chlorophenyl)cyclobutoxy)-2-methylene-4-oxobutanoyl)oxy)acetic acid;
2-((4-(1-(2,5-dichlorophenyl)cyclobutoxy)-2-methylene-4-oxobutanoyl)oxy)acetic acid;
2-((4-(1-(2-chloro-4-(trifluoromethoxy)phenyl)cyclobutoxy)-2-methylene-4-oxobutanoyl)oxy)acetic acid;
2-((4-(1-(2,3-dichlorophenyl)cyclobutoxy)-2-methylene-4-oxobutanoyl)oxy)acetic acid;
2-((4-(1-(2,6-dichlorophenyl)cyclobutoxy)-2-methylene-4-oxobutanoyl)oxy)acetic acid;
2-((2-methylene-4-oxo-4-(1-(4-(trifluoromethoxy)phenyl)cyclobutoxy) butanoyl)oxy)acetic acid;
2-((4-(1-(2,4-dichlorophenyl)cyclobutoxy)-2-methylene-4-oxobutanoyl)oxy)acetic acid;
2-((4-(1-(2-chloro-4-(trifluoromethyl)phenyl)cyclobutoxy)-2-methylene-4-oxobutanoyl)oxy)acetic acid;
2-((4-(1-(4-bromo-2-chlorophenyl)cyclobutoxy)-2-methylene-4-oxobutanoyl)oxy)acetic acid;
3-((4-(1-(3,5-dichlorophenyl)cyclobutoxy)-2-methylene-4-oxobutanoyl)oxy)propanoic acid;
2-((2-methylene-4-oxo-4-(1-(3,4,5-trifluorophenyl)cyclobutoxy)butanoyl)oxy) acetic acid; and
2-((4-(1-(3-chloro-5-(trifluoromethyl)phenyl)cyclobutoxy)-2-methylene-4-oxobutanoyl)oxy)acetic acid;
   or a pharmaceutically acceptable salt and/or solvate thereof.

In another embodiment there is provided a compound of formula (I), selected from the group consisting of:
2-((4-(1-(3-chloro-4-(trifluoromethyl)phenyl)cyclobutoxy)-2-methylene-4-oxobutanoyl)oxy)acetic acid;
2-((4-(1-(3,5-dichloro-4-fluorophenyl)cyclobutoxy)-2-methylene-4-oxobutanoyl)oxy)acetic acid;
2-((4-(1-(3,5-dichloro-4-methoxyphenyl)cyclobutoxy)-2-methylene-4-oxobutanoyl)oxy)acetic acid;
2-((4-(1-(3,5-difluoro-4-(trifluoromethyl)phenyl)cyclobutoxy)-2-methylene-4-oxobutanoyl)oxy)acetic acid;
2-((4-(1-(3-chloro-4-methylphenyl)cyclobutoxy)-2-methylene-4-oxobutanoyl)oxy)acetic acid;
(S)-4,4,4-trifluoro-3-((2-methylene-4-oxo-4-(1-(4-(trifluoromethyl)phenyl)cyclobutoxy)butanoyl)oxy)butanoic acid;
3-((2-methylene-4-oxo-4-(1-(4-(trifluoromethyl)phenyl)cyclobutoxy)butanoyl)oxy)propanoic acid;
(R)-4,4,4-trifluoro-3-((2-methylene-4-oxo-4-(1-(4-(trifluoromethyl)phenyl)cyclobutoxy)butanoyl)oxy)butanoic acid;
2-((4-(1-(4-chlorobenzyl)cyclobutoxy)-2-methylene-4-oxobutanoyl)oxy)acetic acid;
2-((2-methylene-4-oxo-4-((1-(4-(trifluoromethyl)phenyl)cyclopentyl)oxy)butanoyl)oxy)acetic acid;
2-((4-(1-(4′-fluoro-[1,1′-biphenyl]-4-yl)cyclobutoxy)-2-methylene-4-oxobutanoyl)oxy)acetic acid;
2-((2-methylene-4-oxo-4-(1-(4-(trifluoromethyl)benzyl)cyclopropoxy)butanoyl)oxy)acetic acid; and
2-((2-methylene-4-oxo-4-(1-((4-(trifluoromethyl)phenyl)ethynyl)cyclobutoxy)butanoyl)oxy)acetic acid;
   or a pharmaceutically acceptable salt and/or solvate thereof.

In another embodiment there is provided a compound of formula (I), selected from the group consisting of:
2-((2-methylene-4-oxo-4-((3-(4-(trifluoromethyl)phenyl)oxetan-3-yl)oxy)butanoyl)oxy)acetic acid;
2-((2-methylene-4-oxo-4-(1-(4-(trifluoromethyl)phenyl) cyclopropoxy)butanoyl)oxy)acetic acid;
2-((4-(3,3-difluoro-1-(4-(trifluoromethyl)phenyl)cyclobutoxy)-2-methylene-4-oxobutanoyl)oxy)acetic acid;
3-((2-methylene-4-oxo-4-((3-(4-(trifluoromethyl)phenyl)oxetan-3-yl)oxy)butanoyl)oxy)propanoic acid; and
2-(3-((2-methylene-4-oxo-4-(1-(4-(trifluoromethyl)phenyl)cyclobutoxy) butanoyl)oxy)oxetan-3-yl)acetic acid;
   or a pharmaceutically acceptable salt and/or solvate thereof.

In one embodiment, the compound is selected from the group consisting of:
2-((2-methylene-4-oxo-4-(1-(4-((trifluoromethyl)thio)phenyl)cyclobutoxy)butanoyl)oxy)acetic acid;
2-((2-methylene-4-oxo-4-(1-(4-(pentafluoro-λ⁶-sulfaneyl)phenyl)cyclobutoxy)butanoyl)oxy)acetic acid;
3-((2-methylene-4-oxo-4-(1-(4-((trifluoromethyl)thio)phenyl)cyclobutoxy)butanoyl)oxy)propanoic acid; and
3-((2-methylene-4-oxo-4-(1-(4-(pentafluoro-λ⁶-sulfaneyl)phenyl)cyclobutoxy)butanoyl)oxy)propanoic acid;
   or a pharmaceutically acceptable salt and/or solvate of any one thereof.

In one embodiment, the compound is selected from the group consisting of:
2-((2-methylene-4-oxo-4-(1-(4-((trifluoromethyl)thio)phenyl)cyclobutoxy)butanoyl)oxy)acetic acid; and
3-((2-methylene-4-oxo-4-(1-(4-((trifluoromethyl)thio)phenyl)cyclobutoxy)butanoyl)oxy)propanoic acid;
   or a pharmaceutically acceptable salt and/or solvate of any one thereof.

Suitably, the compound is 2-((2-methylene-4-oxo-4-(1-(4-((trifluoromethyl)thio)phenyl) cyclobutoxy)butanoyl)oxy)acetic acid.

Compounds of formula (I) may be made as set out in the examples and also as set out in the Schemes below.

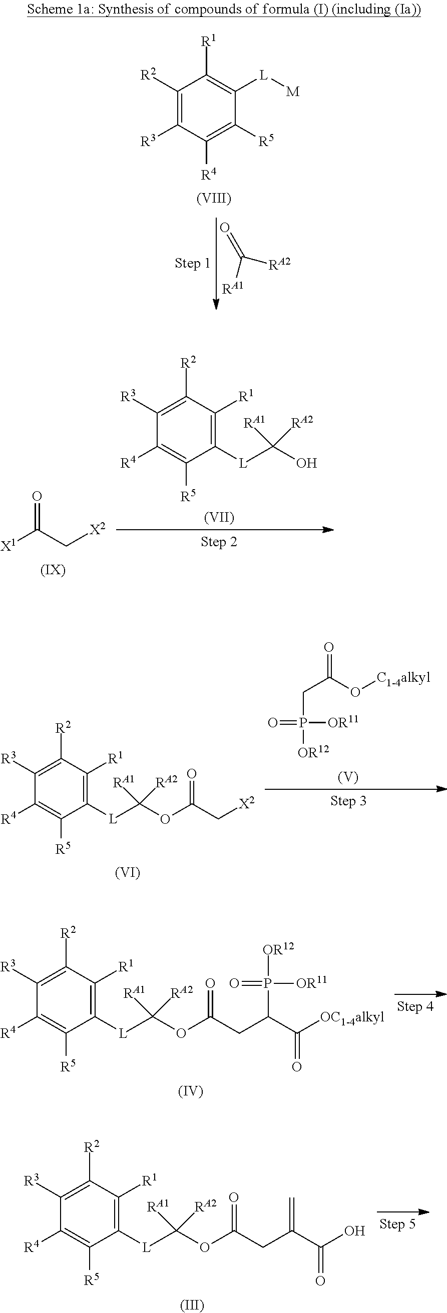

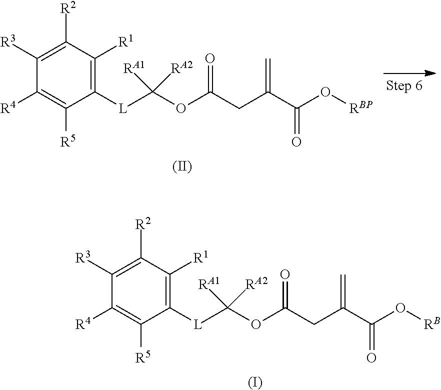

R¹ to R⁵, L, R^{A1}, R^{A2} and R^{B} are as defined elsewhere herein. R^{BP} is a protected derivative of R^{B} such as the carboxylic acid group of R^{B} is protected with a carboxylic acid protecting group. Suitable carboxylic acid protecting groups are listed herein, and suitably the carboxylic acid protecting group is CH₂CCl₃. Alternatively, the carboxylic acid protecting group is Fmoc. M, X¹, X², R¹¹ and R¹² are as defined below.

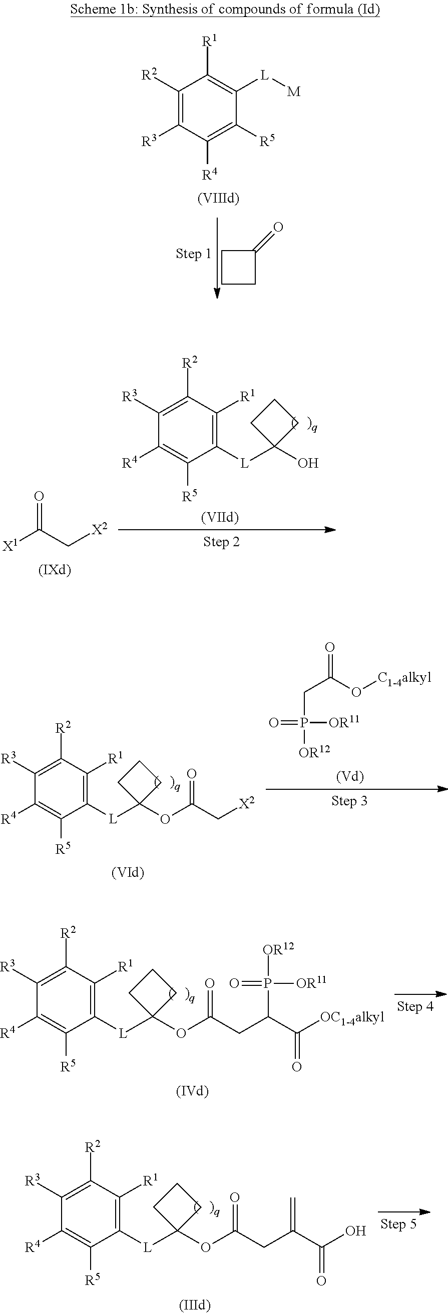

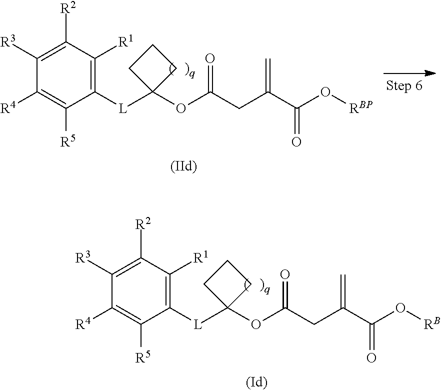

R¹ to R⁵, L, q and R^{B} are as defined elsewhere herein. R^{BP} is a protected derivative of R^{B} such as the carboxylic acid group of R^{B} is protected with a carboxylic acid protecting group. Suitable carboxylic acid protecting groups are listed herein, and suitably the carboxylic acid protecting group is CH₂CCl₃. Alternatively, the carboxylic acid protecting group is Fmoc. M, X¹, X², R¹¹ and R¹² are as defined below.

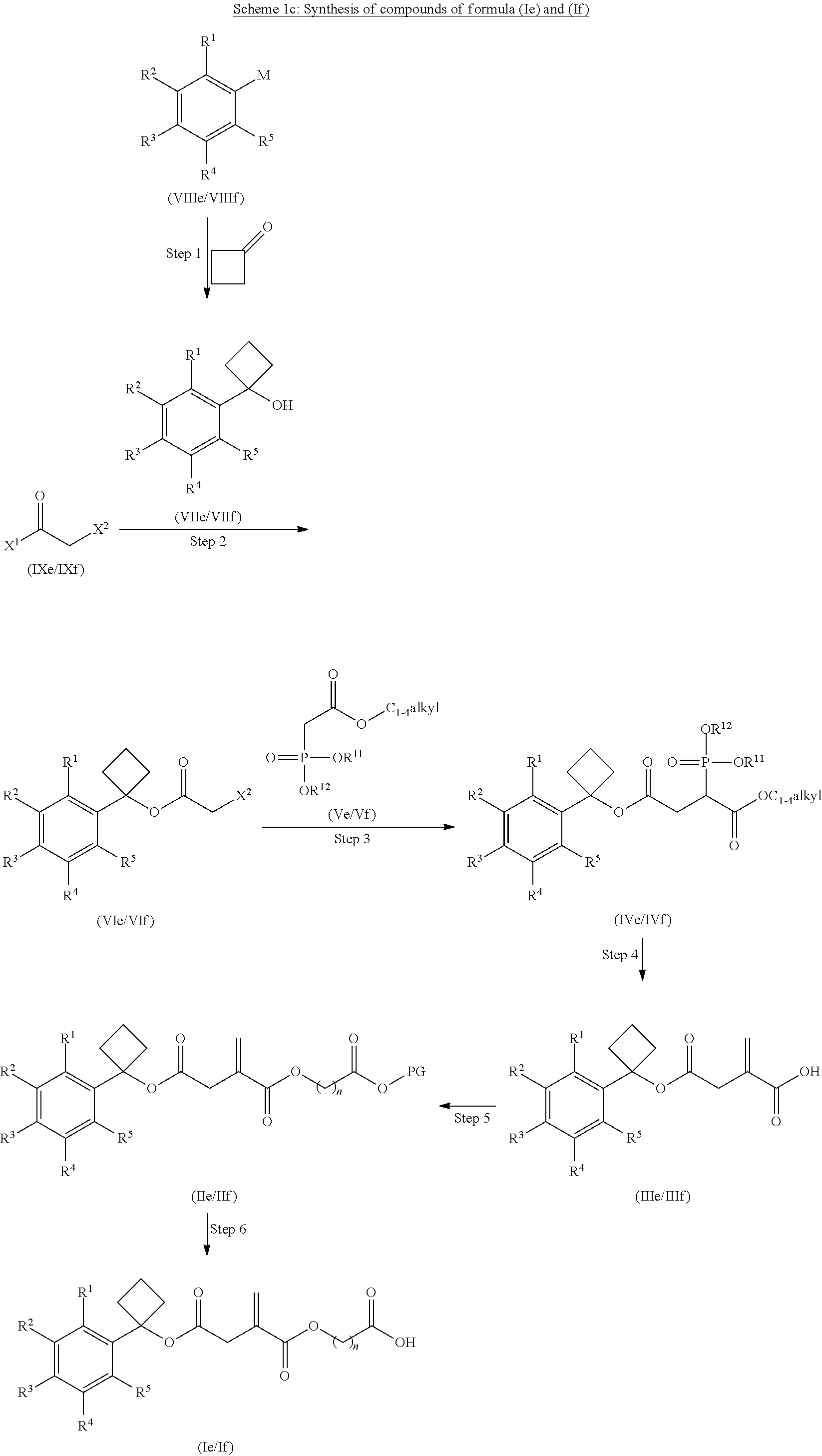

R¹ to R⁵ and n are as defined elsewhere herein. M, X¹, X², R¹¹, R¹² and PG are as defined below.

The following description applies to both Schemes 1a, 1b and 1c, unless otherwise stated. The numbering used below applies to compounds in Schemes 1a, 1b and 1c. For example, reference to compounds of formula (VII) below encompasses compounds of formulae (VII), (VIIa), (VIId), (VIIe) and (VIIf).

Step 1: Commercially available Grignard reagent (VIII; M=MgBr) or organolithium (VIII; M=Li)—prepared from the corresponding aryl bromide or iodide—is reacted with cyclobutanone to give compounds of formula (VII).

Step 2: Alcohol (VII) is condensed with compound (IX), wherein X¹ and X² represent leaving groups, such as halo, e.g., chloro, bromo or iodo, to give monoester (VI).

Step 3: Monoester (VI) is reacted with a trialkylphosphonoacetate of formula (V), wherein R¹¹ and R¹² independently represent C₁₋₄ alkyl optionally substituted with halo, to provide a compound of formula (IV).

Step 4: Condensation of a compound of formula (IV) with formaldehyde or a formaldehyde equivalent e.g., paraformaldehyde, followed by hydrolysis of the C₁₋₄ alkyl ester and any optional deprotection steps, provides the compound of formula (III).

Step 5:

Scheme 1a and 1b: R^{BP} is introduced by reaction of compounds of formula (III) with R^{BP}—X³ wherein R^{BP} is a protected derivative of R^{B}, and X³ is OH or a leaving group such as OMs, OTs or halo, such as bromo, to give compounds of formula (II).

Scheme 1c: The acetate or propionate carboxylic acid moiety is introduced by reaction of compounds of formula (III) with a suitably protected acetic or propionic acid moiety for example 2,2,2-trichloroethyl 2-bromoacetate or 2,2,2-trichloroethyl 3-hydroxypropanoate, wherein PG is a carboxylic acid protecting group for example CH₂CCl₃, to give compounds of formula (II). Other carboxylic acid protecting groups may be used and are known to the person skilled in the art, and are described elsewhere herein.

Step 6:

Scheme 1a: Compounds of formula (II) are converted to compounds of formula (I) by deprotection e.g. removal of the carboxylic acid protecting group for example using Zn-mediated reduction or trimethyltin hydroxide in 1,2-dichloroethane when R^{B} is protected with CH₂CCl₃.

Scheme 1b: Compounds of formula (II) are converted to compounds of formula (I) by removal of PG, for example using Zn-mediated reduction or trimethyltin hydroxide in 1,2-dichloroethane when PG is CH₂CCl₃.

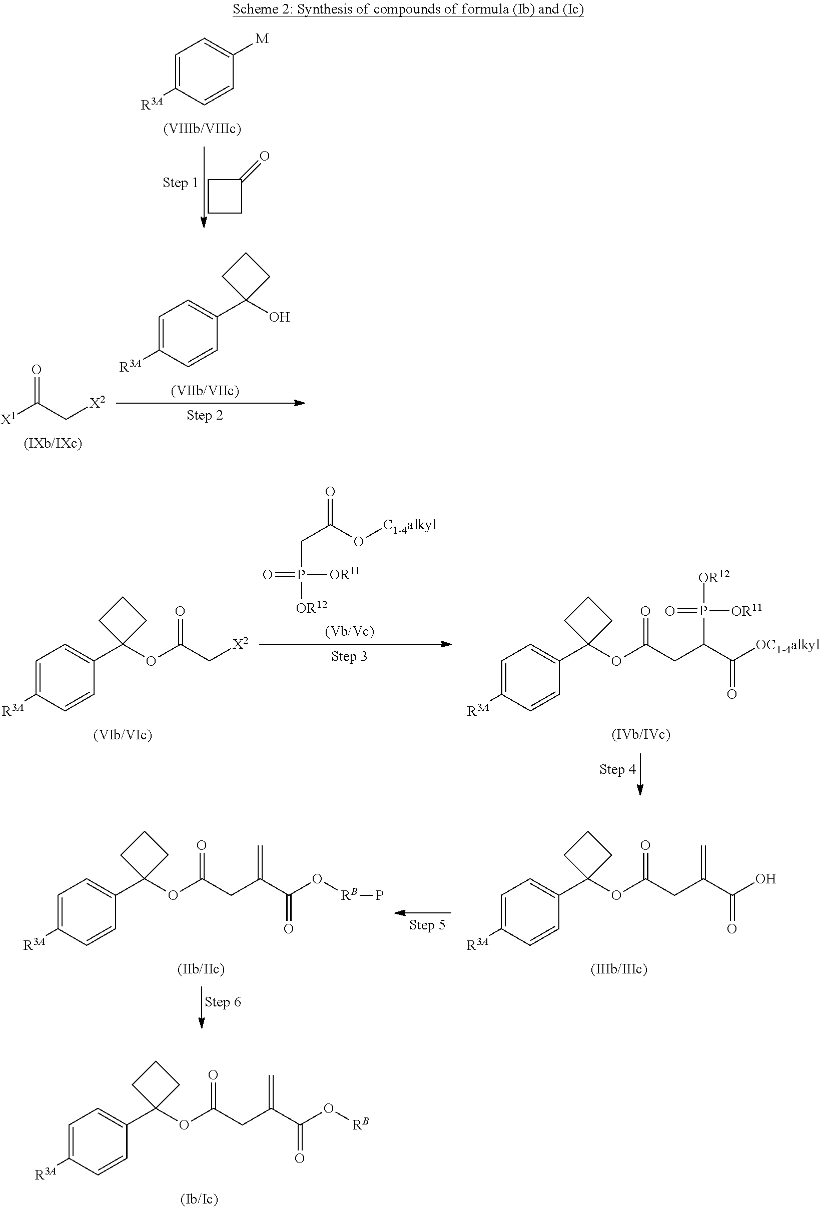

R^{3A} and R^{B} are as defined elsewhere herein, and P represents a carboxylic acid protecting group such as C₁₋₆ alkyl e.g. tert-butyl, C₁₋₆ haloalkyl, such as CH₂CCl₃, or para-methoxybenzyl, or a tetrazolyl protecting group such as para-methoxybenzyl or trityl. Suitably, P represents a carboxylic acid protecting group such as C₁₋₆ alkyl, e.g., tert-butyl, CH₂CCl₃, or para-methoxybenzyl, or a tetrazolyl protecting group such as para-methoxybenzyl or trityl. The numbering used below applies to compounds in Scheme 2. For example, reference to compounds of formula (VII) below encompasses compounds of formulae (VIIb) and (VIIc).

Step 1: Commercially available Grignard reagent (VIII; M=MgBr) or aryllithium (VIII; M=Li)—prepared from the corresponding aryl bromide or iodide—is reacted with cyclobutanone to give compounds of formula (VII).

Step 2: Alcohol (VII) is condensed with compound (IX), wherein X¹ and X² represent leaving groups, such as halo e.g., chloro, bromo or iodo, to give monoester (VI).

Step 3: Monoester (VI) is reacted with a trialkylphosphonoacetate of formula (V), wherein R¹¹ and R¹² independently represent C₁₋₄ alkyl optionally substituted with halo, to provide a compound of formula (IV).

Step 4: Condensation of a compound of formula (IV) with formaldehyde or a formaldehyde equivalent thereof e.g., paraformaldehyde, followed by hydrolysis of the alkyl ester and any optional deprotection steps, provides the compound of formula (III).

Step 5: The acetyl or propanoyl carboxylic acid or tetrazolyl moiety is introduced by reaction of compounds of formula (III) with X—R^{B}—P wherein X represents OH, a leaving group, such as chloro, bromo, iodo, alkanesulfonate, e.g., methanesulfonate, or arenesulfonate, e.g., para-toluenesulfonate or benzenesulfonate, and P represents a carboxylic acid protecting group such as C₁₋₆ alkyl, e.g., tert-butyl, C₁₋₆ haloalkyl, such as CH₂CCl₃, or para-methoxybenzyl or a tetrazolyl protecting group such as para-methoxybenzyl or trityl, suitably para-methoxybenzyl. When X represents a leaving group, the reaction is suitably carried out under basic conditions, such as in the presence of potassium carbonate in dimethylformamide, to give compounds of formula (II). When X is OH, the reaction is suitably carried out using a coupling agent, such as HATU or EDCI, in the presence of a base, such as DIPEA, and a catalyst, e.g., DMAP to give compounds of formula (II). Suitably compounds of formula (III) are coupled with 2,2,2-trichloroethyl 2-bromoacetate or 2,2,2-trichloroethyl 3-hydroxypropanoate. Other carboxyl or tetrazolyl protecting groups may be used and are known to the person skilled in the art, and are described elsewhere herein.

Step 6: Compounds of formula (II) are converted to compounds of formula (I) by removal of P, for example using Zn-mediated reduction or trimethyltin hydroxide in 1,2-dichloroethane when P is CH₂CCl₃, or using acidic conditions when P is C₁₋₆ alkyl, para-methoxybenzyl or trityl.

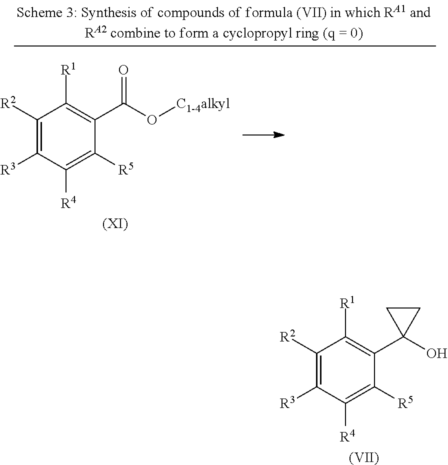

Compounds of formula (VII) in which R^{A1} and R^{A2} combine to form a cyclopropyl ring (q=0) may be prepared from commercially available esters (XI) through a Kulinkovich Reaction using a Grignard reagent suchas EtMgBr and a catalyst such as Ti(OiPr)₄.

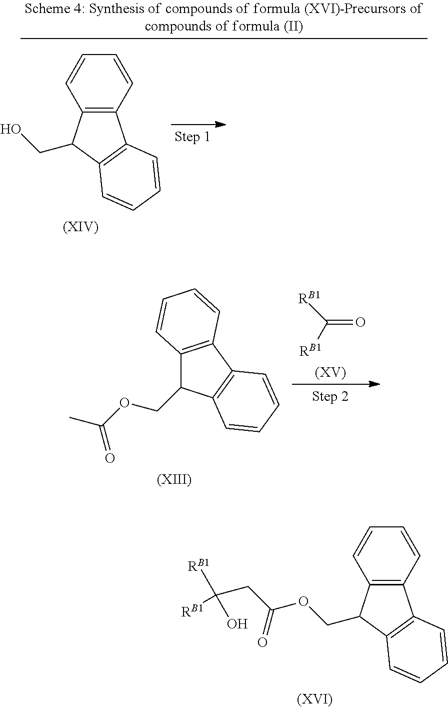

This scheme is particularly useful when R^{B} is C(R^{B1})₂CH₂COOH, i.e., CH₂CH₂COOH substituted on the β carbon atom by two R^{B1} groups, where the two R^{B1} groups are attached to the same carbon atom, and especially where the two R^{B1} groups join to form a C₃₋₆ cycloalkyl ring or a 4-6-membered heterocyclic ring.

Step 1: Esterification of alcohol (XIV) under standard conditions provides esters of formula (XIII).

Step 2: Reaction between esters of formula (XIII) and ketones of formula (XV) under basic conditions (such as using LHMDS) provides compounds of formula (XVI).

In a further step, the compound of formula (XVI) is reacted with a compound of formula (III) to give a compound of formula (II) in which the protecting group P of —R^{BP} is a 9-fluorenylmethoxycarbonyyl (FMOC) group.

The skilled person will appreciate that protecting groups may be used throughout the synthetic schemes described herein to give protected derivatives of any of the above compounds or generic formulae. Protective groups and the means for their removal are described in “Protective Groups in Organic Synthesis”, by Theodora W. Greene and Peter G. M. Wuts, published by John Wiley & Sons Inc; 4th Rev Ed., 2006, ISBN-10: 0471697540. Examples of nitrogen protecting groups include trityl (Tr), tert-butyloxycarbonyl (BOC), 9-fluorenylmethyloxycarbonyl (Fmoc), acetyl (Ac), benzyl (Bn) and para-methoxy benzyl (PMB). Examples of oxygen protecting groups include acetyl (Ac), methoxymethyl (MOM), para-methoxybenzyl (PMB), benzyl, tert-butyl, methyl, ethyl, tetrahydropyranyl (THP), and silyl ethers and esters (such as trimethylsilyl (TMS), tert-butyldimethylsilyl (TBDMS), tri-iso-propylsilyloxymethyl (TOM), and triisopropylsilyl (TIPS) ethers and esters). Specific examples of carboxylic acid protecting groups include trichloroethyl chloroformate (Troc), alkyl esters (such as C₁₋₆ alkyl e.g. C₁₋₄ alkyl esters), benzyl esters and silyl esters. Another example of a carboxylic acid protecting group is 9-fluorenylmethyloxycarbonyl (Fmoc).

The moiety “—R^{B}—P” as used herein means that R^{B} is protected with protecting group P. The location and specific protecting group will depend on the identity of R^{B} which will be understood by the skilled person. The term “—R^{BP}” means the same as “—R^{B}—P”.

For example, when R^{B} comprises CH₂COOH or CH₂CH₂COOH, suitably P is a carboxylic acid protecting group and suitably replaces the hydrogen atom attached to an oxygen atom, i.e., CH₂COO—P or CH₂CH₂COO—P.

When R^{B} comprises CH₂tetrazolyl or CH₂CH₂tetrazolyl, suitably P is a tetrazolyl protecting group which replaces the hydrogen atom attached to a nitrogen atom:

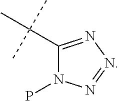

In one embodiment there is provided a process for preparing a compound of formula (I) or a salt such as a pharmaceutically acceptable salt thereof, which comprises reacting a compound of formula (III):

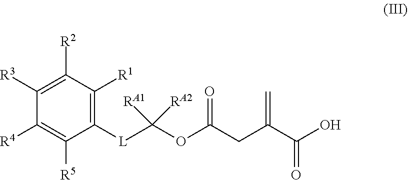

or a salt thereof;
with R^{BP}—X³
wherein R^{BP} is a protected derivative of R^{B}, and X³ is OH or a leaving group such as OMs, OTs or halo such as bromo;
   or a salt thereof;
   followed by deprotection;
   wherein R¹, R², R³, R⁴, R⁵, L, R^{A1}, R^{A2} and R^{B} are as defined elsewhere herein.

Suitably, there is provided a process for preparing a compound of formula (Id) or a salt such as a pharmaceutically acceptable salt thereof, which comprises reacting a compound of formula (IIId):

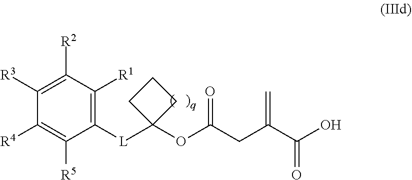

or a salt thereof;
with R^{BP}—X³
wherein R^{BP} is a protected derivative of R^{B}, and X³ is OH or a leaving group such as OMs, OTs or halo such as bromo;
   or a salt thereof;
   followed by deprotection;
   wherein R¹, R², R³, R⁴, R⁵, L, R^{B} and q are as defined elsewhere herein.

There is also provided a process for preparing a compound of formula (Ie) or a salt such as a pharmaceutically acceptable salt thereof, which comprises reacting a compound of formula (Ille):

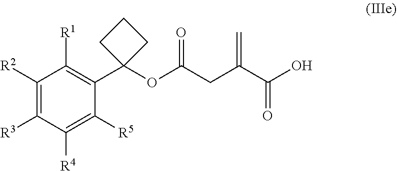

or a salt thereof;
with a compound of formula (Xe):

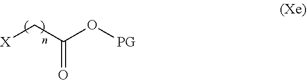

wherein X is OH or a leaving group such as OMs or OTs, or a halo atom such as chloro or bromo and PG is a carboxylic acid protecting group such as tert-butyl, C₁₋₆ haloalkyl, such as CH₂CCl₃, or para-methoxybenzyl, suitably CH₂CCl₃,
or a salt thereof;
followed by removal of the carboxylic acid protecting group;
wherein n, R¹, R², R³, R⁴ and R⁵ are as defined elsewhere herein.

In one embodiment there is provided a process for preparing a compound of formula (Ic) or a salt such as a pharmaceutically acceptable salt thereof, which comprises reacting a compound of formula (IIIc):

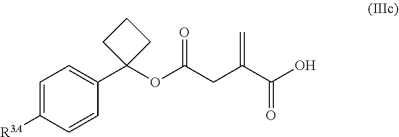

or a salt thereof;
with X—R^{B}—P or a salt thereof;
wherein X is OH, a leaving group, such as OMs or OTs, or a halo atom such as chloro or bromo and P is a carboxylic acid protecting group such as C₁₋₆ alkyl e.g., tert-butyl, C₁₋₆ haloalkyl, such as CH₂CCl₃, or para-methoxybenzyl, or a tetrazolyl protecting group such as para-methoxybenzyl or trityl,
or a salt thereof;
followed by removal of P;
wherein R^{3A} and R^{B} are as defined elsewhere herein.

In one embodiment, there is provided a compound of formula (II):

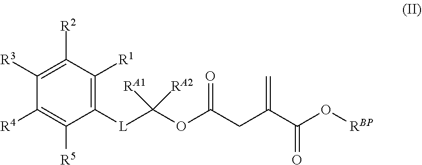

wherein R¹, R², R³, R⁴, R⁵, L, R^{A1}, R^{A2} and R^{BP} are as defined elsewhere herein,
or a salt thereof.

Suitably, the compound of formula (II) is:

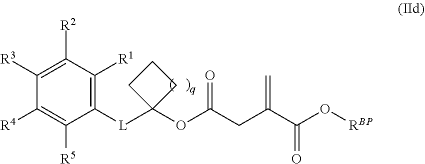

wherein R¹, R², R³, R⁴, R⁵, L, q and R^{BP} are as defined elsewhere herein,
or a salt thereof.

Suitably, the compound of formula (II) is:

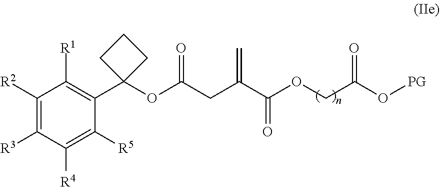

wherein n, R¹, R², R³, R⁴, R⁵ and PG are as defined elsewhere herein,
or a salt thereof.

Suitably, the compound of formula (II) is:

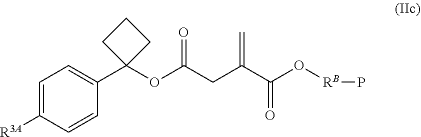

wherein R^{3A} and R^{B} are as defined elsewhere herein, and P is a carboxylic acid protecting group such as C₁₋₆ alkyl e.g., tert-butyl, C₁₋₆ haloalkyl, e.g., CH₂CCl₃, or para-methoxybenzyl, or a tetrazolyl protecting group, such as para-methoxybenzyl or trityl;
or a salt thereof.

In one embodiment, there is provided a compound of formula (III):

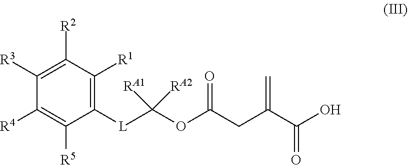

wherein R¹, R², R³, R⁴, R⁵, L, R^{A1} and R^{A2} are as defined elsewhere herein,
or a salt thereof.

Suitably, the compound of formula (III) is:

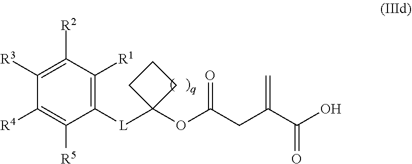

wherein R¹, R², R³, R⁴, R⁵, L and q are as defined elsewhere herein,
or a salt thereof.

Suitably, the compound of formula (III) is:

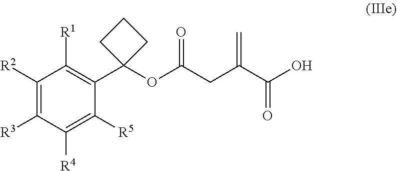

wherein R¹, R², R³, R⁴ and R⁵ are as defined elsewhere herein,
or a salt thereof.

Suitably, the compound of formula (III) is:

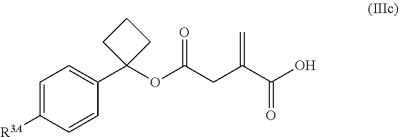

wherein R^{3A} is as defined elsewhere herein,
or a salt thereof.

In one embodiment, there is provided a compound of formula (IV):

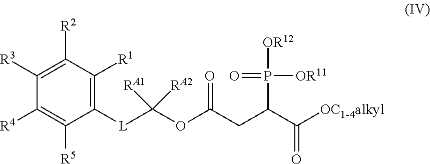

wherein R¹, R², R³, R⁴, R⁵, L, R^{A1}, R^{A2}, R¹¹ and R¹² are as defined elsewhere herein,
or a salt thereof.

Suitably, the compound of formula (IV) is:

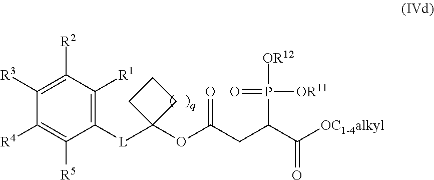

wherein R¹, R², R³, R⁴, R⁵, L, q, R¹¹ and R¹² are as defined elsewhere herein,
or a salt thereof.

Suitably, the compound of formula (IV) is:

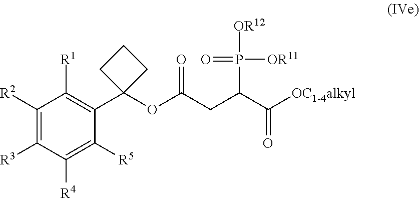

wherein R¹, R², R³, R⁴, R⁵, R¹¹ and R¹² are as defined elsewhere herein,
or a salt thereof.

Suitably, the compound of formula (IV) is:

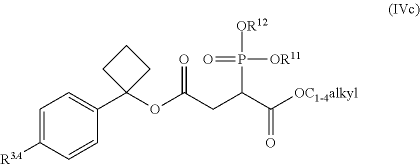

wherein R^{3A}, R¹¹ and R¹² are as defined elsewhere herein,
or a salt thereof.

In one embodiment, there is provided a compound of formula (VI):

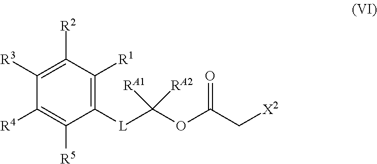

wherein R¹, R², R³, R⁴, R⁵, L, R^{A1}, R^{A2} and X² are as defined elsewhere herein,
or a salt thereof.

Suitably, the compound of formula (VI) is:

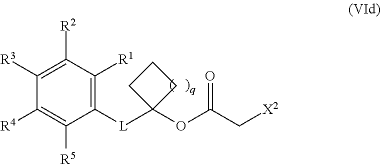

wherein R¹, R², R³, R⁴, R⁵, L, q and X² are as defined elsewhere herein,
or a salt thereof.

Suitably, the compound of formula (VI) is:

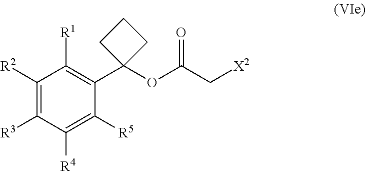

wherein R¹, R², R³, R⁴, R⁵ and X² are as defined elsewhere herein,
or a salt thereof.

Suitably, the compound of formula (VI) is:

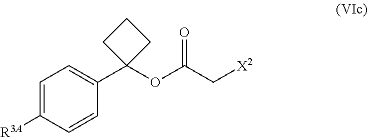

wherein R^{3A} is as defined elsewhere herein, and X² is a leaving group, such as halo e.g., chloro, bromo or iodo,
or a salt thereof.

In one embodiment, there is provided a compound of formula (XVI):

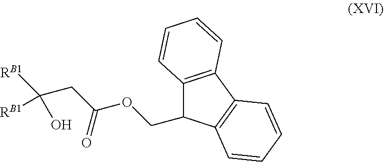

wherein R^{B1} is as defined elsewhere herein;
or a salt thereof.

Certain intermediates are novel and are claimed as an aspect of the invention:
4-(1-(3,5-dichlorophenyl)cyclobutyl) 1-methyl 2-(diethoxyphosphoryl)succinate;
4-(1-(3,5-dichlorophenyl)cyclobutyl) 1-methyl 2-methylenesuccinate;
4-(1-(3,5-dichlorophenyl)cyclobutoxy)-2-methylene-4-oxobutanoic acid;
4-(1-(3,5-dichlorophenyl)cyclobutyl) 1-(2-oxo-2-(2,2,2-trichloroethoxy)ethyl) 2-methylenesuccinate;
1-methyl 4-(1-(4-(trifluoromethyl)phenyl)cyclobutyl) 2-(diethoxyphosphoryl)succinate;
1-methyl 4-(1-(4-(trifluoromethyl)phenyl)cyclobutyl) 2-methylenesuccinate;
2-methylene-4-oxo-4-(1-(4-(trifluoromethyl)phenyl)cyclobutoxy)butanoic acid;
1-(2-oxo-2-(2,2,2-trichloroethoxy)ethyl) 4-(1-(4-(trifluoromethyl)phenyl)cyclobutyl) 2-methylenesuccinate;
   or salts thereof.

Further intermediates are novel and are claimed as an aspect of the invention:
2-methylene-4-oxo-4-(1-(4-((trifluoromethyl)thio)phenyl)cyclobutoxy)butanoic acid;
1-(2-oxo-2-(2,2,2-trichloroethoxy)ethyl) 4-(1-(4-((trifluoromethyl)thio)phenyl)cyclobutyl) 2-methylenesuccinate;
1-(3-oxo-3-(2,2,2-trichloroethoxy)propyl) 4-(1-(4-((trifluoromethyl)thio)phenyl)cyclobutyl) 2-methylenesuccinate;
2-methylene-4-oxo-4-(1-(4-(pentafluoro-λ⁶-sulfaneyl)phenyl)cyclobutoxy)butanoic acid;
1-(2-oxo-2-(2,2,2-trichloroethoxy)ethyl) 4-(1-(4-(pentafluoro-λ⁶-sulfaneyl)phenyl)cyclobutyl) 2-methylenesuccinate; and
1-(3-oxo-3-(2,2,2-trichloroethoxy)propyl) 4-(1-(4-(pentafluoro-λ⁶-sulfaneyl)phenyl)cyclobutyl) 2-methylenesuccinate;
   or salts thereof.

Suitably, the novel intermediates are:
2-methylene-4-oxo-4-(1-(4-((trifluoromethyl)thio)phenyl)cyclobutoxy)butanoic acid;
1-(2-oxo-2-(2,2,2-trichloroethoxy)ethyl) 4-(1-(4-((trifluoromethyl)thio)phenyl)cyclobutyl) 2-methylenesuccinate; and
1-(3-oxo-3-(2,2,2-trichloroethoxy)propyl) 4-(1-(4-((trifluoromethyl)thio)phenyl)cyclobutyl) 2-methylenesuccinate;
   or salts thereof.

Further intermediates are novel and are claimed as an aspect of the invention:
2,2,2-trichloroethyl 3-hydroxypropanoate; and
(9H-fluoren-9-yl)methyl 2-(3-hydroxyoxetan-3-yl)acetate;
   or salts thereof.

It will be appreciated that for use in therapy the salts of the compounds of formula (I) should be pharmaceutically acceptable. Suitable pharmaceutically acceptable salts will be apparent to those skilled in the art. Pharmaceutically acceptable salts include acid addition salts, suitably salts of compounds of the invention comprising a basic group such as an amino group, formed with inorganic acids e.g. hydrochloric acid, hydrobromic acid, sulfuric acid, nitric acid or phosphoric acid. Also included are salts formed with organic acids e.g. succinic acid, maleic acid, acetic acid, fumaric acid, citric acid, tartaric acid, benzoic acid, p-toluenesulfonic acid, methanesulfonic acid, naphthalenesulfonic acid and 1,5-naphthalenedisulfonic acid. Other salts e.g. oxalates or formates, may be used, for example in the isolation of compounds of formula (I) and are included within the scope of this invention, as are basic addition salts such as sodium, potassium, calcium, aluminium, zinc, magnesium and other metal salts.

Pharmaceutically acceptable salts may also be formed with organic bases such as basic amines e.g. with ammonia, meglumine, tromethamine, piperazine, arginine, choline, diethylamine, benzathine or lysine. Thus, in one embodiment there is provided a compound of formula (I) in the form of a pharmaceutically acceptable salt. Alternatively, there is provided a compound of formula (I) in the form of a free acid. When the compound contains a basic group as well as the free acid it may be Zwitterionic.

Suitably, the compound of formula (I) is not a salt e.g. is not a pharmaceutically acceptable salt.

Suitably, where the compound of formula (I) is in the form of a salt, the pharmaceutically acceptable salt is a basic addition salt such as a carboxylate salt formed with a group 1 metal (e.g. a sodium or potassium salt), a group 2 metal (e.g. a magnesium or calcium salt) or an ammonium salt of a basic amine (e.g. an NH₄⁺ salt), such as a sodium salt.

The compounds of formula (I) may be prepared in crystalline or non-crystalline form and, if crystalline, may optionally be solvated, e.g. as the hydrate. This invention includes within its scope stoichiometric solvates (e.g. hydrates) as well as compounds containing variable amounts of solvent (e.g. water). Suitably, the compound of formula (I) is not a solvate.

The invention extends to a pharmaceutically acceptable derivative thereof, such as a pharmaceutically acceptable prodrug of compounds of formula (I). Typical prodrugs of compounds of formula (I) which comprise a carboxylic acid include ester (e.g. C₁₋₆alkyl e.g. C₁₋₄ alkyl ester) derivatives thereof. Thus, in one embodiment, the compound of formula (I) is provided as a pharmaceutically acceptable prodrug. In another embodiment, the compound of formula (I) is not provided as a pharmaceutically acceptable prodrug.

Certain compounds of formula (I) may metabolise under certain conditions such as by hydrolysis of the α,β-unsaturated ester group. Without wishing to be bound by theory, formation of an active metabolite (such as in vivo) of a compound of formula (I) may be beneficial by contributing to the biological activity observed of the compound of formula (I). Thus, in one embodiment, there is provided an active metabolite of the compound of formula (I) and its use as a pharmaceutical e.g. for the treatment or prevention of the diseases mentioned herein.

It is to be understood that the present invention encompasses all isomers of compounds of formula (I) including all geometric, tautomeric and optical forms, and mixtures thereof (e.g. racemic mixtures). Where additional chiral centres are present in compounds of formula (I), the present invention includes within its scope all possible diastereoisomers, including mixtures thereof. The different isomeric forms may be separated or resolved one from the other by conventional methods, or any given isomer may be obtained by conventional synthetic methods or by stereospecific or asymmetric syntheses.

The present invention also includes all isotopic forms of the compounds provided herein, whether in a form (i) wherein all atoms of a given atomic number have a mass number (or mixture of mass numbers) which predominates in nature (referred to herein as the “natural isotopic form”) or (ii) wherein one or more atoms are replaced by atoms having the same atomic number, but a mass number different from the mass number of atoms which predominates in nature (referred to herein as an “unnatural variant isotopic form”). It is understood that an atom may naturally exist as a mixture of mass numbers. The term “unnatural variant isotopic form” also includes embodiments in which the proportion of an atom of given atomic number having a mass number found less commonly in nature (referred to herein as an “uncommon isotope”) has been increased relative to that which is naturally occurring e.g. to the level of >20%, >50%, >75%, >90%, >95% or >99% by number of the atoms of that atomic number (the latter embodiment referred to as an “isotopically enriched variant form”). The term “unnatural variant isotopic form” also includes embodiments in which the proportion of an uncommon isotope has been reduced relative to that which is naturally occurring. Isotopic forms may include radioactive forms (i.e. they incorporate radioisotopes) and non-radioactive forms. Radioactive forms will typically be isotopically enriched variant forms.

An unnatural variant isotopic form of a compound may thus contain one or more artificial or uncommon isotopes such as deuterium (²H or D), carbon-11 (¹¹C), carbon-13 (¹³C), carbon-14 (¹⁴C), nitrogen-13 (¹³N), nitrogen-15 (¹⁵N), oxygen-15 (¹⁵O), oxygen-17 (¹⁷O), oxygen-18 (¹⁸O), phosphorus-32 (³²P), sulphur-35 (³⁵S), chlorine-36 (³⁶Cl), chlorine-37 (³⁷Cl), fluorine-18 (¹⁸F) iodine-123 (¹²³I), iodine-125 (¹²⁵I) in one or more atoms or may contain an increased proportion of said isotopes as compared with the proportion that predominates in nature in one or more atoms.

Unnatural variant isotopic forms comprising radioisotopes may, for example, be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Unnatural variant isotopic forms which incorporate deuterium i.e. ²H or D may afford certain therapeutic advantages resulting from greater metabolic stability, for example, increased in vivo half-life or reduced dosage requirements, and hence may be preferred in some circumstances. Further, unnatural variant isotopic forms may be prepared which incorporate positron emitting isotopes, such as ¹¹C, ¹⁸F, ¹⁵O and ¹³N, and would be useful in positron emission topography (PET) studies for examining substrate receptor occupancy.

In one embodiment, the compounds of formula (I) are provided in a natural isotopic form. In one embodiment, the compounds of formula (I) are provided in an unnatural variant isotopic form. In a specific embodiment, the unnatural variant isotopic form is a form in which deuterium (i.e. ²H or D) is incorporated where hydrogen is specified in the chemical structure in one or more atoms of a compound of formula (I). In one embodiment, the atoms of the compounds of formula (I) are in an isotopic form which is not radioactive. In one embodiment, one or more atoms of the compounds of formula (I) are in an isotopic form which is radioactive. Suitably radioactive isotopes are stable isotopes. Suitably the unnatural variant isotopic form is a pharmaceutically acceptable form.

In one embodiment, a compound of formula (I) is provided whereby a single atom of the compound exists in an unnatural variant isotopic form. In another embodiment, a compound of formula (I) is provided whereby two or more atoms exist in an unnatural variant isotopic form.

Unnatural isotopic variant forms can generally be prepared by conventional techniques known to those skilled in the art or by processes described herein e.g. processes analogous to those described in the accompanying Examples for preparing natural isotopic forms. Thus, unnatural isotopic variant forms could be prepared by using appropriate isotopically variant (or labelled) reagents in place of the normal reagents employed in the Examples. Since the compounds of formula (I) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

Therapeutic Indications

Compounds of formula (I) are of use in therapy, particularly for treating or preventing an inflammatory disease or a disease associated with an undesirable immune response. As shown in Biological Example 1 below, example compounds of formula (I) reduced cytokine release more effectively than dimethyl itaconate, as demonstrated by lower IC₅₀ values in at least the IL-1β assay. Cytokines are important mediators of inflammation and immune-mediated disease as evidenced by the therapeutic benefit delivered by antibodies targeting them.

Thus, in a first aspect, the present invention provides a compound of formula (I) or a pharmaceutically acceptable salt and/or solvate thereof as defined herein, for use as a medicament. Also provided is a pharmaceutical composition comprising a compound of formula (I) or a pharmaceutically acceptable salt and/or solvate thereof as defined herein. Such a pharmaceutical composition contains the compound of formula (I) and a pharmaceutically acceptable carrier or excipient.

In a further aspect, the present invention provides a compound of formula (I) or a pharmaceutically acceptable salt and/or solvate thereof as defined herein, for use in treating or preventing an inflammatory disease or a disease associated with an undesirable immune response. In a further aspect, the present invention provides the use of a compound of formula (I) or a pharmaceutically acceptable salt and/or solvate thereof as defined herein, in the manufacture of a medicament for treating or preventing an inflammatory disease or a disease associated with an undesirable immune response. In a further aspect, the present invention provides a method of treating or preventing an inflammatory disease or a disease associated with an undesirable immune response, which comprises administering a compound of formula (I) or a pharmaceutically acceptable salt and/or solvate thereof as defined herein.

For all aspects of the invention, suitably the compound is administered to a subject in need thereof, wherein the subject is suitably a human subject.

In one embodiment is provided a compound of formula (I) or a pharmaceutically acceptable salt and/or solvate thereof as defined herein, for use in treating an inflammatory disease or disease associated with an undesirable immune response. In one embodiment of the invention is provided the use of a compound of formula (I) or a pharmaceutically acceptable salt and/or solvate thereof as defined herein, in the manufacture of a medicament for treating an inflammatory disease or a disease associated with an undesirable immune response. In one embodiment of the invention is provided a method of treating an inflammatory disease or a disease associated with an undesirable immune response, which comprises administering a compound of formula (I) or a pharmaceutically acceptable salt and/or solvate thereof as defined herein.

In one embodiment is provided a compound of formula (I) or a pharmaceutically acceptable salt and/or solvate thereof as defined herein, for use in preventing an inflammatory disease or a disease associated with an undesirable immune response. In one embodiment of the invention is provided the use of a compound of formula (I) or a pharmaceutically acceptable salt and/or solvate thereof as defined herein, in the manufacture of a medicament for preventing an inflammatory disease or a disease associated with an undesirable immune response. In one embodiment of the invention is provided a method of preventing an inflammatory disease or a disease associated with an undesirable immune response, which comprises administering a compound of formula (I) or a pharmaceutically acceptable salt and/or solvate thereof as defined herein.

In one embodiment is provided a compound of formula (I) or a pharmaceutically acceptable salt and/or solvate thereof as defined herein, for use in treating or preventing an inflammatory disease. In one embodiment of the invention is provided the use of a compound of formula (I) or a pharmaceutically acceptable salt and/or solvate thereof as defined herein, in the manufacture of a medicament for treating or preventing an inflammatory disease. In one embodiment of the invention is provided a method of treating or preventing an inflammatory disease, which comprises administering a compound of formula (I) or a pharmaceutically acceptable salt and/or solvate thereof as defined herein.

In one embodiment is provided a compound of formula (I) or a pharmaceutically acceptable salt and/or solvate thereof as defined herein, for use in treating or preventing a disease associated with an undesirable immune response. In one embodiment of the invention is provided the use of a compound of formula (I) or a pharmaceutically acceptable salt and/or solvate thereof as defined herein, in the manufacture of a medicament for treating or preventing a disease associated with an undesirable immune response. In one embodiment of the invention is provided a method of treating or preventing a disease associated with an undesirable immune response, which comprises administering a compound of formula (I) or a pharmaceutically acceptable salt and/or solvate thereof as defined herein.

An undesirable immune response will typically be an immune response which gives rise to a pathology i.e. is a pathological immune response or reaction.

In one embodiment, the inflammatory disease or disease associated with an undesirable immune response is an auto-immune disease.

In one embodiment, the inflammatory disease or disease associated with an undesirable immune response is, or is associated with, a disease selected from the group consisting of: psoriasis (including chronic plaque, erythrodermic, pustular, guttate, inverse and nail variants), asthma, chronic obstructive pulmonary disease (COPD, including chronic bronchitis and emphysema), heart failure (including left ventricular failure), myocardial infarction, angina pectoris, other atherosclerosis and/or atherothrombosis-related disorders (including peripheral vascular disease and ischaemic stroke), a mitochondrial and neurodegenerative disease (such as Parkinson's disease, Alzheimer's disease, Huntington's disease, amyotrophic lateral sclerosis, retinitis pigmentosa or mitochondrial encephalomyopathy), autoimmune paraneoplastic retinopathy, transplantation rejection (including antibody-mediated and T cell-mediated forms), multiple sclerosis, transverse myelitis, ischaemia-reperfusion injury (e.g. during elective surgery such as cardiopulmonary bypass for coronary artery bypass grafting or other cardiac surgery, following percutaneous coronary intervention, following treatment of acute ST-elevation myocardial infarction or ischaemic stroke, organ transplantation, or acute compartment syndrome), AGE-induced genome damage, an inflammatory bowel disease (e.g. Crohn's disease or ulcerative colitis), primary sclerosing cholangitis (PSC), PSC-autoimmune hepatitis overlap syndrome, non-alcoholic fatty liver disease (non-alcoholic steatohepatitis), rheumatica, granuloma annulare, cutaneous lupus erythematosus (CLE), systemic lupus erythematosus (SLE), lupus nephritis, drug-induced lupus, autoimmune myocarditis or myopericarditis, Dressler's syndrome, giant cell myocarditis, post-pericardiotomy syndrome, drug-induced hypersensitivity syndromes (including hypersensitivity myocarditis), eczema, sarcoidosis, erythema nodosum, acute disseminated encephalomyelitis (ADEM), neuromyelitis optica spectrum disorders, MOG (myelin oligodendrocyte glycoprotein) antibody-associated disorders (including MOG-EM), optic neuritis, CLIPPERS (chronic lymphocytic inflammation with pontine perivascular enhancement responsive to steroids), diffuse myelinoclastic sclerosis, Addison's disease, alopecia areata, ankylosing spondylitis, other spondyloarthritides (including peripheral spondyloarthritis, that is associated with psoriasis, inflammatory bowel disease, reactive arthritis or juvenile onset forms), antiphospholipid antibody syndrome, autoimmune hemolytic anaemia, autoimmune hepatitis, autoimmune inner ear disease, pemphigoid (including bullous pemphigoid, mucous membrane pemphigoid, cicatricial pemphigoid, herpes gestationis or pemphigoid gestationis, ocular cicatricial pemphigoid), linear IgA disease, Behçet's disease, celiac disease, Chagas disease, dermatomyositis, diabetes mellitus type I, endometriosis, Goodpasture's syndrome, Graves' disease, Guillain-Barre syndrome and its subtypes (including acute inflammatory demyelinating polyneuropathy, AIDP, acute motor axonal neuropathy (AMAN), acute motor and sensory axonal neuropathy (AMSAN), pharyngeal-cervical-brachial variant, Miller-Fisher variant and Bickerstaff's brainstem encephalitis), progressive inflammatory neuropathy, Hashimoto's disease, hidradenitis suppurativa, inclusion body myositis, necrotising myopathy, Kawasaki disease, IgA nephropathy, Henoch-Schonlein purpura, idiopathic thrombocytopenic purpura, thrombotic thrombocytopenic purpura (TTP), Evans' syndrome, interstitial cystitis, mixed connective tissue disease, undifferentiated connective tissue disease, morphea, myasthenia gravis (including MuSK antibody positive and seronegative variants), narcolepsy, neuromyotonia, pemphigus vulgaris, pernicious anaemia, psoriatic arthritis, polymyositis, primary biliary cholangitis (also known as primary biliary cirrhosis), rheumatoid arthritis, palindromic rheumatism, schizophrenia, autoimmune (meningo-)encephalitis syndromes, scleroderma, Sjogren's syndrome, stiff person syndrome, polymylagia rheumatica, giant cell arteritis (temporal arteritis), Takayasu arteritis, polyarteritis nodosa, Kawasaki disease, granulomatosis with polyangitis (GPA; formerly known as Wegener's granulomatosis), eosinophilic granulomatosis with polyangiitis (EGPA; formerly known as Churg-Strauss syndrome), microscopic polyarteritis/polyangiitis, hypocomplementaemic urticarial vasculitis, hypersensitivity vasculitis, cryoglobulinemia, thromboangiitis obliterans (Buerger's disease), vasculitis, leukocytoclastic vasculitis, vitiligo, acute disseminated encephalomyelitis, adrenoleukodystrophy, Alexander's disease, Alper's disease, balo concentric sclerosis or Marburg disease, cryptogenic organising pneumonia (formerly known as bronchiolitis obliterans organizing pneumonia), Canavan disease, central nervous system vasculitic syndrome, Charcot-Marie-Tooth disease, childhood ataxia with central nervous system hypomyelination, chronic inflammatory demyelinating polyneuropathy (CIDP), diabetic retinopathy, globoid cell leukodystrophy (Krabbe disease), graft-versus-host disease (GVHD) (including acute and chronic forms, as well as intestinal GVHD), hepatitis C (HCV) infection or complication, herpes simplex viral infection or complication, human immunodeficiency virus (HIV) infection or complication, lichen planus, monomelic amyotrophy, cystic fibrosis, pulmonary arterial hypertension (PAH, including idiopathic PAH), lung sarcoidosis, idiopathic pulmonary fibrosis, paediatric asthma, atopic dermatitis, allergic dermatitis, contact dermatitis, allergic rhinitis, rhinitis, sinusitis, conjunctivitis, allergic conjunctivitis, keratoconjunctivitis sicca, dry eye, xerophthalmia, glaucoma, macular oedema, diabetic macular oedema, central retinal vein occlusion (CRVO), macular degeneration (including dry and/or wet age related macular degeneration, AMD), post-operative cataract inflammation, uveitis (including posterior, anterior, intermediate and pan uveitis), iridocyclitis, scleritis, corneal graft and limbal cell transplant rejection, gluten sensitive enteropathy (coeliac disease), dermatitis herpetiformis, eosinophilic esophagitis, achalasia, autoimmune dysautonomia, autoimmune encephalomyelitis, autoimmune oophoritis, autoimmune orchitis, autoimmune pancreatitis, aortitis and periaortitis, autoimmune retinopathy, autoimmune urticaria, Behcet's disease, (idiopathic) Castleman's disease, Cogan's syndrome, IgG4-related disease, retroperitoneal fibrosis, juvenile idiopathic arthritis including systemic juvenile idiopathic arthritis (Still's disease), adult-onset Still's disease, ligneous conjunctivitis, Mooren's ulcer, pityriasis lichenoides et varioliformis acuta (PLEVA, also known as Mucha-Habermann disease), multifocal motor neuropathy (MMN), paediatric acute-onset neuropsychiatric syndrome (PANS) (including paediatric autoimmune neuropsychiatric disorders associated with streptococcal infections (PANDAS)), paraneoplastic syndromes (including paraneoplastic cerebellar degeneration, Lambert-Eaton myaesthenic syndrome, limbic encephalitis, brainstem encephalitis, opsoclonus myoclonus ataxia syndrome, anti-NMDA receptor encephalitis, thymoma-associated multiorgan autoimmunity), perivenous encephalomyelitis, reflex sympathetic dystrophy, relapsing polychondritis, sperm & testicular autoimmunity, Susac's syndrome, Tolosa-Hunt syndrome, Vogt-Koyanagi-Harada Disease, anti-synthetase syndrome, autoimmune enteropathy, immune dysregulation polyendocrinopathy enteropathy X-linked (IPEX), microscopic colitis, autoimmune lymphoproliferative syndrome (ALPS), autoimmune polyendocrinopathy-candidiasis-ectodermal dystrophy syndrome (APEX), gout, pseudogout, amyloid (including AA or secondary amyloidosis), eosinophilic fasciitis (Shulman syndrome) progesterone hypersensitivity (including progesterone dermatitis), familial Mediterranean fever (FMF), tumour necrosis factor (TNF) receptor-associated periodic fever syndrome (TRAPS), hyperimmunoglobulinaemia D with periodic fever syndrome (HIDS), PAPA (pyogenic arthritis, pyoderma gangrenosum, severe cystic acne) syndrome, deficiency of interleukin-1 receptor antagonist (DIRA), deficiency of the interleukin-36-receptor antagonist (DITRA), cryopyrin-associated periodic syndromes (CAPS) (including familial cold autoinflammatory syndrome [FCAS], Muckle-Wells syndrome, neonatal onset multisystem inflammatory disease [NOMID]), NLRP12-associated autoinflammatory disorders (NLRP12AD), periodic fever aphthous stomatitis (PFAPA), chronic atypical neutrophilic dermatosis with lipodystrophy and elevated temperature (CANDLE), Majeed syndrome, Blau syndrome (also known as juvenile systemic granulomatosis), macrophage activation syndrome, chronic recurrent multifocal osteomyelitis (CRMO), familial cold autoinflammatory syndrome, mutant adenosine deaminase 2 and monogenic interferonopathies (including Aicardi-Goutières syndrome, retinal vasculopathy with cerebral leukodystrophy, spondyloenchondrodysplasia, STING [stimulator of interferon genes]-associated vasculopathy with onset in infancy, proteasome associated autoinflammatory syndromes, familial chilblain lupus, dyschromatosis symmetrica hereditaria), Schnitzler syndrome; familial cylindromatosis, congenital B cell lymphocytosis, OTULIN-related autoinflammatory syndrome, type 2 diabetes mellitus, insulin resistance and the metabolic syndrome (including obesity-associated inflammation), atherosclerotic disorders (e.g. myocardial infarction, angina, ischaemic heart failure, ischaemic nephropathy, ischaemic stroke, peripheral vascular disease, aortic aneurysm), renal inflammatory disorders (e.g. diabetic nephropathy, membranous nephropathy, minimal change disease, crescentic glomerulonephritis, acute kidney injury, renal transplantation).

In one embodiment, the inflammatory disease or disease associated with an undesirable immune response is, or is associated with, a disease selected from the following autoinflammatory diseases: familial Mediterranean fever (FMF), tumour necrosis factor (TNF) receptor-associated periodic fever syndrome (TRAPS), hyperimmunoglobulinaemia D with periodic fever syndrome (HIDS), PAPA (pyogenic arthritis, pyoderma gangrenosum, and severe cystic acne) syndrome, deficiency of interleukin-1 receptor antagonist (DIRA), deficiency of the interleukin-36-receptor antagonist (DITRA), cryopyrin-associated periodic syndromes (CAPS) (including familial cold autoinflammatory syndrome [FCAS], Muckle-Wells syndrome, and neonatal onset multisystem inflammatory disease [NOMID]), NLRP12-associated autoinflammatory disorders (NLRP12AD), periodic fever aphthous stomatitis (PFAPA), chronic atypical neutrophilic dermatosis with lipodystrophy and elevated temperature (CANDLE), Majeed syndrome, Blau syndrome (also known as juvenile systemic granulomatosis), macrophage activation syndrome, chronic recurrent multifocal osteomyelitis (CRMO), familial cold autoinflammatory syndrome, mutant adenosine deaminase 2 and monogenic interferonopathies (including Aicardi-Goutières syndrome, retinal vasculopathy with cerebral leukodystrophy, spondyloenchondrodysplasia, STING [stimulator of interferon genes]-associated vasculopathy with onset in infancy, proteasome associated autoinflammatory syndromes, familial chilblain lupus, dyschromatosis symmetrica hereditaria) and Schnitzler syndrome.

In one embodiment, the inflammatory disease or disease associated with an undesirable immune response is, or is associated with, a disease selected from the following diseases mediated by excess NF-κB or gain of function in the NF-κB signalling pathway or in which there is a major contribution to the abnormal pathogenesis therefrom (including non-canonical NF-κB signalling): familial cylindromatosis, congenital B cell lymphocytosis, OTULIN-related autoinflammatory syndrome, type 2 diabetes mellitus, insulin resistance and the metabolic syndrome (including obesity-associated inflammation), atherosclerotic disorders (e.g. myocardial infarction, angina, ischaemic heart failure, ischaemic nephropathy, ischaemic stroke, peripheral vascular disease, aortic aneurysm), renal inflammatory disorders (e.g. diabetic nephropathy, membranous nephropathy, minimal change disease, crescentic glomerulonephritis, acute kidney injury, renal transplantation), asthma, COPD, type 1 diabetes mellitus, rheumatoid arthritis, multiple sclerosis, inflammatory bowel disease (including ulcerative colitis and Crohn's disease), and SLE.

In one embodiment, the disease is selected from the group consisting of rheumatoid arthritis, psoriatic arthritis, ankylosing spondylitis, systemic lupus erythematosus, multiple sclerosis, psoriasis, Crohn's disease, ulcerative colitis, uveitis, cryopyrin-associated periodic syndromes, Muckle-Wells syndrome, juvenile idiopathic arthritis and chronic obstructive pulmonary disease.

In one embodiment, the disease is multiple sclerosis.

In one embodiment, the disease is psoriasis.

In one embodiment, the disease is asthma.

In one embodiment, the disease is chronic obstructive pulmonary disease.

In one embodiment, the disease is systemic lupus erythematosus.

In one embodiment, the compound of formula (I) exhibits a lower IC₅₀ compared with dimethyl itaconate when tested in a cytokine assay e.g. as described in Biological Example 1.

In one embodiment, the compound of formula (I) exhibits a lower EC₅₀ compared with dimethyl itaconate when tested in an NRF2 assay e.g. as described in Biological Example 2. In one embodiment, the compound of formula (I) exhibits a higher Eₘₐₓ compared with dimethyl itaconate when tested in an NRF2 assay e.g. as described in Biological Example 2. In one embodiment, the compound of formula (I) exhibits a lower EC₅₀ and/or higher Eₘₐₓ compared with dimethyl itaconate when tested in an NRF2 assay e.g. as described in Biological Example 2. In one embodiment, the compound of formula (I) exhibits a lower EC₅₀ and higher Eₘₐₓ compared with dimethyl itaconate when tested in an NRF2 assay e.g. as described in Biological Example 2.

In one embodiment, the compound of formula (I) exhibits lower intrinsic clearance (Clᵢₙₜ) compared with 4-octyl itaconate when tested in a hepatocyte stability assay (such as in human hepatocyates), e.g., as described in Biological Example 3. In one embodiment, the compound of formula (I) exhibits a longer half-life (T_{1/2}) compared with 4-octyl itaconate when tested in a hepatocyte stability assay (such as in human hepatocyates), e.g. as described in Biological Example 3.

Administration

The compound of formula (I) is usually administered as a pharmaceutical composition. Thus, in one embodiment, is provided a pharmaceutical composition comprising a compound of formula (I) and one or more pharmaceutically acceptable diluents or carriers.

The compound of formula (I) may be administered by any convenient method, e.g. by oral, parenteral, buccal, sublingual, nasal, rectal, intrathecal or transdermal administration, and the pharmaceutical compositions adapted accordingly.

The compound of formula (I) may be administered topically to the target organ e.g. topically to the eye, lung, nose or skin. Hence the invention provides a pharmaceutical composition comprising a compound of formula (I) optionally in combination with one or more topically acceptable diluents or carriers.

A compound of formula (I) which is active when given orally can be formulated as a liquid or solid, e.g. as a syrup, suspension, emulsion, tablet, capsule or lozenge.

A liquid formulation will generally consist of a suspension or solution of the compound of formula (I) in a suitable liquid carrier(s). Suitably the carrier is non-aqueous e.g. polyethylene glycol or an oil. The formulation may also contain a suspending agent, preservative, flavouring and/or colouring agent.

A composition in the form of a tablet can be prepared using any suitable pharmaceutical carrier(s) routinely used for preparing solid formulations, such as magnesium stearate, starch, lactose, sucrose and cellulose.

A composition in the form of a capsule can be prepared using routine encapsulation procedures, e.g. pellets containing the active ingredient can be prepared using standard carriers and then filled into a hard gelatine capsule; alternatively, a dispersion or suspension can be prepared using any suitable pharmaceutical carrier(s), e.g. aqueous gums, celluloses, silicates or oils and the dispersion or suspension then filled into a soft gelatine capsule.

Typical parenteral compositions consist of a solution or suspension of the compound of formula (I) in a sterile aqueous carrier or parenterally acceptable oil, e.g. polyethylene glycol, polyvinyl pyrrolidone, lecithin, arachis oil or sesame oil. Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the compound of formula (I) in a pharmaceutically acceptable aqueous or non-aqueous solvent and are usually presented in single or multidose quantities in sterile form in a sealed container which can take the form of a cartridge or refill for use with an atomising device. Alternatively, the sealed container may be a disposable dispensing device such as a single dose nasal inhaler or an aerosol dispenser fitted with a metering valve. Where the dosage form comprises an aerosol dispenser, it will contain a propellant which can be a compressed gas e.g. air, or an organic propellant such as a chlorofluorocarbon (CFC) or a hydrofluorocarbon (HFC). Aerosol dosage forms can also take the form of pump-atomisers.

Topical administration to the lung may be achieved by use of an aerosol formulation. Aerosol formulations typically comprise the active ingredient suspended or dissolved in a suitable aerosol propellant, such as a chlorofluorocarbon (CFC) or a hydrofluorocarbon (HFC).

Topical administration to the lung may also be achieved by use of a non-pressurised formulation such as an aqueous solution or suspension. These may be administered by means of a nebuliser e.g. one that can be hand-held and portable or for home or hospital use (i.e. non-portable). The formulation may comprise excipients such as water, buffers, tonicity adjusting agents, pH adjusting agents, surfactants and co-solvents.

Topical administration to the lung may also be achieved by use of a dry-powder formulation. The formulation will typically contain a topically acceptable diluent such as lactose, glucose or mannitol (preferably lactose).

The compound of the invention may also be administered rectally, for example in the form of suppositories or enemas, which include aqueous or oily solutions as well as suspensions and emulsions and foams. Such compositions are prepared following standard procedures, well known by those skilled in the art. For example, suppositories can be prepared by mixing the active ingredient with a conventional suppository base such as cocoa butter or other glycerides. In this case, the drug is mixed with a suitable non-irritating excipient which is solid at ordinary temperatures but liquid at the rectal temperature and will therefore melt in the rectum to release the drug. Such materials are cocoa butter and polyethylene glycols.

Generally, for compositions intended to be administered topically to the eye in the form of eye drops or eye ointments, the total amount of the compound of the present invention will be about 0.0001 to less than 4.0% (w/w).

Preferably, for topical ocular administration, the compositions administered according to the present invention will be formulated as solutions, suspensions, emulsions and other dosage forms.

The compositions administered according to the present invention may also include various other ingredients, including, but not limited to, tonicity agents, buffers, surfactants, stabilizing polymer, preservatives, co-solvents and viscosity building agents. Suitable pharmaceutical compositions of the present invention include a compound of the invention formulated with a tonicity agent and a buffer. The pharmaceutical compositions of the present invention may further optionally include a surfactant and/or a palliative agent and/or a stabilizing polymer.

Various tonicity agents may be employed to adjust the tonicity of the composition, preferably to that of natural tears for ophthalmic compositions. For example, sodium chloride, potassium chloride, magnesium chloride, calcium chloride, simple sugars such as dextrose, fructose, galactose, and/or simply polyols such as the sugar alcohols mannitol, sorbitol, xylitol, lactitol, isomaltitol, maltitol, and hydrogenated starch hydrolysates may be added to the composition to approximate physiological tonicity. Such an amount of tonicity agent will vary, depending on the particular agent to be added. In general, however, the compositions will have a tonicity agent in an amount sufficient to cause the final composition to have an ophthalmically acceptable osmolality (generally about 150-450 mOsm, preferably 250-350 mOsm and most preferably at approximately 290 mOsm). In general, the tonicity agents of the invention will be present in the range of 2 to 4% w/w. Preferred tonicity agents of the invention include the simple sugars or the sugar alcohols, such as D-mannitol.

An appropriate buffer system (e.g. sodium phosphate, sodium acetate, sodium citrate, sodium borate or boric acid) may be added to the compositions to prevent pH drift under storage conditions. The particular concentration will vary, depending on the agent employed. Preferably however, the buffer will be chosen to maintain a target pH within the range of pH 5 to 8, and more preferably to a target pH of pH 5 to 7.

Surfactants may optionally be employed to deliver higher concentrations of compound of the present invention. The surfactants function to solubilise the compound and stabilise colloid dispersion, such as micellar solution, microemulsion, emulsion and suspension. Examples of surfactants which may optionally be used include polysorbate, poloxamer, polyosyl 40 stearate, polyoxyl castor oil, tyloxapol, Triton, and sorbitan monolaurate. Preferred surfactants to be employed in the invention have a hydrophile/lipophile/balance “HLB” in the range of 12.4 to 13.2 and are acceptable for ophthalmic use, such as TritonX114 and tyloxapol.

Additional agents that may be added to the ophthalmic compositions of compounds of the present invention are demulcents which function as a stabilising polymer. The stabilizing polymer should be an ionic/charged example with precedence for topical ocular use, more specifically, a polymer that carries negative charge on its surface that can exhibit a zeta-potential of (−)10-50 mV for physical stability and capable of making a dispersion in water (i.e. water soluble). A preferred stabilising polymer of the invention would be polyelectrolyte, or polyelectrolytes if more than one, from the family of cross-linked polyacrylates, such as carbomers and Pemulen(R), specifically Carbomer 974p (polyacrylic acid), at 0.1-0.5% w/w.

Other compounds may also be added to the ophthalmic compositions of the compound of the present invention to increase the viscosity of the carrier. Examples of viscosity enhancing agents include, but are not limited to: polysaccharides, such as hyaluronic acid and its salts, chondroitin sulfate and its salts, dextrans, various polymers of the cellulose family; vinyl polymers; and acrylic acid polymers.

Topical ophthalmic products are typically packaged in multidose form. Preservatives are thus required to prevent microbial contamination during use. Suitable preservatives include: benzalkonium chloride, chlorobutanol, benzododecinium bromide, methyl paraben, propyl paraben, phenylethyl alcohol, edentate disodium, sorbic acid, polyquaternium-1, or other agents known to those skilled in the art. Such preservatives are typically employed at a level of from 0.001 to 1.0% w/v. Unit dose compositions of the present invention will be sterile, but typically unpreserved. Such compositions, therefore, generally will not contain preservatives.

Compositions suitable for buccal or sublingual administration include tablets, lozenges and pastilles where the compound of formula (I) is formulated with a carrier such as sugar and acacia, tragacanth, or gelatine and glycerine.

Compositions suitable for transdermal administration include ointments, gels and patches.

The composition may contain from 0.1% to 100% by weight, for example from 10 to 60% by weight, of the compound of formula (I), depending on the method of administration. The composition may contain from 0% to 99.9% by weight, such as 0% to 99% by weight, for example 40% to 90% by weight, of the carrier, depending on the method of administration. The composition may contain from 0.05 mg to 1000 mg, for example from 1.0 mg to 500 mg, such as from 1.0 mg to 50 mg, e.g. about 10 mg of the compound of formula (I), depending on the method of administration. The composition may contain from 50 mg to 1000 mg, for example from 100 mg to 400 mg of the carrier, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 1.0 to 500 mg, such as from 1.0 mg to 50 mg, e.g. about 10 mg and such unit doses may be administered more than once a day, for example two or three times a day. Such therapy may extend for a number of weeks or months.

In one embodiment of the invention, the compound of formula (I) is used in combination with a further therapeutic agent or agents. When the compound of formula (I) is used in combination with other therapeutic agents, the compounds may be administered either sequentially or simultaneously by any convenient route. Alternatively, the compounds may be administered separately.

Therapeutic agents which may be used in combination with the present invention include: corticosteroids (glucocorticoids), retinoids (e.g. acitretin, isotretinoin, tazarotene), anthralin, vitamin D analogues (e.g. cacitriol, calcipotriol), calcineurin inhibitors (e.g. tacrolimus, pimecrolimus), phototherapy or photochemotherapy (e.g. psoralen ultraviolet irradiation, PUVA) or other form of ultraviolet light irradiation therapy, ciclosporine, thiopurines (e.g. azathioprine, 6-mercaptopurine), methotrexate, anti-TNFα agents (e.g. infliximab, etanercept, adalimumab, certolizumab, golimumab and biosimilars), phosphodiesterase-4 (PDE4) inhibition (e.g. apremilast, crisaborole), anti-IL-17 agents (e.g. brodalumab, ixekizumab, secukinumab), anti-IL12/IL-23 agents (e.g. ustekinumab, briakinumab), anti-IL-23 agents (e.g. guselkumab, tildrakizumab), JAK (Janus Kinase) inhibitors (e.g. tofacitinib, ruxolitinib, baricitinib, filgotinib, upadacitinib), plasma exchange, intravenous immune globulin (IVIG), cyclophosphamide, anti-CD20 B cell depleting agents (e.g. rituximab, ocrelizumab, ofatumumab, obinutuzumab), anthracycline analogues (e.g. mitoxantrone), cladribine, sphingosine 1-phosphate receptor modulators or sphingosine analogues (e.g. fingolimod, siponimod, ozanimod, etrasimod), interferon beta preparations (including interferon beta 1b/1a), glatiramer, anti-CD3 therapy (e.g. OKT3), anti-CD52 targeting agents (e.g. alemtuzumab), leflunomide, teriflunomide, gold compounds, laquinimod, potassium channel blockers (e.g. dalfampridine/4-aminopyridine), mycophenolic acid, mycophenolate mofetil, purine analogues (e.g. pentostatin), mTOR (mechanistic target of rapamycin) pathway inhibitors (e.g. sirolimus, everolimus), antithymocyte globulin (ATG), IL-2 receptor (CD25) inhibitors (e.g. basiliximab, daclizumab), anti-IL-6 receptor or anti-IL-6 agents (e.g. tocilizumab, siltuximab), Bruton's tyrosine kinase (BTK) inhibitors (e.g. ibrutinib), tyrosine kinase inhibitors (e.g. imatinib), ursodeoxycholic acid, hydroxychloroquine, chloroquine, B cell activating factor (BAFF, also known as BLyS, B lymphocyte stimulator) inhibitors (e.g. belimumab, blisibimod), other B cell targeted therapy including fusion proteins targeting both APRIL (A PRoliferation-Inducing Ligand) and BLyS (e.g. atacicept), PI3K inhibitors including pan-inhibitors or those targeting the p110δ and/or p110γ containing isoforms (e.g. idelalisib, copanlisib, duvelisib), interferon α receptor inhibitors (e.g. anifrolumab, sifalimumab), T cell co-stimulation blockers (e.g. abatacept, belatacept), thalidomide and its derivatives (e.g. lenalidomide), dapsone, clofazimine, leukotriene antagonists (e.g. montelukast), theophylline, anti-IgE therapy (e.g. omalizumab), anti-IL-5 agents (e.g. mepolizumab, reslizumab), long-acting muscarinic agents (e.g. tiotropium, aclidinium, umeclidinium), PDE4 inhibitors (e.g. roflumilast), riluzole, free radical scavengers (e.g. edaravone), proteasome inhibitors (e.g. bortezomib), complement cascade inhibitors including those directed against C5 (e.g. eculizumab), immunoadsor, antithymocyte globulin, 5-aminosalicylates and their derivatives (e.g. sulfasalazine, balsalazide, mesalamine), anti-integrin agents including those targeting α4β1 and/or α4β7 integrins (e.g. natalizumab, vedolizumab), anti-CD11-α agents (e.g. efalizumab), non-steroidal anti-inflammatory drugs (NSAIDs) including the salicylates (e.g. aspirin), propionic acids (e.g. ibuprofen, naproxen), acetic acids (e.g. indomethacin, diclofenac, etodolac), oxicams (e.g. meloxicam) and fenamates (e.g. mefenamic acid), selective or relatively selective COX-2 inhibitors (e.g. celecoxib, etroxicoxib, valdecoxib and etodolac, meloxicam, nabumetone), colchicine, IL-4 receptor inhibitors (e.g. dupilumab), topical/contact immunotherapy (e.g. diphenylcyclopropenone, squaric acid dibutyl ester), anti-IL-1 receptor therapy (e.g. anakinra), IL-1β inhibitor (e.g. canakinumab), IL-1 neutralising therapy (e.g. rilonacept), chlorambucil, specific antibiotics with immunomodulatory properties and/or ability to modulate NRF2 (e.g. tetracyclines including minocycline, clindamycin, macrolide antibiotics), anti-androgenic therapy (e.g. cyproterone, spironolactone, finasteride), pentoxifylline, ursodeoxycholic acid, obeticholic acid, fibrate, cystic fibrosis transmembrane conductance regulator (CFTR) modulators, VEGF (vascular endothelial growth factor) inhibitors (e.g. bevacizumab, ranibizumab, pegaptanib, aflibercept), pirfenidone, and mizoribine.

Compounds of formula (I) may display one or more of the following desirable properties:
low IC₅₀ values for inhibiting release of cytokines e.g. IL-1β and/or IL-6, from cells;
low EC₅₀ and/or high Eₘₐₓ values for activating the NRF2 pathway;
enhanced efficacy through improved hydrolytic stability of carboxylic acid esters and/or augmented maximum response;
reduced dose and dosing frequency through improved pharmacokinetics;
improved oral systemic bioavailability;
reduced plasma clearance following intravenous dosing;
improved metabolic stability e.g. as demonstrated by improved stability in plasma and/or hepatocytes;
augmented cell permeability;
enhanced aqueous solubility;
good tolerability, for example, by limiting the flushing and/or gastrointestinal side effects provoked by oral DMF (Hunt T. et al., 2015; WO2014/152494A1, incorporated herein by reference), possibly by reducing or eliminating HCA2 activity;
low toxicity at the relevant therapeutic dose;
distinct anti-inflammatory profiles resulting from varied electrophilicities, leading to differential targeting of the cysteine proteome (van der Reest J. et al., 2018) and, therefore, modified effects on gene activation;
glutathione-sparing actions;
avoiding the oncometabolite fumaric acid (Kulkarni R. A. et al., 2019).

### ABBREVIATIONS

Ac acetyl
aq. aqueous
BBFO broadband fluorine observe
BEH ethylene bridged hybrid
n-Bu n-butyl
CSH charged surface hybrid
d doublet
DAD diode array detector
DBU 1,8-diazabicyclo(5.4.0)undec-7-ene
DCC N,N′-dicyclohexylcarbodiimide
DCM dichloromethane
DIPEA N,N-diisopropylethylamine
DMAP 4-dimethylaminopyridine
DMSO dimethyl sulfoxide
EDC 1-ethyl-3-(3-dimethylaminopropyl)carbodiimide
Et ethyl
ES⁺ electrospray
FBS fetal bovine serum
g gram(s)
GSH glutathione
h hour(s)
HPLC high-performance liquid chromatography
IL interleukin
IPA isopropyl alcohol
LCMS liquid chromatography-mass spectrometry
m multiplet
M molar concentration/molar mass
m/z mass to charge ratio
(M)Hz (mega)hertz
min(s) minute(s)
mL millilitres
mmol millimole
MS mass spectrometry
Ms methanesulfonyl
MTBE methyl tertiary-butyl ether
NMP N-methyl-2-pyrrolidone
NMR nuclear magnetic resonance
PBS phosphate buffered saline
PDA photodiode array
4OI 4-octyl itaconic acid
rpm revolutions per minute
RT room temperature
s singlet
SFC supercritical fluid chromatography
t triplet
TBS tert-butyldimethylsilyl
TFA trifluoroacetic acid
THF tetrahydrofuran
Ts toluenesulfonyl
μL microlitre
μM micromolar
UPLC ultra performance liquid chromatography
wt. weight
° C. degrees centigrade

### EXAMPLES

Analytical Equipment

NMR spectra were recorded using a Bruker 400 MHz Avance Ill spectrometer fitted with a BBFO 5 mm probe, or a Bruker 500 MHz Avance III HD spectrometer equipped with a Bruker 5 mm SmartProbe™. Spectra were measured at 298 K, unless indicated otherwise, and were referenced relative to the solvent resonance. The chemical shifts are reported in parts per million. Data were acquired using Bruker TopSpin software.

UPLC/MS analysis was carried out on a Waters Acquity UPL system using either a Waters Acquity CSH 018 or BEH 018 column (2.1×30 mm) maintained at a temperature of 40° C. and eluted with a linear acetonitrile gradient appropriate for the lipophilicity of the compound over 3 or 10 minutes at a constant flow rate of 0.77 mL/min. The aqueous portion of the mobile phase was either 0.1% Formic Acid (CSH 018 column) or 10 mM Ammonium Bicarbonate (BEH 018 column). LC-UV chromatograms were recorded using a Waters Acquity PDA detector between 210 and 400 nm. Mass spectra were recorded using a Waters Acquity Qda detector with electrospray ionisation switching between positive and negative ion mode. Sample concentration was adjusted to give adequate UV response.

The following analytical LCMS equipment and methods were used:

LCMS/HPLC Instrument Details

| System | Instrument Name | LC Detector | ELS detector | Mass detector |
|---|---|---|---|---|
| 2 | Agilent LCMS 1200 | G1315C DAD | 380 ELSD | Agilent G6110A |

LCMS/HPLC Method Details

| | | | | | | | Flow |
|---|---|---|---|---|---|---|---|
| Method | Solvent | | | UV | Mass | Column | Rate |
| Name | System | Column | Gradient | range | Range | Temp. ° C. | ml/min |
| B | A) water + | Waters X-Bridge C18 | From 95:5 to | 190-400 nm | 100-1100 amu | 40 | 2.0 |
| | 0.05% TFA | (50 mm × 4.6 mm × | 0:100 in 1.6 min, | | | | |
| | B) acetonitrile + | 3.5 μm) | 0:100 for 1.4 min, | | | | |
| | 0.05% TFA | | from 0:100 to | | | | |
| | | | 95:5 in 0.05 min, | | | | |
| | | | 95:5 for 0.7 min | | | | |
| C | A) water + | Halo C18 (30 mm × | From 95:5 to | 190-400 nm | 100-1100 amu | 40 | 3.0 |
| | 0.05% TFA | 4.6 mm × 2.7 μm) | 0:100 in 0.8 min, | | | | |
| | B) acetonitrile + | | 0:100 for 0.4 min, | | | | |
| | 0.05% TFA | | from 0:100 to | | | | |
| | | | 95:5 in 0.01 min, | | | | |
| | | | 95:5 for 0.2 min | | | | |

Commercial Materials

All starting materials are commercially available unless otherwise described herein. Dimethyl itaconate was purchased from Sigma-Aldrich (product number: 109533); 4-octyl itaconate was purchased from BOC biosciences (product number: B0001-007866).

General Methods

Unless otherwise stated all reactions were stirred. Organic solutions were routinely dried over anhydrous magnesium sulfate. Hydrogenations were performed on a Thales H-cube flow reactor under the conditions stated or under pressure in a gas autoclave (bomb).

Preparation of Intermediates

### Preparation of 1-(4-(trifluoromethyl) phenyl)cyclobutan-1-ol

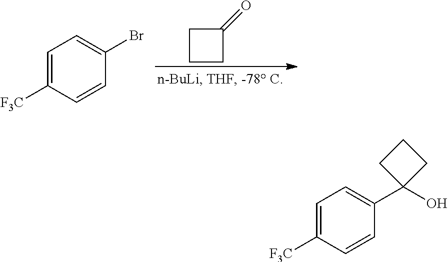 [SMILES: FC(F)(F)c1ccc(Br)cc1 ; O=C1CCC1 ; OC1(c2ccc(C(F)(F)F)cc2)CCC1]

To a solution of 1-bromo-4-(trifluoromethyl)benzene (6.0 g, 26.9 mmol) in THF (50 mL) at −78° C. was added a solution of n-BuLi in THF (2.5 M, 11.8 mL, 29.5 mmol) and the mixture was stirred at −78° C. for 1 h. Then cyclobutanone (2.06 g, 29.5 mmol) was added and the mixture was stirred at −78° C. for 5 h. The reaction mixture was quenched with saturated aqueous NH₄Cl solution (30 mL) and the phases were separated. The aqueous layer was extracted with MTBE (2×20 mL) and the combined organic layers were washed with brine, dried over Na₂SO₄, filtered and concentrated under reduced pressure at 30° C. The residue was purified by flash column chromatography (80 g silica, 0-14% MTBE/petroleum ether) to give 1-(4-(trifluoromethyl)phenyl)cyclobutanol (2.6 g, 12.0 mmol, 45%) as a yellow oil. ¹H NMR (400 MHz, CDCl₃) δ: 7.62 (s, 4H), 2.58-2.51 (m, 2H), 2.43-2.35 (m, 2H), 2.12-2.01 (m, 1H), 1.81-1.68 (m, 1H). One exchangeable proton not observed.

The following compounds were prepared by an analogous method:
1-(4-bromophenyl)cyclobutan-1-ol from 1,4-dibromobenzene. ¹H NMR (400 MHz, CDCl₃) δ: 7.53-7.44 (m, 2H), 7.42-7.33 (m, 2H), 2.55-2.45 (m, 2H), 2.39-2.29 (m, 2H), 2.08-1.95 (m, 1H), 1.75-1.61 (m, 1H). One exchangeable proton not observed.
1-(4-chlorophenyl)cyclobutan-1-ol from 1-bromo-4-chlorobenzene. ¹H NMR (400 MHz, CDCl₃) δ: 7.45-7.38 (m, 2H), 7.36-7.29 (d, J=4.2 Hz, 2H), 2.56-2.45 (m, 2H), 2.41-2.29 (m, 2H), 2.08-1.95 (m, 1H), 1.75-1.60 (m, 1H). One exchangeable proton not observed.
1-(2,5-dichlorophenyl)cyclobutan-1-ol from 1-bromo-2,5-dichlorobenzene. ¹H NMR (400 MHz, CDCl₃) δ: 7.34 (d, J=2.5 Hz, 1H), 7.29 (d, J=8.4 Hz, 1H), 7.19 (dd, J=8.5, 2.5 Hz, 1H), 2.70-2.56 (m, 2H), 2.49-2.37 (m, 2H), 2.26-2.13 (m, 1H), 1.79-1.67 (m, 1H). One exchangeable proton not observed.
1-(2-chloro-4-(trifluoromethoxy)phenyl)cyclobutan-1-ol from 1-bromo-2-chloro-4-(trifluoromethoxy)benzene. ¹H NMR (400 MHz, CDCl₃) δ: 7.32-7.27 (m, 1H), 7.22 (t, J=8.0 Hz, 1H), 7.18-7.13 (m, 1H), 2.80-2.68 (m, 2H), 2.62-2.53 (m, 2H), 2.41-2.25 (m, 1H), 1.90-1.76 (m, 1H). One exchangeable proton not observed.
1-(2,3-dichlorophenyl)cyclobutan-1-ol from 1-bromo-2,3-dichlorobenzene. ¹H NMR (400 MHz, CDCl₃) δ: 7.40 (dd, J=7.9, 1.6 Hz, 1H), 7.29 (dd, J=7.8, 1.6 Hz, 1H), 7.20 (t, J=7.8 Hz, 1H), 2.71-2.59 (m, 2H), 2.52-2.41 (m, 2H), 2.28-2.15 (m, 1H), 1.79-1.67 (m, 1H). One exchangeable proton not observed.
1-(2,6-dichlorophenyl)cyclobutan-1-ol from 1-bromo-2,6-dichlorobenzene. ¹H NMR (400 MHz, CDCl₃) δ: 7.26 (d, J=8.0 Hz, 2H), 7.09 (t, J=7.9 Hz, 1H), 2.87-2.75 (m, 2H), 2.72-2.62 (m, 2H), 2.36-2.22 (m, 1H), 1.84-1.73 (m, 1H). One exchangeable proton not observed.
1-(4-(trifluoromethoxy)phenyl)cyclobutan-1-ol from 1-bromo-4-(trifluoromethoxy)benzene. 1H NMR (400 MHz, CDCl₃) δ: 7.56-7.48 (m, 2H), 7.24-7.17 (m, 2H), 2.60-2.47 (m, 2H), 2.43-2.30 (m, 2H), 2.10-1.94 (m, 1H), 1.78-1.63 (m, 1H). One exchangeable proton not observed.
1-(2,4-dichlorophenyl)cyclobutan-1-ol from 2,4-dichloro-1-iodobenzene. ¹H NMR (400 MHz, DMSO-d₆) δ: 7.52-7.47 (m, 1H), 7.44-7.33 (m, 2H), 5.46 (s, 1H), 2.60-2.47 (m, 2H), 2.35-2.23 (m, 2H), 2.10-1.96 (m, 1H), 1.64-1.51 (m, 1H).
1-(2-chloro-4-(trifluoromethyl)phenyl)cyclobutan-1-ol from 1-bromo-2-chloro-4-(trifluoromethyl)benzene. ¹H NMR (400 MHz, CDCl3) δ: 7.63 (s, 1H), 7.55-7.47 (m, 2H), 2.72-2.61 (m, 2H), 2.53-2.41 (m, 2H), 2.31-2.16 (m, 1H), 1.81-1.69 (m, 1H). One exchangeable proton not observed.
1-(4-bromo-2-chlorophenyl)cyclobutan-1-ol from 4-bromo-2-chloro-1-iodobenzene. 1H NMR (400 MHz, DMSO-d₆) δ: 7.63 (d, J=2.1 Hz, 1H), 7.51 (dd, J=8.3, 2.0 Hz, 1H), 7.35 (d, J=8.3 Hz, 1H), 5.47 (s, 1H), 2.59-2.50 (m, 2H), 2.34-2.23 (m, 2H), 2.09-1.95 (m, 1H), 1.64-1.51 (m, 1H).
1-(3-chloro-4-methylphenyl)cyclobutan-1-ol from 4-bromo-2-chloro-1-methylbenzene. ¹H NMR (400 MHz, CDCl₃) δ: 7.48 (d, J=1.9 Hz, 1H), 7.29 (dd, J=7.9, 1.9 Hz, 1H), 7.22 (d, J=7.9 Hz, 1H), 2.57-2.47 (m, 2H), 2.42-2.30 (m, 2H), 2.30 (s, 3H), 2.08-1.95 (m, 1H), 1.76-1.62 (m, 1H). One exchangeable proton not observed.
1-(4-(trifluoromethyl)phenyl)cyclopentan-1-ol from 1-bromo-4-(trifluoromethyl)benzene and cyclopentanone. ¹H NMR (400 MHz, CDCl₃) δ: 7.64-7.56 (m, 4H), 2.07-1.93 (m, 6H), 1.92-1.82 (m, 2H). One exchangeable proton not observed.
1-(4′-fluoro-[1,1′-biphenyl]-4-yl)cyclobutan-1-ol from 4-bromo-4′-fluoro-1,1′-biphenyl. ¹H NMR (400 MHz, CDCl₃) δ: 7.62-7.50 (m, 6H), 7.18-7.08 (m, 2H), 2.67-2.55 (m, 2H), 2.47-2.35 (m, 2H), 2.12-1.98 (m, 2H), 1.80-1.66 (m, 1H).
1-((4-(trifluoromethyl)phenyl)ethynyl)cyclobutan-1-ol from 1-ethynyl-4-(trifluoromethyl)benzene. LCMS: (System 2, Method C) m/z 223.2 (M−OH)⁺ (ES⁺).
3-(4-(trifluoromethyl)phenyl)oxetan-3-ol from 1-bromo-4-(trifluoromethyl)benzene and oxetan-3-one. ¹H NMR (400 MHz, CDCl₃) δ: 7.78 (d, J=8.4 Hz, 2H), 7.69 (d, J=8.4 Hz, 2H), 4.95 (d, J=7.6 Hz, 2H), 4.89 (d, J=7.6 Hz, 2H). One exchangeable proton not observed.
3,3-difluoro-1-(4-(trifluoromethyl)phenyl)cyclobutan-1-ol from 1-iodo-4-(trifluoromethyl)benzene and 3,3-difluorocyclobutanone. ¹H NMR (400 MHz, CDCl₃) δ7.67-7.63 (m, 2H), 7.62-7.25 (m, 2H), 3.20-3.01 (m, 4H). One exchangeable proton not observed.

### Preparation of 2,2,2-trichloroethyl 3-hydroxypropanoate

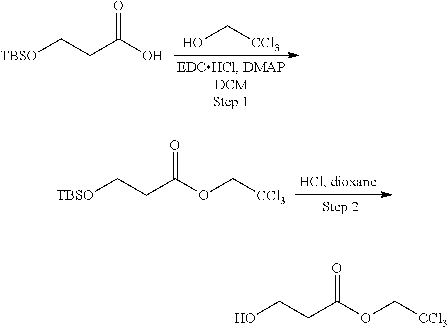

Step 1

To a solution of 3-((tert-butyldimethylsilyl)oxy)propanoic acid (1.9 g, 9.3 mmol), 2,2,2-trichloroethan-1-ol (1.27 g, 8.5 mmol) and DMAP (1.56 g, 12.8 mmol) in DCM (47 mL) at 0° C. was added EDC·HCl (2.45 g, 12.8 mmol) and the resulting pale yellow mixture was stirred at room temperature for 16 h. The mixture was quenched with saturated aqueous NH₄Cl solution (30 mL), the phases were separated, and the aqueous phase was extracted with EtOAc (2×40 mL). The separated organic phase was washed with brine, dried over Na₂SO₄ and filtered. The filtrate was concentrated under reduced pressure at 35° C., and the residue was purified by flash column chromatography (40 g silica, 0-9% MTBE/petroleum ether) to give 2,2,2-trichloroethyl 3-((tert-butyldimethylsilyl)oxy)propanoate (800 mg, 2.38 mmol, 25%) as a colorless oil. ¹H NMR (400 MHz, CDCl₃) δ: 4.75 (s, 2H), 3.95 (t, J=6.2 Hz, 2H), 2.68 (t, J=6.3 Hz, 2H), 0.87 (s, 9H), 0.06 (s, 6H).

Step 2

A solution of 2,2,2-trichloroethyl 3-((tert-butyldimethylsilyl)oxy)propanoate (800 mg, 2.38 mmol) in HCl solution in 1,4-dioxane (4 M, 10 mL) was stirred at room temperature for 16 h. The mixture was concentrated under reduced pressure at 30° C. and the residue was purified by flash column chromatography (40 g silica, 0-10% MTBE/petroleum ether) to give 2,2,2-trichloroethyl 3-hydroxypropanoate (400 mg, 1.81 mmol, 75%) as a pale yellow oil. LCMS: (System 2, Method C) m/z 221.2/223.2 (M+H)⁺ (ES⁺).

### Preparation of 4-methoxybenzyl (S)-4,4,4-trifluoro-3-hydroxybutanoate and 4-methoxybenzyl (R)-4,4,4-trifluoro-3-hydroxybutanoate

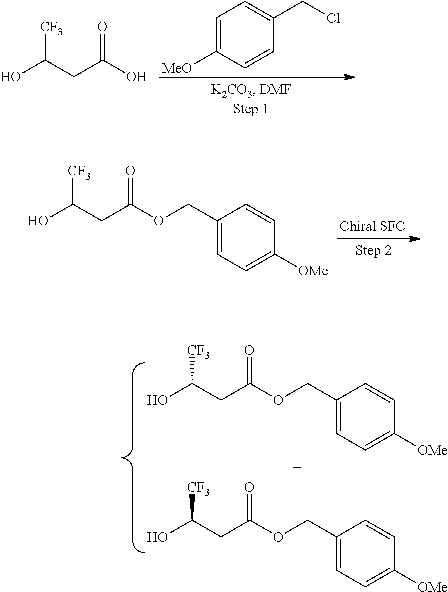

Step 1

To a mixture of 4,4,4-trifluoro-3-hydroxybutanoic acid (45 g, 285 mmol) and K₂CO₃ (39.3 g, 285 mmol) in DMF (1200 mL) was added PMBCl (44.5 g, 285 mmol) at 0° C., and the reaction mixture was stirred at room temperature overnight. The reaction mixture was quenched with water and extracted with EtOAc. The organic layers were washed with water and brine, dried over anhydrous Na₂SO₄ and filtered. The filtrate was concentrated under reduced pressure at 40° C. and the residue was purified by flash column chromatography (120 g SiO₂, 0-40% MTBE/petroleum ether) to give 4-methoxybenzyl 4,4,4-trifluoro-3-hydroxybutanoate (55 g, 198 mmol, 70%) as a white solid. LCMS (System 2, Method B) m/z 301.2 (M+Na)⁺ (ES⁺).

Step 2

4-Methoxybenzyl 4,4,4-trifluoro-3-hydroxybutanoate (55 g, 198 mmol) was resolved into separate enantiomers using chiral SFC (Column: CHIRALPAK AD-5 5 μm 30×250 mm; Column temperature: 35° C.; Flow rate: 45 mL/min; Solvent system: 20% IPA/CO₂; Collection wavelength: 215 nm). The collected fractions were concentrated under reduced pressure at 40° C. to give 4-methoxybenzyl (S)-4,4,4-trifluoro-3-hydroxybutanoate (21 g, 75.5 mmol, 38%) as the first eluting peak and 4-methoxybenzyl (R)-4,4,4-trifluoro-3-hydroxybutanoate (21 g, 75.5 mmol, 38%) as the second eluting peak. Both compounds were isolated as white solids. Chiral SFC analysis (Column: CHIRALPAKAD-3 3 μm 4.6×100 mm; Column temperature: 35° C.; Flow rate: 2 mL/min; Solvent system: 20% (0.2% (7M NH₃/MeOH) in MeOH)/CO₂; Collection wavelength: 215 nm): 4-methoxybenzyl (S)-4,4,4-trifluoro-3-hydroxybutanoate Rt=0.943 min, 99.1% ee; 4-methoxybenzyl (R)-4,4,4-trifluoro-3-hydroxybutanoate Rt=1.281 min, 99.4% ee.

### Preparation of 2,2,2-trichloroethyl (S)-4,4,4-trifluoro-3-hydroxybutanoate

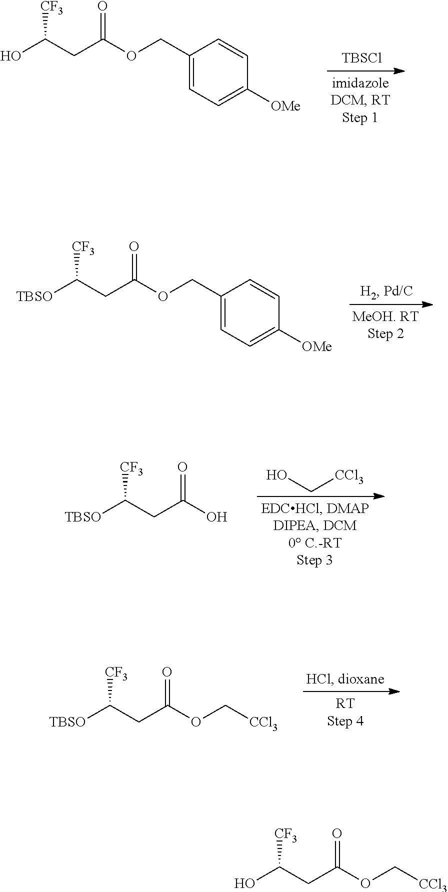

Step 1

A mixture of 4-methoxybenzyl (S)-4,4,4-trifluoro-3-hydroxybutanoate (4.00 g, 14.4 mmol), tert-butylchlorodimethylsilane (5.43 g, 36.0 mmol) and imidazole (2.94 g, 43.2 mmol) in DCM (60 mL) was stirred at room temperature overnight. The mixture was quenched with H₂O (50 mL), the phases were separated and the aqueous phase was extracted with DCM (2×500 mL). The combined organic phases were washed with brine, dried over Na₂SO₄ and filtered. The filtrate was concentrated under reduced pressure at 25° C., and the residue was purified by flash column chromatography (40 g silica, 0-10% MTBE/petroleum ether) to give 4-methoxybenzyl (S)-3-((tert-butyldimethylsilyl)oxy)-4,4,4-trifluorobutanoate (5.1 g, 13.0 mmol, 91%) as a pale yellow oil. LCMS (System 2, Method C) m/z 415.0 (M+Na)⁺ (ES⁺).

Step 2

To a solution of (S)-3-((tert-butyldimethylsilyl)oxy)-4,4,4-trifluorobutanoate (5.1 g, 13.0 mmol) in MeOH (60 mL) was added 10% palladium on carbon (1.02 g) and the mixture was stirred at room temperature under an atmosphere of hydrogen for 3 h. The mixture was filtered through Celite and the filtrate was concentrated under reduced pressure at 35° C. to give crude (S)-3-((tert-butyldimethylsilyl)oxy)-4,4,4-trifluorobutanoic acid (3.9 g, 14.3 mmol, 100%) as a pale yellow oil, which was used directly in the next step. ¹H NMR (400 MHz, CDCl₃) δ: 4.54-4.43 (m, 1H), 2.80-2.61 (m, 2H), 0.87 (s, 9H), 0.13 (s, 3H), 0.09 (s, 3H). One exchangeable proton not observed.

Step 3

A mixture of (S)-3-((tert-butyldimethylsilyl)oxy)-4,4,4-trifluorobutanoic acid (3.9 g, 14.3 mmol), 2,2,2-trichloroethanol (4.27 g, 28.7 mmol), DMAP (2.63 g, 21.5 mmol), EDC·HCl (5.51 g, 28.7 mmol) and DIPEA (5.55 g, 43.0 mmol) in DCM (70 mL) at 0° C. was stirred overnight and allowed to warm to room temperature. The mixture was quenched with dilute aqueous HCl (0.5 M, 50 mL), the phases were separated, and the aqueous layer was extracted with DCM (2×50 mL). The combined organic phases were washed with brine, dried over Na₂SO₄ and filtered. The filtrate was concentrated under reduced pressure at 35° C., and the residue was purified by flash column chromatography (80 g silica, 0-9% MTBE/petroleum ether) to give 2,2,2-trichloroethyl (S)-3-((tert-butyldimethylsilyl)oxy)-4,4,4-trifluorobutanoate (2.9 g, 7.20 mmol, 56%) as a yellow oil. ¹H NMR (400 MHz, CDCl₃) δ: 4.81-4.72 (m, 2H), 4.59-4.49 (m, 1H), 2.90-2.75 (m, 2H), 0.91-0.81 (m, 9H), 0.14 (s, 3H), 0.10 (s, 3H).

Step 4

A solution of 2,2,2-trichloroethyl (S)-3-((tert-butyldimethylsilyl)oxy)-4,4,4-trifluorobutanoate (2.9 g, 7.20 mmol) in HCl solution in 1,4-dioxane (4 M, 60 mL) was stirred at room temperature for 2 days. The mixture was concentrated under reduced pressure at 35° C. and the residue was purified by flash column chromatography (0-10% EtOAc/petroleum ether) to give 2,2,2-trichloroethyl (S)-4,4,4-trifluoro-3-hydroxybutanoate (340 mg, 1.17 mmol, 16%) as a yellow oil. ¹H NMR (400 MHz, CDCl₃) δ: 4.88-4.74 (m, 2H), 4.59-4.47 (m, 1H), 3.01 (d, J=5.6 Hz, 1H), 2.96-2.81 (m, 2H).

### Preparation of 1-(3-chloro-4-(trifluoromethyl) phenyl)cyclobutan-1-ol

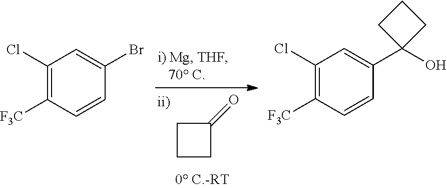 [SMILES: FC(F)(F)c1ccc(Br)cc1Cl ; O=C1CCC1 ; OC1(c2ccc(C(F)(F)F)c(Cl)c2)CCC1]

To a suspension of Mg metal (467 mg, 19.6 mmol) in THF (50 mL) was added 4-bromo-2-chloro-1-(trifluoromethyl)benzene (5 g, 19.6 mmol) at 70° C. and the mixture was stirred at 70° C. for 1 h. The solution was cooled to 0° C., cyclobutanone (1.37 g, 19.6 mmol) was added, and the mixture was stirred at room temperature for 3 h. The reaction mixture was quenched with saturated aqueous ammonium chloride (50 mL) and the phases were separated. The aqueous layer was extracted with MTBE (2×50 mL), the combined organic layers were washed with brine, dried over Na₂SO₄, filtered and concentrated under reduced pressure at 30° C. The residue was purified by flash column chromatography (80 g silica, 0-14% MTBE/petroleum ether) to give 1-(3-chloro-4-(trifluoromethyl)phenyl)cyclobutanol (2.3 g, 9.18 mmol, 47%) as a yellow oil. ¹H NMR (400 MHz, CDCl₃) δ: 7.71-7.63 (m, 2H), 7.49 (d, J=8.0 Hz, 1H), 2.59-2.47 (m, 2H), 2.45-2.33 (m, 2H), 2.17-2.01 (m, 1H), 1.86-1.71 (m, 1H). One exchangeable proton not observed.

The following compounds were prepared by an analogous method:
1-(3,5-dichloro-4-fluorophenyl)cyclobutan-1-ol from 5-bromo-1,3-dichloro-2-fluorobenzene. ¹H NMR (400 MHz, CDCl₃) δ: 7.44 (d, J=6.3 Hz, 2H), 2.55-2.44 (m, 2H), 2.41-2.30 (m, 2H), 2.13-1.99 (m, 1H), 1.81-1.67 (m, 1H). One exchangeable proton not observed.
1-(3,5-dichloro-4-methoxyphenyl)cyclobutan-1-ol from 5-bromo-1,3-dichloro-2-methoxybenzene. ¹H NMR (400 MHz, CDCl₃) δ: 7.41 (s, 2H), 3.89 (s, 3H), 2.54-2.43 (m, 2H), 2.40-2.28 (m, 2H), 2.10-1.96 (m, 1H), 1.78-1.64 (m, 1H). One exchangeable proton not observed.
1-(3,5-difluoro-4-(trifluoromethyl)phenyl)cyclobutan-1-ol from 5-bromo-1,3-difluoro-2-(trifluoromethyl)benzene. ¹H NMR (400 MHz, CDCl₃) δ: 7.16 (d, J=11.1 Hz, 2H), 2.55-2.44 (m, 2H), 2.44-2.34 (m, 2H), 2.17-2.03 (m, 1H), 1.88-1.74 (m, 1H). One exchangeable proton not observed.
1-(4-chlorobenzyl)cyclobutan-1-ol from 1-(bromomethyl)-4-chlorobenzene. ¹H NMR (400 MHz, CDCl₃) δ: 7.30-7.26 (m, 2H), 7.22-7.17 (m, 2H), 2.86 (s, 2H), 2.18-2.08 (m, 2H), 2.05-1.93 (m, 2H), 1.88-1.82 (m, 2H). One exchangeable proton not observed.

### Preparation of 2,2,2-trichloroethyl (R)-4,4,4-trifluoro-3-hydroxybutanoate

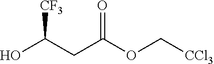

Prepared by an analogous method to 2,2,2-trichloroethyl (S)-4,4,4-trifluoro-3-hydroxybutanoate starting from 4-methoxybenzyl (R)-4,4,4-trifluoro-3-hydroxybutanoate (4.00 g, 14.4 mmol). Yield 600 mg. Yellow oil. ¹H NMR (400 MHz, CDCl₃) δ: 4.89-4.73 (m, 2H), 4.60-4.47 (m, 1H), 3.08 (d, J=5.6 Hz, 1H), 2.96-2.80 (m, 2H).

### Preparation of 1-((4-(trifluoromethyl) phenyl)ethynyl)cyclobutyl 2-bromoacetate

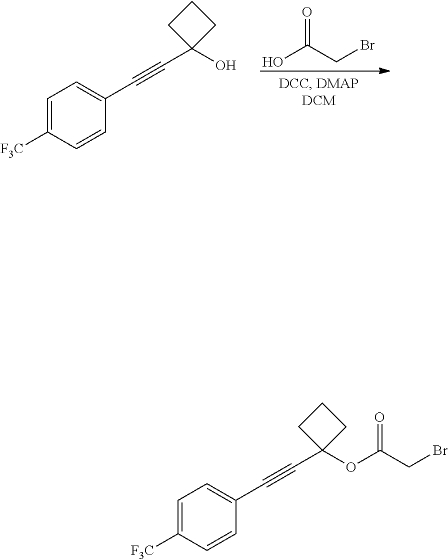 [SMILES: OC1(C#Cc2ccc(C(F)(F)F)cc2)CCC1 ; O=C(O)CBr ; O=C(CBr)OC1(C#Cc2ccc(C(F)(F)F)cc2)CCC1]

To a solution of 2-bromoacetic acid (4.34 g, 31.2 mmol) in DCM (90 mL) at room temperature was added DCC (6.43 g, 31.2 mmol), 1-((4-(trifluoromethyl)phenyl)ethynyl)cyclobutan-1-ol (5.00 g, 20.8 mmol) and DMAP (10.98 g, 72.2 mmol), and the mixture was stirred at room temperature for 4 h. The reaction mixture was filtered, and the filtrate was diluted with water (100 mL) and MTBE (100 mL). The phases were separated, and the aqueous layer was extracted with DCM (2×100 mL). The combined organic layers were washed with brine, dried over Na₂SO₄, filtered and concentrated under reduced pressure at 30° C. The residue was purified by flash column chromatography (80 g silica, 0-20% MTBE/petroleum ether) to give 1-((4-(trifluoromethyl)phenyl)ethynyl)cyclobutyl 2-bromoacetate (5.90 g, 78%) as a yellow oil. ¹H NMR (400 MHz, CDCl₃) δ: 7.76 (d, J=8.1 Hz, 2H), 7.66 (d, J=8.0 Hz, 2H), 4.20 (s, 2H), 2.69-2.56 (m, 2H), 2.56-2.44 (m, 2H), 2.05-1.87 (m, 2H).

### Preparation of 1-(4-(trifluoromethyl)phenyl)cyclopropanol

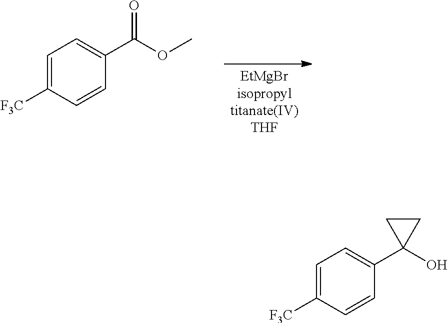

To the solution of methyl 4-(trifluoromethyl)benzoate (4 g, 19.6 mmol) and titanium tetraisopropoxide (4.14 g, 30 mmol) in THF (50 mL) was added ethyl magnesium bromide (20 mL, 60 mmol, 3M in ether) slowly at 0° C.; and the mixture was stirred at room temperature overnight. The reaction mixture was quenched with water (30 mL) and stirred for 1 h. The gray precipitate was filtered off and the filtrate was extracted with MTBE (3×40 mL). The combined organic layers were washed by brine, dried over Na₂SO₄, filtered and concentrated under reduced pressure. The residue was purified by column chromatography (80 g silica, 0-10% MTBE/petroleum ether) to give 1-(4-(trifluoromethyl)phenyl)cyclopropanol (1 g, 24%) as colorless oil. ¹H NMR (400 MHz, CDCl3) δ: 7.45 (d, J=8.4 Hz, 2H), 7.37 (d, J=8.4 Hz, 2H), 3.40 (s, 1H), 1.37-1.34 (m, 2H), 1.12-1.09 (m, 2H).

### Preparation of (9H-fluoren-9-yl)methyl 2-(3-hydroxyoxetan-3-yl)acetate

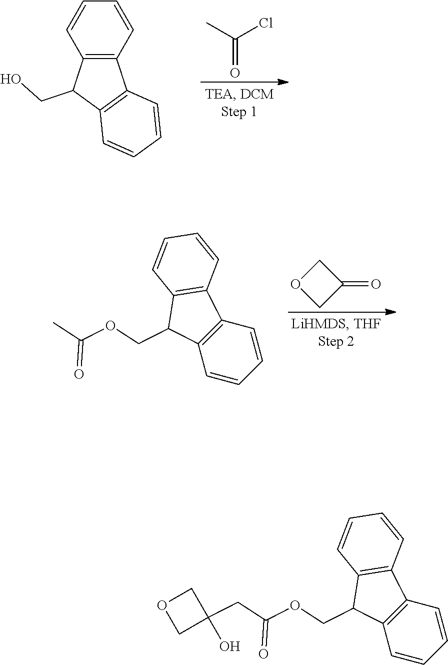

Step 1

To the solution of (9H-fluoren-9-yl)methanol (1.96 g, 10.0 mmol) and triethylamine (1.32 g, 13.0 mmol) in DCM (20 mL) was added acetyl chloride (941 mg, 12.0 mmol) in portions at 0° C., and the mixture was stirred at room temperature for 1 h. The mixture was quenched with 1N HCl (10 mL) and the aqueous layer was extracted with DCM (2×10 mL). The combined organic layers were washed by brine, dried over Na₂SO₄ and concentrated under reduced pressure. The residue was purified by flash column chromatography (40 g silica, 0-15% MTBE/petroleum ether) to give (9H-fluoren-9-yl)methyl acetate (2.35 g, 99% yield) as light-yellow oil ¹H NMR (400 MHz, CDCl₃) δ: 7.76 (d, J=7.6 Hz, 2H), 7.59 (d, J=7.2 Hz, 2H), 7.40 (t, J=7.6 Hz, 2H), 7.33-7.29 (m, 2H), 4.36 (d, J=7.2 Hz, 2H), 4.21 (t, J=7.2 Hz, 1H), 2.14 (s, 3H).

Step 2

To the solution of (9H-fluoren-9-yl)methyl acetate (500 mg, 2.10 mmol) in THF (10 mL) was added LiHMDS (2.3 mL, 2.30 mmol, 1M in THF) at −70° C.; and the mixture was stirred at −70° C. for 1 h. Oxetan-3-one (152 mg, 2.10 mmol) was added at −70° C., and the mixture was stirred at −70° C. for 1 h. The mixture was quenched with saturated NH₄Cl (10 mL) and the aqueous layer was extracted with MTBE (3×10 mL). The combined organic layers were washed by brine, dried over Na₂SO₄ concentrated under reduced pressure. The residue was purified by flash column chromatography (20 g silica, 0-20% MTBE/petroleum ether) to give (9H-fluoren-9-yl)methyl 2-(3-hydroxyoxetan-3-yl)acetate (260 mg, 40% yield) as white solid. LCMS: (System 2, Method C) m/z 333.0 (M+Na)⁺ (ES⁺).

### Preparation of 2-methylene-4-oxo-4-(1-(4-(trifluoromethyl)phenyl)cyclobutoxy)butanoic acid

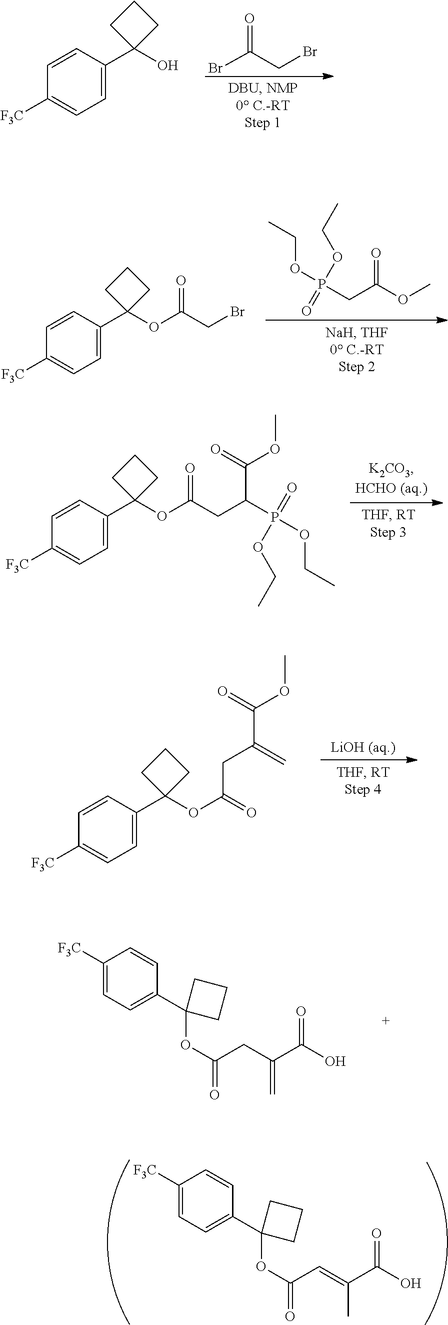

Step 1

To a solution of 1-(4-(trifluoromethyl)phenyl)cyclobutan-1-ol (16.5 g, 76.3 mmol) and DBU (23.2 g, 153.4 mmol) in 1-methyl-2-pyrrolidinone (300 mL) at 0° C. was slowly added 2-bromoacetyl bromide (30.9 g, 153.4 mmol) dropwise, and the mixture was stirred at room temperature for 16 h. The reaction mixture was diluted with water (160 mL) and MTBE (200 mL), the phases were separated, and the aqueous layer was extracted with MTBE (2×150 mL). The combined organic layers were washed with brine, dried over Na₂SO₄, filtered and concentrated under reduced pressure at 40° C. The residue was purified by flash column chromatography (40 g silica, 0-10% MTBE/petroleum ether) to give 1-(4-(trifluoromethyl)phenyl)cyclobutyl 2-bromoacetate (20 g, 59.3 mmol, 77%) as a yellow oil. ¹H NMR (400 MHz, CDCl₃) δ: 7.67-7.57 (m, 4H), 3.76 (s, 2H), 2.75-2.60 (m, 4H), 2.11-1.98 (m, 1H), 1.85-1.70 (m, 1H).

Step 2

To a solution of methyl 2-(diethoxyphosphoryl)acetate (8.1 g, 38.3 mmol) in THF (150 mL) at 0° C. was added NaH suspension in mineral oil (60 wt. %, 1.5 g, 38.3 mmol) and the reaction mixture was stirred at 0° C. for 0.5 h. 1-(4-(Trifluoromethyl)phenyl)cyclobutyl 2-bromoacetate (10 g, 29.8 mmol) was then added and the reaction mixture was stirred at room temperature overnight. The reaction mixture was quenched with dilute aqueous HCl (0.5 M) and adjusted to pH=5. The phases were separated, and the aqueous layer was extracted with EtOAc (2×100 mL). The combined organic layers were washed with brine, dried over Na₂SO₄, filtered and concentrated under reduced pressure at 40° C. to give 1-methyl 4-(1-(4-(trifluoromethyl)phenyl)cyclobutyl) 2-(diethoxyphosphoryl)succinate (14.4 g, quantitative yield) as a colorless oil, which was used directly in the next step. LCMS (System 2, Method C) m/z 489.0 (M+Na)⁺ (ES⁺).

Step 3

To a mixture of 1-methyl 4-(1-(4-(trifluoromethyl)phenyl)cyclobutyl) 2-(diethoxyphosphoryl)succinate (14.4 g, 30.9 mmol) and potassium carbonate (8.5 g, 61.4 mmol) in THF (200 mL) at room temperature was added formaldehyde solution in water (37 wt. %, 16.3 mL, 153.6 mmol) and the reaction mixture was stirred at room temperature for 4 h. The reaction mixture was diluted with H₂O (150 mL) and extracted with MTBE (2×200 mL). The combined organic layers were washed with H₂O (2×100 mL) and brine, dried over Na₂SO₄, filtered and concentrated under reduced pressure at 30° C. The residue was purified by flash column chromatography (120 g silica, 0-10% MTBE/petroleum ether) to give 1-methyl 4-(1-(4-(trifluoromethyl)phenyl)cyclobutyl) 2-methylenesuccinate (6 g, 17.5 mmol, 56%) as a colorless oil. LCMS (System 2, Method C) m/z 365.0 (M+Na)⁺ (ES⁺).

Step 4

To a solution of 1-methyl 4-(1-(4-(trifluoromethyl)phenyl)cyclobutyl) 2-methylenesuccinate (6 g, 17.6 mmol) in THF (30 mL) was added LiOH solution in water (2 M, 13.5 mL, 26.3 mmol), and the reaction mixture was stirred at room temperature for 6 h and then kept in a fridge at 0° C. overnight. The reaction mixture was acidified with dilute aqueous HCl (0.5 M) until pH=3 and extracted with EtOAc (2×10 mL). The combined EtOAc layers were washed with brine, dried over Na₂SO₄, filtered and concentrated under reduced pressure at 30° C. to give a residue comprising of a 2:1 mixture of 2-methylene-4-oxo-4-(1-(4-(trifluoromethyl)phenyl)cyclobutoxy)butanoic acid and 2-methyl-4-oxo-4-(1-(4-(trifluoromethyl)phenyl)cyclobutoxy)but-2-enoic acid. The mixture was purified by reversed phase column chromatography (330 g C18 silica; flow rate: 60 mL/min; 60-85% MeCN/(10 mM formic acid/water); collection wavelength: 214 nm). The collected fractions were concentrated under reduced pressure at 30° C. to remove MeCN, and the residue was lyophilized to give 2-methylene-4-oxo-4-(1-(4-(trifluoromethyl)phenyl)cyclobutoxy) butanoic acid (1.8 g, 5.48 mmol, 31%, 80% pure) as a white solid. LCMS (System 2, Method B) m/z 351.0 (M+Na)⁺ (ES⁺). ¹H NMR (400 MHz, DMSO-d6) δ: 12.65 (br, 1H), 7.71 (d, J=8.5 Hz, 2H), 7.65 (d, J=8.2 Hz, 2H), 6.13 (d, J=1.6 Hz, 1H), 5.74 (d, J=1.5 Hz, 1H), 3.33 (d, J=3.9 Hz, 2H), 2.61-2.50 (m, 4H), 2.02-1.89 (m, 1H), 1.84-1.69 (m, 1H).

### Preparation of 3-((2,2,2-trichloroethoxy)carbonyl)but-3-enoic acid

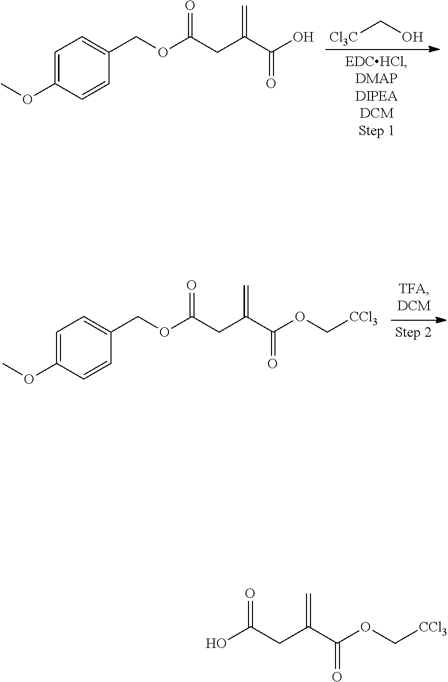

Step 1

To a solution of 4-((4-methoxybenzyl)oxy)-2-methylene-4-oxobutanoic acid (30.0 g, 120 mmol), 2,2,2-trichloroethan-1-ol (19.7 g, 132 mmol), DMAP (11.7 g, 96 mmol) and DIPEA (46.4 g, 360 mmol) in DCM (500 mL) at 0° C. was added EDC·HCl (34.6 g, 180 mmol), and the resulting pale-yellow mixture was stirred at room temperature overnight. The mixture was quenched with dilute aqueous HCl (0.5 M), the phases were separated and the aqueous layer was extracted with DCM (3×500 mL). The combined organic phases were washed with brine, dried over Na₂SO₄ and filtered. The filtrate was concentrated under reduced pressure at 30° C., and the residue was purified by flash column chromatography (120 g silica, 0-20% MTBE/petroleum ether) to give 4-(4-methoxybenzyl) 1-(2,2,2-trichloroethyl) 2-methylenesuccinate (35 g, 91.7 mmol, 76%) as a colorless oil. LCMS: (System 2, Method C) m/z 402.8/404.8 (M+Na)⁺ (ES⁺).

Step 2

A solution of 4-(4-methoxybenzyl) 1-(2,2,2-trichloroethyl) 2-methylenesuccinate (35.0 g, 91.7 mmol) in TFA (40 mL) and DCM (80 mL) was stirred at room temperature for 16 h. The mixture was concentrated under reduced pressure at 30° C. and the residue was purified by reversed phase column chromatography (330 g C18 silica; flow rate: 60 mL/min; 60-80% MeCN/10 mM formic acid/water; collection wavelength: 214 nm). The collected fractions were concentrated under reduced pressure at 30° C. to remove MeCN, and the residue was lyophilized to give 3-((2,2,2-trichloroethoxy)carbonyl)but-3-enoic acid (23.0 g, 88.0 mmol, 96%) as a colorless oil. LCMS: (System 2, Method C) m/z 282.8/284.8 (M+Na)⁺ (ES⁺).

### Preparation of 2-methylene-4-oxo-4-((3-(4-(trifluoromethyl)phenyl)oxetan-3-yl)oxy)butanoic acid

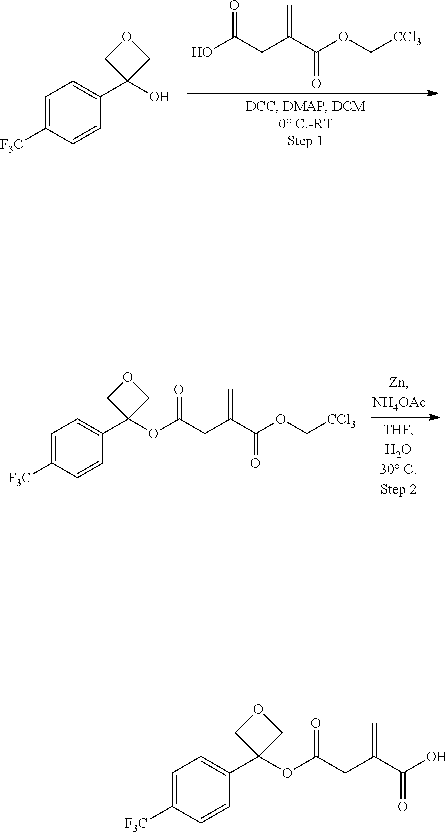

Step 1

A mixture of 3-(4-(trifluoromethyl)phenyl)oxetan-3-ol (330 mg, 1.51 mmol) 3-((2,2,2-trichloroethoxy)carbonyl)but-3-enoic acid (393 mg, 1.51 mmol), DCC (474 mg, 2.3 mmol) and DMAP (20 mg, 0.16 mmol) in DCM (5 mL) was stirred at room temperature for 30 minutes. The reaction mixture was filtered and concentrated under reduced pressure at 35° C. The residue was purified by flash column chromatography (20 g silica, 0-20% MTBE/petroleum ether) to give 1-(2,2,2-trichloroethyl) 4-(3-(4-(trifluoromethyl)phenyl)oxetan-3-yl) 2-methylenesuccinate (560 mg, 80%) as a colorless oil. LCMS (System 2, Method B) m/z 460.8 (M+H)⁺ (ES⁺).

Step 2

A mixture of 1-(2,2,2-trichloroethyl) 4-(3-(4-(trifluoromethyl)phenyl)oxetan-3-yl) 2-methylenesuccinate (280 mg, 0.61 mmol), NH₄OAc (370 mg, 4.8 mmol) and zinc powder (156 mg, 2.4 mmol) in THF (3.2 mL)/H₂O (0.8 mL) was stirred at room temperate for 2 h. The reaction mixture was filtered and the filtrate was acidified with acetic acid to pH=5, and extracted with MTBE (2×5 mL). The combined organic layers were washed with brine, dried over Na₂SO₄, and concentrated under reduced pressure. The residue was purified by preparative HPLC (Column: Waters Sunfire Prep C18 OBD 10 μm 19×250 mm; Flow Rate: 20 mL/min; solvent system: MeCN/(0.05% formic acid/water); gradient: 40-95% MeCN; collection wavelength: 214 nm). The collected fractions were concentrated under reduced pressure at 35° C. to remove MeCN, and the residue was lyophilized to give 2-methylene-4-oxo-4-(3-(4-(trifluoromethyl)phenyl)oxetan-3-yloxy)butanoic acid (131 mg, 66% yield) as white solid. LCMS (System 2, Method B) m/z 331.0 (M+H)⁺ (ES⁺). ¹H NMR (400 MHz, DMSO-d6) δ: 12.81 (s, 1H), 7.80-7.72 (m, 4H), 6.19 (s, 1H), 5.82 (s, 1H), 4.91 (d, J=7.7 Hz, 2H), 4.82 (d, J=7.7 Hz, 2H), 3.50 (s, 2H).

### Preparation of 3-((2-oxo-2-(2,2,2-trichloroethoxy)ethoxy)carbonyl)but-3-enoic

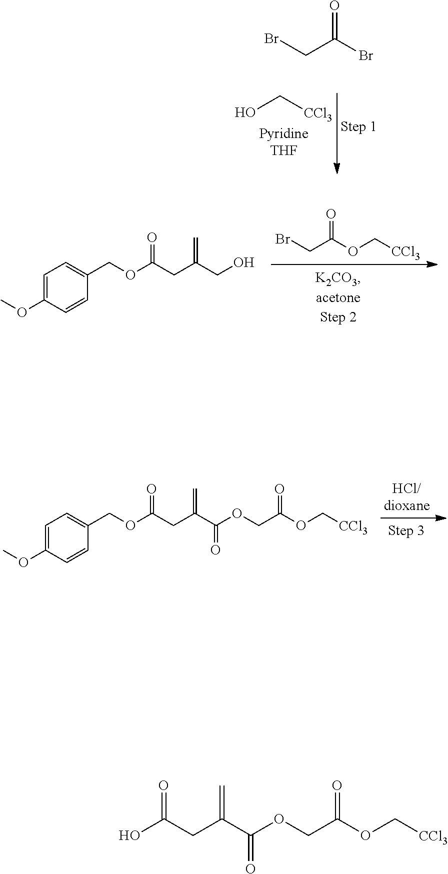

### Step 1

To a solution of ethyl 2,2,2-trichloroethan-1-ol (8.9 g, 60 mmol) and pyridine (6 g, 75 mmol) in THF (150 mL) was added 2-bromoacetyl bromide (10 g, 50 mmol) at 0° C., and the reaction mixture was stirred at room temperature for 4 h. The reaction mixture was diluted with H₂O (400 mL) and extracted with MTBE (3×100 mL). The combined organic layers were washed with 0.5N HCl aq. (2×50 mL) and brine, dried over Na₂SO₄ and concentrated under reduced pressure. The residue was purified by silica gel column chromatography (0-5% MTBE/petroleum ether) to give 2,2,2-trichloroethyl 2-bromoacetate (9.5 g, 71%) as light yellow oil. ¹H NMR (400 MHz, CDCl₃) δ: 4.82 (s, 2H), 3.98 (s, 2H).

Step 2

A mixture of 4-(4-methoxybenzyloxy)-2-methylene-4-oxobutanoic acid (2.50 g, 9.99 mmol), 2,2,2-trichloroethyl 2-bromoacetate (2.70 g, 9.99 mmol) and potassium carbonate (1.52 g, 11.00 mmol) in acetone (50 mL) was stirred at room temperature for 16 h. The mixture was filtered and the filtrate was concentrated under reduced pressure. The residue was purified by flash column chromatography (0-15% MTBE/petroleum ether) to give 4-(4-methoxybenzyl) 1-(2-oxo-2-(2,2,2-trichloroethoxy)ethyl) 2-methylenesuccinate (4.3 g, 98%) as colorless oil. LCMS (System 2, Method B) m/z 461.0 (M+Na)⁺ (ES⁺).

Step 3

A mixture of 4-(4-methoxybenzyl) 1-(2-oxo-2-(2,2,2-trichloroethoxy)ethyl) 2-methylenesuccinate (4.30 g, 9.78 mmol) and HCl/dioxane (4 M, 10 mL) in DCM (10 mL) was stirred at room temperature overnight. The mixture was concentrated under reduced pressure and the residue was purified by reversed column chromatography (Column: Boston ODS 120 g Flash; Flow Rate: 40 mL/min; solvent system: MeCN/(10 mmol/L HCl/water); gradient MeCN: 40-60%; collection wavelength: 214 nm). The fractions were concentrated under reduced pressure to remove MeCN, and the residue was lyophilized to give 3-((2-oxo-2-(2,2,2-trichloroethoxy)ethoxy)carbonyl)but-3-enoic acid (2.50 g, 80%) as colorless oil. LCMS (System 2, Method B) m/z 341.0 (M+Na)⁺ (ES⁺).

### Preparation of Examples

### Example 1—2-((4-(1-(3,5-dichlorophenyl)cyclobutoxy)-2-methylene-4-oxobutanoyl)oxy)acetic acid

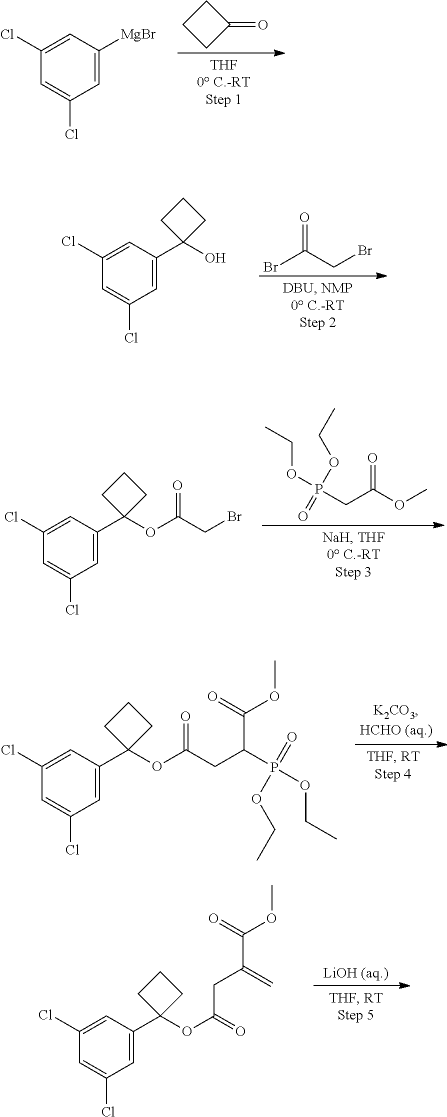

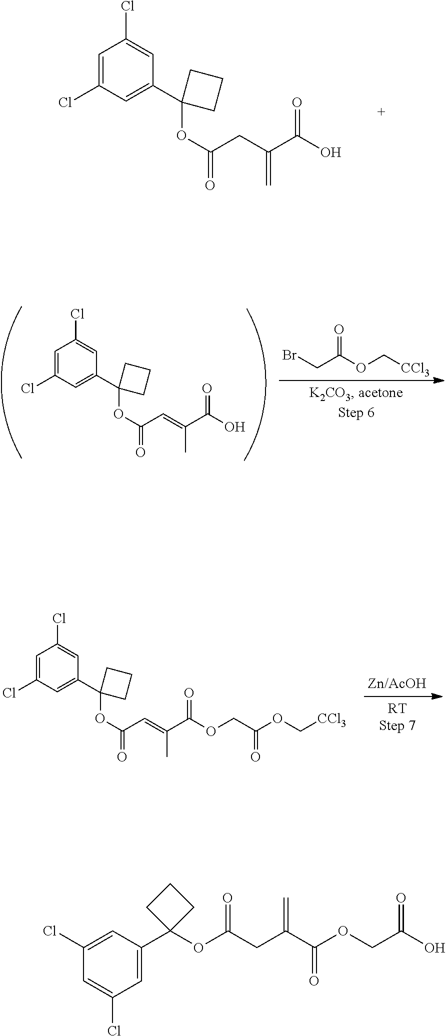

Step 1

To a solution of cyclobutanone (1.40 g, 20.0 mmol) in THF (20 mL) at 0° C. was slowly added a solution of (3,5-dichlorophenyl)magnesium bromide in 2-methyltetrahydrofuran (0.5 M, 20 mL, 10.0 mmol), and the mixture was stirred at room temperature for 16 h. The reaction mixture was quenched with saturated aqueous NH₄Cl solution (30 mL), the phases were separated and the aqueous phase was extracted with ethyl acetate (2×20 mL). The combined organic layers were washed with brine, dried over Na₂SO₄, filtered and concentrated under reduced pressure at 30° C. to give 1-(3,5-dichlorophenyl)cyclobutan-1-ol (2.00 g, 9.21 mmol, 92%) as a colorless oil. ¹H NMR (400 MHz, CDCl₃) δ: 7.38 (d, J=1.9 Hz, 2H), 7.27 (t, J=1.9 Hz, 1H), 2.56-2.45 (m, 2H), 2.41-2.30 (m, 2H), 2.13-2.00 (m, 1H), 1.81-1.69 (m, 1H). One exchangeable proton not observed.

Step 2

To a solution of 1-(3,5-dichlorophenyl)cyclobutan-1-ol (2.00 g, 9.21 mmol) and DBU (2.10 g, 13.8 mmol) in 1-methyl-2-pyrrolidinone (15 mL) at 0° C. was slowly added 2-bromoacetyl bromide (2.79 g, 13.82 mmol) dropwise, and the mixture was stirred at room temperature for 16 h. The reaction mixture was diluted with water (20 mL) and MTBE (30 mL), the phases were separated and the aqueous phase was extracted with MTBE (2×20 mL). The combined organic layers were washed with brine, dried over Na₂SO₄, filtered and concentrated under reduced pressure at 40° C. The residue was purified by flash column chromatography (40 g silica, 0-3% MTBE/petroleum ether) to give 1-(3,5-dichlorophenyl)cyclobutyl 2-bromoacetate (2.80 g, 8.28 mmol, 90%) as a colorless oil. ¹H NMR (400 MHz, CDCl₃) δ: 7.35 (d, J=1.9 Hz, 2H), 7.29 (t, J=1.9 Hz, 1H), 3.76 (s, 2H), 2.68-2.55 (m, 4H), 2.10-1.96 (m, 1H), 1.84-1.70 (m, 1H).

Step 3

To a solution of methyl 2-(diethoxyphosphoryl)acetate (1.74 g, 8.28 mmol) in THF (50 mL) at 0° C. was added NaH suspension in mineral oil (60 wt. %, 332 mg, 8.28 mmol), and the reaction mixture was stirred at 0° C. for 0.5 h. 1-(3,5-dichlorophenyl)cyclobutyl 2-bromoacetate (2.80 g, 8.28 mmol) was then added and the reaction mixture was stirred at room temperature overnight. The reaction mixture was quenched with dilute aqueous HCl (0.5 M) to pH=5, the phases were separated, and the aqueous phase was extracted with EtOAc (2×20 mL). The combined organic layers were washed with brine, dried over Na₂SO₄, filtered and concentrated under reduced pressure at 40° C. to give 4-(1-(3,5-dichlorophenyl)cyclobutyl) 1-methyl 2-(diethoxyphosphoryl)succinate (4.00 g, 8.56 mmol, >100%) as a colorless oil. LCMS: (System 2, Method C) m/z 489.0/491.0 (M+Na)⁺ (ES⁺).

Step 4

To a mixture of 4-(1-(3,5-dichlorophenyl)cyclobutyl) 1-methyl 2-(diethoxyphosphoryl)succinate (4.00 g, 8.56 mmol) and potassium carbonate (2.37 g, 17.1 mmol) in THF (15 mL) at room temperature was added formaldehyde solution in water (37 wt. %, 6.9 mL, 85.6 mmol), and the reaction mixture was stirred at room temperature for 4 h. The reaction mixture was diluted with H₂O (15 mL) and extracted with MTBE (2×20 mL). The combined organic layers were washed with H₂O (2×10 mL) and brine, then dried over Na₂SO₄, filtered and concentrated under reduced pressure at 30° C. The residue was purified by flash column chromatography (40 g silica, 0-10% MTBE/petroleum ether) to give 4-(1-(3,5-dichlorophenyl)cyclobutyl) 1-methyl 2-methylenesuccinate (2.60 g, 7.60 mmol, 88%) as a colorless oil. LCMS: (System 2, Method B) m/z 365.1/367.1 (M+Na)⁺ (ES⁺).

Step 5

To a solution of 4-(1-(3,5-dichlorophenyl)cyclobutyl) 1-methyl 2-methylenesuccinate (2.60 g, 7.60 mmol) in THF (20 mL) was added LiOH solution in water (2 M, 10.5 mL, 21 mmol), and the reaction mixture was stirred at room temperature for 8 h and at 0° C. overnight. The reaction mixture was acidified with dilute aqueous HCl (0.5 M) until pH=3 and extracted with EtOAc (2×30 mL). The combined organic layers were washed with brine, dried over Na₂SO₄, filtered and concentrated under reduced pressure at 30° C. to give a 2:1 mixture of 4-(1-(3,5-dichlorophenyl)cyclobutoxy)-2-methylene-4-oxobutanoic acid and 4-(1-(3,5-dichlorophenyl)cyclobutoxy)-2-methyl-4-oxobut-2-enoic acid (2.30 g, 6.99 mmol, 92%) as a pale yellow oil, which was used directly in the next step. LCMS: (System 2, Method C) m/z 351.2/353.2 (M+Na)⁺ (ES⁺).

Step 6

To a solution of a 2:1 mixture of 4-(1-(3,5-dichlorophenyl)cyclobutoxy)-2-methylene-4-oxobutanoic acid and 4-(1-(3,5-dichlorophenyl)cyclobutoxy)-2-methyl-4-oxobut-2-enoic acid (2.30 g, 6.99 mmol) and potassium carbonate (1.16 g, 8.39 mmol) in acetone (30 mL) was added 2,2,2-trichloroethyl 2-bromoacetate (1.89 g, 6.99 mmol) and the reaction mixture was stirred at room temperature overnight. The reaction mixture was filtered, and the filtrate was concentrated under reduced pressure at 30° C. The residue was purified by flash column chromatography (40 g silica, 0-12% MTBE/petroleum ether) to give 4-(1-(3,5-dichlorophenyl)cyclobutyl) 1-(2-oxo-2-(2,2,2-trichloroethoxy)ethyl) 2-methylenesuccinate (2.00 g, 3.86 mmol, 55%) as a pale yellow oil. LCMS: (System 2, Method C) m/z 535.9/539.0/541.0 (M+Na)⁺ (ES⁺). ¹H NMR (400 MHz, CDCl₃) δ: 7.31 (d, J=1.9 Hz, 2H), 6.46 (s, 1H), 5.80 (s, 1H), 4.83 (s, 2H), 4.80 (s, 2H), 3.35 (s, 2H), 2.58 (dd, J=8.8, 6.9 Hz, 4H), 2.05-1.93 (m, 1H), 1.82-1.68 (m, 1H). One aromatic proton obscured by solvent.

Step 7

To a solution of 4-(1-(3,5-dichlorophenyl)cyclobutyl) 1-(2-oxo-2-(2,2,2-trichloroethoxy)ethyl) 2-methylenesuccinate (2.00 g, 3.86 mmol) in acetic acid (20 mL) was added zinc powder (1.25 g, 19.30 mmol), and the reaction mixture was stirred at room temperature overnight. The reaction mixture was filtered and the filtrate was concentrated under reduced pressure at 30° C. The residue was purified by reversed phase column chromatography (120 g C18 silica; flow rate: 40 mL/min; 60-80% MeCN/(10 mM formic acid/water); collection wavelength: 214 nm). The collected fractions were concentrated under reduced pressure at 15° C. to remove MeCN, and the residue was extracted with MTBE (2×40 mL). The combined organic phase was washed with water and brine, dried over Na₂SO₄, filtered and concentrated under reduced pressure at 15° C. to give 2-((4-(1-(3,5-dichlorophenyl)cyclobutoxy)-2-methylene-4-oxobutanoyl)oxy)acetic acid (954 mg, 2.46 mmol, 64%) as a white solid. LCMS: (System 2, Method B) m/z 409.0/410.9 (M+Na)⁺ (ES⁺). ¹H NMR (400 MHz, DMSO-d6) δ: 13.08 (br, 1H), 7.53 (t, J=1.9 Hz, 1H), 7.41 (d, J=1.9 Hz, 2H), 6.28 (s, 1H), 5.92 (s, 1H), 4.63 (s, 2H), 3.40 (s, 2H), 2.60-2.51 (m, 2H), 2.48-2.42 (m, 2H), 1.98-1.86 (m, 1H), 1.79-1.65 (m, 1H).

The following compounds were prepared using procedures analogous to those reported for Example 1.

| Example | Alcohol used in step 2/Example | |
|---|---|---|
| No. | Structure/Name | LCMS/¹H NMR data |
| 2 | 1-(4-(trifluoromethyl)phenyl)cyclobutan- 1-ol 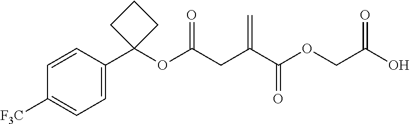 [SMILES: C=C(CC(=O)OC1(c2ccc(C(F)(F)F)cc2)CCC1)C(=O)OCC(=O)O] | LCMS (System 2, Method B) m/z 409.0 (M + Na)⁺ (ES⁺). ¹H NMR (400 MHz, DMSO-d6) δ: 13.12 (br, 1H), 7.72 (d, J = 8.3 Hz, 2H), 7.63 (d, J = 8.3 Hz, 2H), 6.28 (d, J = 1.2 Hz, 1H), 5.91 (d, J = 1.3 Hz, 1H), 4.62 (s, 2H), 3.39 (s, 2H), 2.59- 2.50 (m, 4H), 2.02-1.89 (m, 1H), 1.82- 1.67 (m, 1H). |
| | 2-((2-methylene-4-oxo-4-(1-(4- | |
| | (trifluoromethyl)phenyl)cyclobutoxy) | |
| | butanoyl)oxy)acetic acid | |
| 3 | 1-(4-bromophenyl)cyclobutan-1-ol 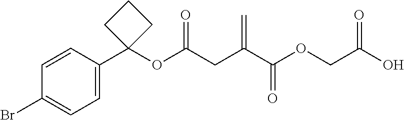 [SMILES: C=C(CC(=O)OC1(c2ccc(Br)cc2)CCC1)C(=O)OCC(=O)O] | LCMS (System 2, Method B) m/z 418.9, 420.9 (M + Na)⁺ (ES⁺). ¹H NMR (400 MHz, DMSO-d6) δ: 13.11 (br, 1H), 7.58-7.50 (m, 2H), 7.41-7.33 (m, 2H), 6.27 (d, J = 1.2 Hz, 1H), 5.89 (d, J = 1.3 Hz, 1H), 4.62 (s, 2H), 3.35 (s, 2H), 2.54-2.51 (m, 2H), 2.49-2.44 (m, 2H), 1.98-1.85 (m, 1H), 1.76-1.60 (m, 1H). |
| | 2-((4-(1-(4-bromophenyl)cyclobutoxy)-2- | |
| | methylene-4-oxobutanoyl)oxy)acetic acid | |
| 4 | 1-(4-chlorophenyl)cyclobutan-1-ol 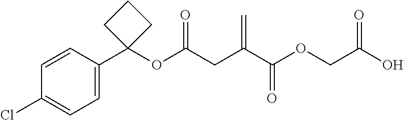 [SMILES: C=C(CC(=O)OC1(c2ccc(Cl)cc2)CCC1)C(=O)OCC(=O)O] | LCMS (System 2, Method B) m/z 375.0 (M + Na)⁺ (ES⁺). ¹H NMR (400 MHz, DMSO-d6) δ: 13.11 (br, 1H), 7.48-7.37 (m, 4H), 6.26 (d, J = 1.3 Hz, 1H), 5.89 (d, J = 1.3 Hz, 1H), 4.62 (s, 2H), 3.35 (s, 2H), 2.55- 2.44 (m, 4H), 1.98-1.85 (m, 1H), 1.76- 1.60 (m, 1H). |
| | 2-((4-(1-(4-chlorophenyl)cyclobutoxy)-2- | |
| | methylene-4-oxobutanoyl)oxy)acetic acid | |
| 5 | 1-(2,5-dichlorophenyl)cyclobutan-1-ol 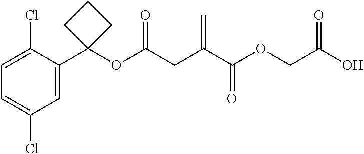 [SMILES: C=C(CC(=O)OC1(c2cc(Cl)ccc2Cl)CCC1)C(=O)OCC(=O)O] | LCMS (System 2, Method B) m/z 408.9/411.0 (M + Na)⁺ (ES⁺). ¹H NMR (400 MHz, DMSO-d6) δ: 13.10 (br, 1H), 7.66 (d, J = 2.5 Hz, 1H), 7.46 (d, J = 8.5 Hz, 1H), 7.40 (dd, J = 8.5, 2.5 Hz, 1H), 6.25 (d, J = 1.3 Hz, 1H), 5.86 (d, J = 1.2 Hz, 1H), 4.58 (s, 2H), 3.30 (s, 2H), 2.80-2.70 (m, 2H), 2.63-2.52 (m, 2H), 2.01-1.88 (m, 1H), 1.69-1.55 (m, 1H). |
| | 2-((4-(1-(2,5- | |
| | dichlorophenyl)cyclobutoxy)-2- | |
| | methylene-4-oxobutanoyl)oxy)acetic acid | |
| 6 | 1-(2-chloro-4- (trifluoromethoxy)phenyl)cyclobutan-1-ol 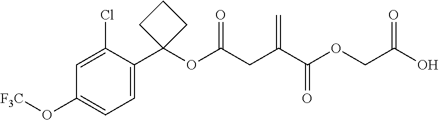 [SMILES: C=C(CC(=O)OC1(c2ccc(OC(F)(F)F)cc2Cl)CCC1)C(=O)OCC(=O)O] | LCMS (System 2, Method B) m/z 459.0 (M + Na)⁺ (ES⁺). ¹H NMR (400 MHz, DMSO-d6) δ: 13.12 (br, 1H), 7.46-7.36 (m, 2H), 7.31-7.25 (m, 1H), 6.27 (d, J = 1.2 Hz, 1H), 5.87 (d, J = 1.3 Hz, 1H), 4.58 (s, 2H), 3.32 (s, 2H), 2.86-2.74 (m, 2H), 2.73-2.63 (m, 2H), 2.16-2.02 (m, 1H), 1.81-1.70 (m, 1H). |
| | 2-((4-(1-(2-chloro-4- | |
| | (trifluoromethoxy)phenyl)cyclobutoxy)-2- | |
| | methylene-4-oxobutanoyl)oxy)acetic acid | |
| 7 | 1-(2,3-dichlorophenyl)cyclobutan-1-ol 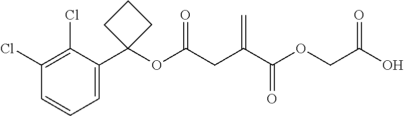 [SMILES: C=C(CC(=O)OC1(c2cccc(Cl)c2Cl)CCC1)C(=O)OCC(=O)O] 2-((4-(1-(2,3- dichlorophenyl)cyclobutoxy)-2- methylene-4-oxobutanoyl)oxy)acetic acid | LCMS (System 2, Method B) m/z 409.0/411.0 (M + Na)⁺ (ESI⁺). ¹H NMR (400 MHz, DMSO-d6) δ: 13.09 (br, 1H), 7.67 (dd, J = 7.9, 1.5 Hz, 1H), 7.59 (dd, J = 8.0, 1.5 Hz, 1H), 7.36 (t, J = 7.9 Hz, 1H), 6.24 (d, J = 1.3 Hz, 1H), 5.86 (d, J = 1.3 Hz, 1H), 4.57 (s, 2H), 3.30 (s, 2H), 2.81-2.70 (m, 2H), 2.65-2.54 (m, 2H), 2.02-1.89 (m, 1H), 1.67-1.53 (m, 1H). |
| 8 | 1-(2,6-dichlorophenyl)cyclobutan-1-ol 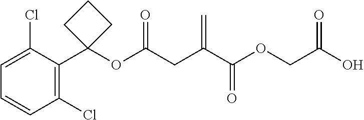 [SMILES: C=C(CC(=O)OC1(c2c(Cl)cccc2Cl)CCC1)C(=O)OCC(=O)O] | LCMS (System 2, Method B) m/z 409.0/410.9 (M + Na)⁺ (ES⁺). ¹H NMR (400 MHz, DMSO-d6) δ: 13.08 (br, 1H), 7.38 (d, J = 8.0 Hz, 2H), 7.26 (t, J = 8 Hz, 1H), 6.27 (d, J = 1.2 Hz, 1H), 5.89 (d, J = 1.3 Hz, 1H), 4.58 (s, 2H), 3.36 (s, 2H), 2.95-2.83 (m, 2H), 2.83-2.73 (m, 2H), 2.14-1.99 (m, 1H), 1.78-1.66 (m, 1H). |
| | 2-((4-(1-(2,6- | |
| | dichlorophenyl)cyclobutoxy)-2- | |
| | methylene-4-oxobutanoyl)oxy)acetic acid | |
| 9 | 1-(4- (trifluoromethoxy)phenyl)cyclobutan-1-ol 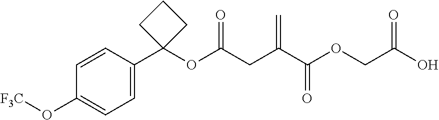 [SMILES: C=C(CC(=O)OC1(c2ccc(OC(F)(F)F)cc2)CCC1)C(=O)OCC(=O)O] | LCMS (System 2, Method B) m/z 425.0 (M + Na)⁺ (ES⁺). ¹H NMR (400 MHz, DMSO-d6) δ: 13.11 (br, 1H), 7.59-7.50 (m, 2H), 7.38-7.31 (m, 2H), 6.27 (d, J = 1.3 Hz, 1H), 5.89 (d, J = 1.3 Hz, 1H), 4.61 (s, 2H), 3.37 (s, 2H), 2.58-2.50 (m, 4H), 1.99-1.86 (m, 1H), 1.78-1.62 (m, 1H). |
| | 2-((2-methylene-4-oxo-4-(1-(4- | |
| | (trifluoromethoxy)phenyl)cyclobutoxy) | |
| | butanoyl)oxy)acetic acid | |
| 10 | 1-(2,4-dichlorophenyl)cyclobutan-1-ol 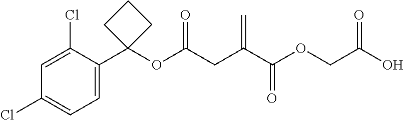 [SMILES: C=C(CC(=O)OC1(c2ccc(Cl)cc2Cl)CCC1)C(=O)OCC(=O)O] | LCMS (System 2, Method B) m/z 408.9/410.9 (M + Na)⁺ (ES⁺). ¹H NMR (400 MHz, DMSO-d6) δ: 13.15 (br, 1H), 7.68 (d, J = 8.5 Hz, 1H), 7.58 (d, J = 2.2 Hz, 1H), 7.41 (dd, J = 8.5, 2.2 Hz, 1H), 6.24 (d, J = 1.3 Hz, 1H), 5.85 (d, J = 1.3 Hz, 1H), 4.58 (s, 2H), 3.29 (s, 2H), 2.78-2.67 (m, 2H), 2.63-2.52 (m, 2H), 2.01-1.88 (m, 1H), 1.67-1.52 (m, 1H). |
| | 2-((4-(1-(2,4- | |
| | dichlorophenyl)cyclobutoxy)-2- | |
| | methylene-4-oxobutanoyl)oxy)acetic acid | |
| 11 | 1-(2-chloro-4- (trifluoromethyl)phenyl)cyclobutan-1-ol 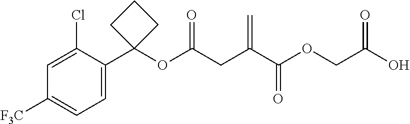 [SMILES: C=C(CC(=O)OC1(c2ccc(C(F)(F)F)cc2Cl)CCC1)C(=O)OCC(=O)O] | LCMS (System 2, Method B) m/z 443.0/445.0 (M + Na)⁺ (ES⁺). ¹H NMR (400 MHz, DMSO-d6) δ: 13.13 (br, 1H), 7.91 (d, J = 8.2 Hz, 1H), 7.84 (d, J = 1.9 Hz, 1H), 7.72 (dd, J = 8.3, 2.0 Hz, 1H), 6.25 (d, J = 1.3 Hz, 1H), 5.86 (d, J = 1.3 Hz, 1H), 4.57 (s, 2H), 3.31 (s, 2H), 2.83-2.72 (m, 2H), 2.67-2.55 (m, 2H), 2.04-1.91 (m, 1H), 1.69-1.56 (m, 1H). |
| | 2-((4-(1-(2-chloro-4- | |
| | (trifluoromethyl)phenyl)cyclobutoxy)-2- | |
| | methylene-4-oxobutanoyl)oxy)acetic acid | |
| 12 | 1-(4-bromo-2-chlorophenyl)cyclobutan-1- ol 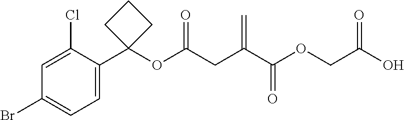 [SMILES: C=C(CC(=O)OC1(c2ccc(Br)cc2Cl)CCC1)C(=O)OCC(=O)O] | LCMS (System 2, Method B) m/z 452.8/454.9 (M + Na)⁺ (ES⁺). ¹H NMR (400 MHz, DMSO-d6) δ: 13.08 (br, 1H), 7.6 (d, J = 2.0 Hz, 1H), 7.62 (d, J = 8.4 Hz, 1H), 7.54 (dd, J = 8.4, 2.0 Hz, 1H), 6.24 (d, J = 1.2 Hz, 1H), 5.85 (d, J = 1.3 Hz, 1H), 4.58 (s, 2H), 3.29 (s, 2H), 2.78-2.66 (m, 2H), 2.63-2.50 (m, 2H), 2.01-1.88 (m, 1H), 1.67-1.52 (m, 1H). |
| | 2-((4-(1-(4-bromo-2- | |
| | chlorophenyl)cyclobutoxy)-2-methylene- | |
| | 4-oxobutanoyl)oxy)acetic acid | |
| 14 | 1-(3,4,5-trifluorophenyl)cyclobutan-1-ol 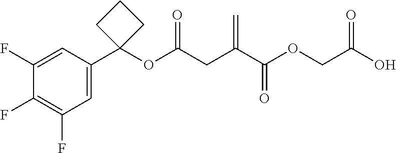 [SMILES: C=C(CC(=O)OC1(c2cc(F)c(F)c(F)c2)CCC1)C(=O)OCC(=O)O] | LCMS (System 2, Method B) m/z 395.0 (M + Na)⁺ (ES⁺). ¹H NMR (400 MHz, DMSO-d6) δ: 13.08 (br, 1H), 7.48-7.35 (m, 1H), 7.35-7.22 (m, 1H), 6.25 (s, 1H), 5.87 (s, 1H), 4.59 (s, 2H), 3.31 (s, 2H), 2.73-2.63 (m, 2H), 2.59-2.50 (m, 2H), 2.00-1.88 (m, 1H), 1.71-1.57 (m, 1H). |
| | 2-((2-methylene-4-oxo-4-(1-(3,4,5- | |
| | trifluorophenyl)cyclobutoxy)butanoyl)oxy) | |
| | acetic acid | |
| 15 | 1-(3-chloro-5- (trifluoromethyl)phenyl)cyclobutan-1-ol 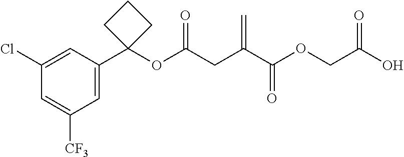 [SMILES: C=C(CC(=O)OC1(c2cc(Cl)cc(C(F)(F)F)c2)CCC1)C(=O)OCC(=O)O] | LCMS (System 2, Method B) m/z 443.0/445.0 (M + Na)⁺ (ES⁺). ¹H NMR (400 MHz, DMSO-d6) δ: 13.11 (br, 1H), 7.79 (s, 1H), 7.77 (s, 1H), 7.65 (s, 1H), 6.28 (s, 1H), 5.92 (s, 1H), 4.62 (s, 2H), 3.40 (s, 2H), 2.65- 2.49 (m, 4H), 2.02-1.89 (m, 1H), 1.81- 1.66 (m, 1H). |
| | 2-((4-(1-(3-chloro-5- | |
| | (trifluoromethyl)phenyl)cyclobutoxy)-2- | |
| | methylene-4-oxobutanoyl)oxy)acetic acid | |
| 16 | 1-(3-chloro-4- (trifluoromethyl)phenyl)cyclobutan-1-ol 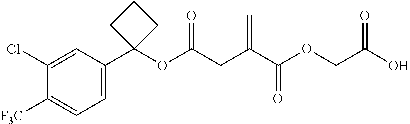 [SMILES: C=C(CC(=O)OC1(c2ccc(C(F)(F)F)c(Cl)c2)CCC1)C(=O)OCC(=O)O] | LCMS (System 2, Method B) m/z 442.9/444.8 (M + Na)⁺ (ES⁺). ¹H NMR (400 MHz, DMSO-d6) δ: 7.84 (d, J = 8.3 Hz, 1H), 7.69 (d, J = 1.8 Hz, 1H), 7.58 (d, J = 8.2 Hz, 1H), 6.28 (s, 1H), 5.91 (s, 1H), 4.59 (s, 2H), 3.41 (s, 2H), 2.62-2.50 (m, 4H), 2.02-1.88 (m, 1H), 1.83-1.69 (m, 1H). One exchangeable proton not observed. |
| | 2-((4-(1-(3-chloro-4- | |
| | (trifluoromethyl)phenyl)cyclobutoxy)-2- | |
| | methylene-4-oxobutanoyl)oxy)acetic acid | |
| 17 | 1-(3,5-dichloro-4- fluorophenyl)cyclobutan-1-ol 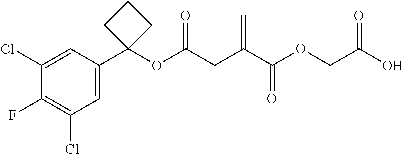 [SMILES: C=C(CC(=O)OC1(c2cc(Cl)c(F)c(Cl)c2)CCC1)C(=O)OCC(=O)O] | LCMS (System 2, Method B) m/z 426.9/428.8 (M + Na)⁺ (ES⁺). ¹H NMR (400 MHz, DMSO-d6) δ: 13.12 (br, 1H), 7.59 (d, J = 6.3 Hz, 2H), 6.28 (d, J = 1.2 Hz, 1H), 5.92 (d, J = 1.3 Hz, 1H), 4.63 (s, 2H), 3.39 (s, 2H), 2.62-2.52 (m, 2H), 2.50-2.41 (m, 2H), 1.98-1.84 (m, 1H), 1.78-1.62 (m, 1H). |
| | 2-((4-(1-(3,5-dichloro-4- | |
| | fluorophenyl)cyclobutoxy)-2-methylene- | |
| | 4-oxobutanoyl)oxy)acetic acid | |
| 18 | 1-(3,5-dichloro-4- methoxyphenyl)cyclobutan-1-ol 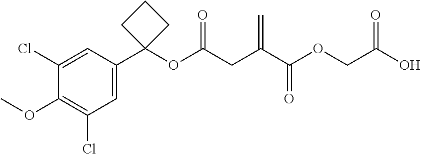 [SMILES: C=C(CC(=O)OC1(c2cc(Cl)c(OC)c(Cl)c2)CCC1)C(=O)OCC(=O)O] | LCMS (System 2, Method B) m/z 438.8/440.8 (M + Na)⁺ (ES⁺). ¹H NMR (400 MHz, DMSO-d6) δ: 13.13 (br, 1H), 7.48 (s, 2H), 6.28 (d, J = 1.3 Hz, 1H), 5.92 (d, J = 1.3 Hz, 1H), 4.62 (s, 2H), 3.82 (s, 3H), 3.38 (s, 2H), 2.60-2.50 (m, 2H), 2.49-2.40 (m, 2H), 1.96-1.84 (m, 1H), 1.76-1.62 (m, 1H). |
| | 2-((4-(1-(3,5-dichloro-4- | |
| | methoxyphenyl)cyclobutoxy)-2- | |
| | methylene-4-oxobutanoyl)oxy)acetic acid | |
| 19 | 1-(3,5-difluoro-4- (trifluoromethyl)phenyl)cyclobutan-1-ol 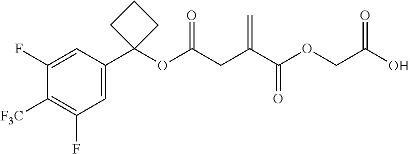 [SMILES: C=C(CC(=O)OC1(c2cc(F)c(C(F)(F)F)c(F)c2)CCC1)C(=O)OCC(=O)O] | LCMS (System 2, Method B) m/z 445.0 (M + Na)⁺ (ES⁺). ¹H NMR (400 MHz, DMSO-d6) δ: 12.97 (br, 1H), 7.41 (d, J = 11.5 Hz, 2H), 6.30 (d, J = 1.2 Hz, 1H), 5.95 (d, J = 1.2 Hz, 1H), 4.64 (s, 2H), 3.45 (s, 2H), 2.61-2.49 (m, 4H), 2.00-1.87 (m, 1H), 1.87-1.73 (m, 1H). |
| | 2-((4-(1-(3,5-difluoro-4- | |
| | (trifluoromethyl)phenyl)cyclobutoxy)-2- | |
| | methylene-4-oxobutanoyl)oxy)acetic acid | |
| 20 | 1-(3-chloro-4-methylphenyl)cyclobutan- 1-ol 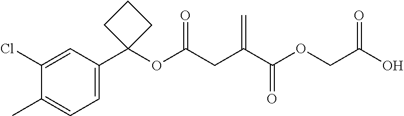 [SMILES: C=C(CC(=O)OC1(c2ccc(C)c(Cl)c2)CCC1)C(=O)OCC(=O)O] | LCMS (System 2, Method B) m/z 389.0/391.0 (M + Na)⁺ (ES⁺). ¹H NMR (400 MHz, DMSO-d6) δ: 13.08 (br, 1H), 7.39 (d, J = 1.8 Hz, 1H), 7.33 (d, J = 8.0 Hz, 1H), 7.28 (dd, J = 7.9, 1.9 Hz, 1H), 6.26 (d, J = 1.3 Hz, 1H), 5.89 (d, J = 1.4 Hz, 1H), 4.62 (s, 2H), 3.35 (s, 2H), 2.57-2.42 (m, 4H), 2.31 (s, 3H), 1.97-1.84 (m, 1H), 1.74-1.59 (m, 1H). |
| | 2-((4-(1-(3-chloro-4- | |
| | methylphenyl)cyclobutoxy)-2-methylene- | |
| | 4-oxobutanoyl)oxy)acetic acid | |

### Example 13—3-((4-(1-(3,5-dichlorophenyl)cyclobutoxy)-2-methylene-4-oxobutanoyl)oxy)propanoic acid

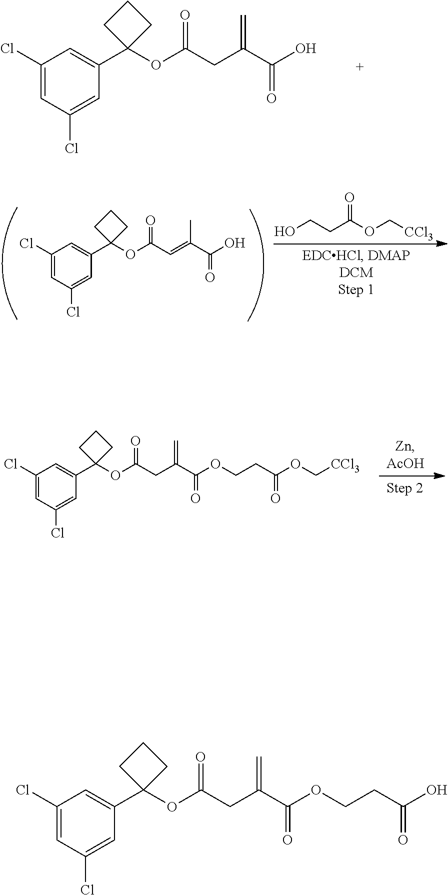

Step 1

To a solution of a 2:1 mixture of 4-(1-(3,5-dichlorophenyl)cyclobutoxy)-2-methylene-4-oxobutanoic acid and 4-(1-(3,5-dichlorophenyl)cyclobutoxy)-2-methyl-4-oxobut-2-enoic acid (Example 1 Step 5, 280 mg, 0.85 mmol), 2,2,2-trichloroethyl 3-hydroxypropanoate (188 mg, 0.85 mmol), DIPEA (329 mg, 2.55 mmol) and DMAP (83 mg, 0.68 mmol) in DCM (5 mL) at 0° C. was added EDC·HCl (245 mg, 1.27 mmol), and the resulting pale yellow mixture was stirred at room temperature for 2 h. The mixture was quenched with dilute aqueous HCl (0.5 M) and to adjusted pH=6, the phases were separated, and the aqueous phase was extracted with DCM (2×5 mL). The combined organic layers were washed with brine, dried over Na₂SO₄ and filtered. The filtrate was concentrated under reduced pressure at 30° C., and the residue was purified by flash column chromatography (25 g silica, 0-6% MTBE/petroleum ether) to give 4-(1-(3,5-dichlorophenyl)cyclobutyl) 1-(3-oxo-3-(2,2,2-trichloroethoxy)propyl) 2-methylenesuccinate (190 mg, 0.36 mmol, 42%) as a colorless oil. LCMS: (System 2, Method B) m/z 554.8/556.8 (M+Na)+(ES⁺).

Step 2

A mixture of 4-(1-(3,5-dichlorophenyl)cyclobutyl) 1-(3-oxo-3-(2,2,2-trichloroethoxy)propyl) 2-methylenesuccinate (190 mg, 0.36 mmol) and zinc powder (117 mg, 1.80 mmol) in AcOH (3 mL) was stirred at room temperature for 2 days. The mixture was filtered, the filtrate was concentrated under reduced pressure at 20° C. and the residue was purified by preparative HPLC (Column: Waters X-Bridge C18 OBD 10 μm 19×250 mm; Flow Rate: 20 mL/min; solvent system: MeCN/(0.2% formic acid/water); gradient: 57-95% MeCN; collection wavelength: 214 nm). The collected fractions were concentrated under reduced pressure at 20° C. to remove MeCN, and the residue was lyophilized to give still impure product (80 mg) which was purified further by flash column chromatography (25 g silica, 0-21% MTBE/petroleum ether) to give 3-((4-(1-(3,5-dichlorophenyl)cyclobutoxy)-2-methylene-4-oxobutanoyl)oxy)propanoic acid (58 mg, 0.14 mmol, 39%) as a colorless oil. LCMS: (System 2, Method B) m/z 423.0/425.0 (M+Na)⁺ (ES⁺). ¹H NMR (400 MHz, DMSO-d6) δ: 12.40 (br, 1H), 7.53 (t, J=1.9 Hz, 1H), 7.42 (d, J=1.9 Hz, 2H), 6.16 (d, J=1.4 Hz, 1H), 5.83 (d, J=1.3 Hz, 1H), 4.25 (t, J=6.3 Hz, 2H), 3.36 (s, 2H), 2.62-2.41 (m, 6H), 2.00-1.87 (m, 1H), 1.79-1.66 (m, 1H).

### Example 21—(S)-4,4,4-trifluoro-3-((2-methylene-4-oxo-4-(1-(4-(trifluoromethyl)phenyl)cyclobutoxy)butanoyl)oxy)butanoic acid

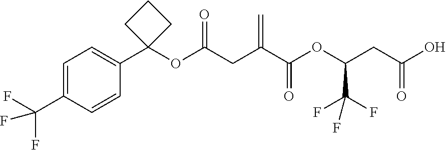 [SMILES: C=C(CC(=O)OC1(c2ccc(C(F)(F)F)cc2)CCC1)C(=O)O[C@@H](CC(=O)O)C(F)(F)F]

Prepared by an analogous method to Example 13 using a mixture of 2-methylene-4-oxo-4-(1-(4-(trifluoromethyl)phenyl)cyclobutoxy)butanoic acid and 2-methyl-4-oxo-4-(1-(4-(trifluoromethyl)phenyl)cyclobutoxy)but-2-enoic acid (342 mg, 1.04 mmol) (formed during the preparation of Example 2) and 2,2,2-trichloroethyl (S)-4,4,4-trifluoro-3-hydroxybutanoate (300 mg, 1.04 mmol). The crude product was purified by preparative HPLC (Column: Waters Sunfire Prep C18 OBD 10 μm 19×250 mm; Flow Rate: 20 mL/min; solvent system: MeCN/(0.2% formic acid/water); gradient: 60-95% MeCN; collection wavelength: 214 nm). The collected fractions were concentrated under reduced pressure at 35° C. to remove MeCN and the aqueous residue was lyophilized to give (S)-4,4,4-trifluoro-3-((2-methylene-4-oxo-4-(1-(4-(trifluoromethyl)phenyl)cyclobutoxy)butanoyl)oxy)butanoic acid (63 mg) as a white solid. LCMS: (System 2, Method B) m/z 490.9 (M+Na)⁺ (ES⁺). ¹H NMR (400 MHz, DMSO-d₆) δ: 12.82 (br, 1H), 7.71 (d, J=8.3 Hz, 2H), 7.64 (d, J=8.2 Hz, 2H), 6.29 (s, 1H), 5.97 (s, 1H), 5.83-5.72 (m, 1H), 3.40 (s, 2H), 2.97-2.88 (m, 1H), 2.78-2.69 (m, 1H), 2.62-2.50 (m, 4H), 2.01-1.88 (m, 1H), 1.81-1.67 (m, 1H).

### Example 22—3-((2-methylene-4-oxo-4-(1-(4-(trifluoromethyl)phenyl)cyclobutoxy)butanoyl)oxy)propanoic acid

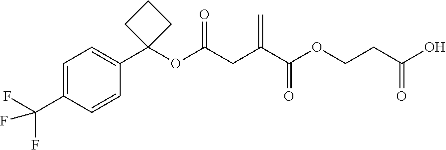 [SMILES: C=C(CC(=O)OC1(c2ccc(C(F)(F)F)cc2)CCC1)C(=O)OCCC(=O)O]

Prepared by an analogous method to Example 13 using a mixture of 2-methylene-4-oxo-4-(1-(4-(trifluoromethyl)phenyl)cyclobutoxy)butanoic acid and 2-methyl-4-oxo-4-(1-(4-(trifluoromethyl)phenyl)cyclobutoxy)but-2-enoic acid (295 mg, 0.90 mmol) (formed during the preparation of Example 2) and 2,2,2-trichloroethyl 3-hydroxypropanoate (200 mg, 0.90 mmol). The crude product was purified by preparative HPLC (Column: Waters Sunfire Prep C18 OBD 10 μm 19×250 mm; Flow Rate: 20 mL/min; solvent system: MeCN/(0.2% formic acid/water); gradient: 55-95% MeCN; collection wavelength: 214 nm). The collected fractions were concentrated under reduced pressure at 25° C. to remove MeCN and the aqueous residue was lyophilized to give 3-((2-methylene-4-oxo-4-(1-(4-(trifluoromethyl)phenyl)cyclobutoxy)butanoyl)oxy)propanoic acid (66 mg) as a white solid. LCMS: (System 2, Method B) m/z 422.8 (M+Na)⁺ (ES⁺). ¹H NMR (400 MHz, DMSO-d6) δ: 12.40 (br, 1H), 7.73 (d, J=8.2 Hz, 2H), 7.64 (d, J=8.2 Hz, 2H), 6.15 (d, J=1.4 Hz, 1H), 5.81 (d, J=1.4 Hz, 1H), 4.23 (t, J=6.2 Hz, 2H), 3.36 (s, 2H), 2.62-2.51 (m, 6H), 2.03-1.89 (m, 1H), 1.83-1.67 (m, 1H).

### Example 23—(R)-4,4,4-trifluoro-3-((2-methylene-4-oxo-4-(1-(4-(trifluoromethyl)phenyl)cyclobutoxy)butanoyl)oxy)butanoic acid

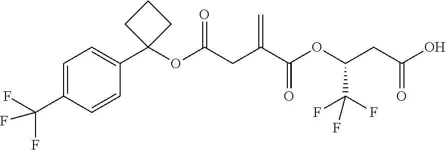 [SMILES: C=C(CC(=O)OC1(c2ccc(C(F)(F)F)cc2)CCC1)C(=O)O[C@H](CC(=O)O)C(F)(F)F]

Prepared by an analogous method to Example 21 using 2,2,2-trichloroethyl (R)-4,4,4-trifluoro-3-hydroxybutanoate (380 mg, 1.32 mmol). The crude product was purified by preparative HPLC (Column: Waters Sunfire Prep C18 OBD 10 μm 19×250 mm; Flow Rate: 20 mL/min; solvent system: MeCN/(0.2% formic acid/water); gradient: 60-95% MeCN; collection wavelength: 214 nm). The collected fractions were concentrated under reduced pressure at 35° C. to remove MeCN and the aqueous residue was lyophilized to give (R)-4,4,4-trifluoro-3-((2-methylene-4-oxo-4-(1-(4-(trifluoromethyl)phenyl)cyclobutoxy)butanoyl)oxy)butanoic acid (23 mg) as a colorless oil. LCMS: (System 2, Method B) m/z 490.9 (M+Na)⁺ (ES⁺). ¹H NMR (400 MHz, CDCl₃) δ: 7.64-7.51 (m, 4H), 6.37 (s, 1H), 5.87-5.77 (m, 1H), 5.77 (s, 1H), 3.31 (s, 2H), 2.85 (dd, J=17.1, 4.2 Hz, 1H), 2.76 (dd, J=17.0, 8.8 Hz, 1H), 2.69-2.54 (m, 4H), 2.07-1.94 (m, 1H), 1.82-1.68 (m, 1H). One exchangeable proton not observed.

### Example 24—2-((4-(1-(4-chlorobenzyl)cyclobutoxy)-2-methylene-4-oxobutanoyl)oxy)acetic acid

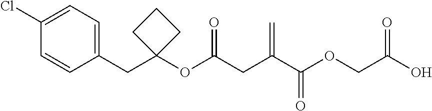 [SMILES: C=C(CC(=O)OC1(Cc2ccc(Cl)cc2)CCC1)C(=O)OCC(=O)O]

Prepared by an analogous procedure to Example 1 starting from 1-(4-chlorobenzyl)cyclobutan-1-ol (1.50 g, 7.63 mmol) in Step 2, except that IPA was used as solvent in place of THF in Step 5. The crude product was purified by preparative HPLC (Column: Waters Sunfire Prep C18 OBD 10 μm 19×250 mm; Flow Rate: 20 mL/min; solvent system: MeCN/(0.2% formic acid/water); gradient: 53-95% MeCN; collection wavelength: 214 nm). The collected fractions were concentrated under reduced pressure at 30° C. to remove MeCN and the aqueous residue was lyophilized to give 2-((4-(1-(4-chlorobenzyl)cyclobutoxy)-2-methylene-4-oxobutanoyl)oxy)acetic acid (188 mg) as a colorless oil. LCMS: (System 2, Method B) m/z 388.9/390.9 (M+Na)⁺ (ES⁺). ¹H NMR (400 MHz, DMSO-d6) δ: 13.07 (br, 1H), 7.37-7.31 (m, 2H), 7.23-7.17 (m, 2H), 6.28 (d, J=1.3 Hz, 1H), 5.91 (d, J=1.3 Hz, 1H), 4.65 (s, 2H), 3.33 (s, 2H), 3.15 (s, 2H), 2.22-2.08 (m, 4H), 1.81-1.69 (m, 1H), 1.69-1.54 (m, 1H).

### Example 25—2-((2-methylene-4-oxo-4-((1-(4-(trifluoromethyl)phenyl)cyclopentyl)oxy)butanoyl)oxy)acetic acid

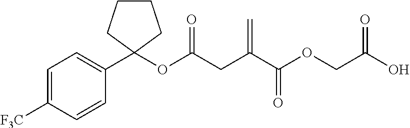 [SMILES: C=C(CC(=O)OC1(c2ccc(C(F)(F)F)cc2)CCCC1)C(=O)OCC(=O)O]

Prepared by an analogous procedure to Example 24 starting from 1-(4-(trifluoromethyl)phenyl)cyclopentan-1-ol (3.10 g, 13.5 mmol) in Step 2, except that Step 5 was stirred at 10-15° C. for 2 h. The crude product was purified by preparative HPLC (Column: Waters Sunfire Prep C18 OBD 10 μm 19×250 mm; Flow Rate: 20 mL/min; solvent system: MeCN/(0.2% TFA/water); gradient: 53-95% MeCN; collection wavelength: 214 nm). The collected fractions were concentrated under reduced pressure at 30° C. to remove MeCN and the aqueous residue was lyophilized to give 2-((2-methylene-4-oxo-4-((1-(4-(trifluoromethyl)phenyl)cyclopentyl)oxy)butanoyl)oxy)acetic acid (114 mg) as a colorless oil. LCMS: (System 2, Method B) m/z 423.0 (M+Na)⁺ (ES⁺). ¹H NMR (400 MHz, DMSO-d6) δ: 13.11 (br, 1H), 7.67 (d, J=8.1 Hz, 2H), 7.54 (d, J=8.1 Hz, 2H), 6.27 (d, J=1.3 Hz, 1H), 5.91 (d, J=1.3 Hz, 1H), 4.64 (s, 2H), 3.39 (s, 2H), 2.36-2.20 (m, 2H), 2.12-1.95 (m, 2H), 1.85-1.66 (m, 4H).

### Example 26—2-((4-(1-(4′-fluoro-[1,1′-biphenyl]-4-yl)cyclobutoxy)-2-methylene-4-oxobutanoyl)oxy)acetic acid

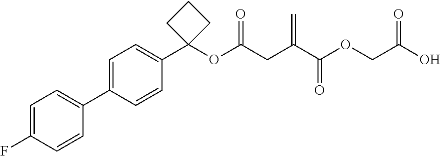 [SMILES: C=C(CC(=O)OC1(c2ccc(-c3ccc(F)cc3)cc2)CCC1)C(=O)OCC(=O)O]

Prepared by an analogous procedure to Example 24 from Step 2 through to Step 6, starting from 1-(4′-fluoro-[1,1′-biphenyl]-4-yl)cyclobutan-1-ol (4.0 g, 16.5 mmol). A modified Step 7 was then used: the product of Step 6, 4-(1-(4′-fluoro-[1,1′-biphenyl]-4-yl)cyclobutyl) 1-(2-oxo-2-(2,2,2-trichloroethoxy)ethyl) 2-methylenesuccinate (410 mg, 0.76 mmol) and ammonium acetate (468 mg, 6.08 mmol) in THF:H2O (4:1, 3 mL) was treated with zinc powder (247 mg, 3.80 mmol), and the reaction mixture was stirred at room temperature overnight. The reaction mixture was filtered, and the filtrate was acidified with dilute aqueous HCl (0.5 M) until pH=3 and extracted with MTBE (2×5 mL). The combined organic layers were washed with brine, dried over Na₂SO₄, filtered and concentrated at 30° C. under reduced pressure. The residue was purified by preparative HPLC (Column: Waters Sunfire Prep C18 OBD 10 μm 19×250 mm; Flow Rate: 20 mL/min; solvent system: MeCN/(0.2% formic acid/water); gradient: 60-95% MeCN; collection wavelength: 214 nm). The collected fractions were concentrated under reduced pressure at 35° C. to remove MeCN and the aqueous residue was lyophilized to give 2-((4-(1-(4′-fluoro-[1,1′-biphenyl]-4-yl)cyclobutoxy)-2-methylene-4-oxobutanoyl)oxy)acetic acid (152 mg, 0.37 mmol, 49%) as a white solid. LCMS: (System 2, Method B) m/z 435.0 (M+Na)⁺ (ES⁺). ¹H NMR (400 MHz, DMSO-d6) δ: 13.13 (br, 1H), 7.74-7.66 (m, 2H), 7.66-7.58 (m, 2H), 7.54-7.46 (m, 2H), 7.34-7.24 (t, J=8.8 Hz, 2H), 6.27 (s, 1H), 5.89 (s, 1H), 4.61 (s, 2H), 3.37 (s, 2H), 2.63-2.50 (m, 4H), 2.01-1.86 (m, 1H), 1.80-1.65 (m, 1H).

### Example 27—2-((2-methylene-4-oxo-4-(1-(4-(trifluoromethyl)benzyl)cyclopropoxy)butanoyl)oxy)acetic acid

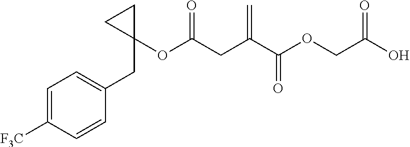 [SMILES: C=C(CC(=O)OC1(Cc2ccc(C(F)(F)F)cc2)CC1)C(=O)OCC(=O)O]

Prepared by an analogous procedure to Example 26 starting from 1-(4-(trifluoromethyl)benzyl)cyclopropan-1-ol (6.0 g, 27.8 mmol) in Step 2, except that Step 5 was stirred at 10° C. for 2 h and Step 7 was stirred at 30° C. for 2 h. The crude product was purified by preparative HPLC (Column: Waters Sunfire Prep C18 OBD 10 μm 19×250 mm; Flow Rate: 20 mL/min; solvent system: MeCN/(0.2% formic acid/water); gradient: 53-95% MeCN; collection wavelength: 214 nm). The collected fractions were concentrated under reduced pressure at 35° C. to remove MeCN and the aqueous residue was lyophilized to give 2-((2-methylene-4-oxo-4-(1-(4-(trifluoromethyl)benzyl)cyclopropoxy)butanoyl)oxy)acetic acid (26 mg) as a colorless oil. LCMS: (System 2, Method B) m/z 387.0 (M+H)⁺ (ES⁺). ¹H NMR (400 MHz, DMSO-d6) δ: 13.05 (br, 1H), 7.66 (d, J=8.0 Hz, 2H), 7.44 (d, J=8.0 Hz, 2H), 6.26 (s, 1H), 5.88 (s, 1H), 4.61 (s, 2H), 3.27 (s, 2H), 3.11 (s, 2H), 0.87-0.78 (m, 4H).

### Example 28—2-((2-methylene-4-oxo-4-(1-((4-(trifluoromethyl)phenyl)ethynyl)cyclobutoxy)butanoyl)oxy)acetic acid

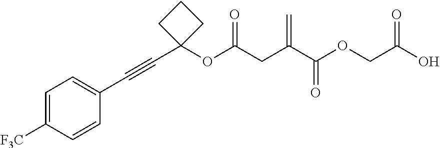 [SMILES: C=C(CC(=O)OC1(C#Cc2ccc(C(F)(F)F)cc2)CCC1)C(=O)OCC(=O)O]

Prepared by an analogous procedure to Example 27 starting from 1-((4-(trifluoromethyl)phenyl)ethynyl)cyclobutyl 2-bromoacetate (5.9 g, 16.3 mmol) in Step 3. The crude product was purified by preparative HPLC (Column: Waters X-Bridge C18 OBD 10 μm 19×250 mm; Flow Rate: 20 mL/min; solvent system: MeCN/(0.2% formic acid/water); gradient: 58-95% MeCN; collection wavelength: 214 nm). The collected fractions were concentrated under reduced pressure at 30° C. to remove MeCN and the aqueous residue was lyophilized to give 2-((2-methylene-4-oxo-4-(1-((4-(trifluoromethyl)phenyl)ethynyl)cyclobutoxy)butanoyl)oxy)acetic acid (116 mg) as a colorless oil. LCMS: (System 2, Method B) m/z 432.9 (M+Na)⁺ (ES⁺). ¹H NMR (400 MHz, DMSO-d6) δ: 13.09 (br, 1H), 7.75 (d, J=8.0 Hz, 2H), 7.64 (d, J=8.0 Hz, 2H), 6.32 (s, 1H), 5.97 (s, 1H), 4.66 (s, 2H), 3.43 (s, 2H), 2.62-2.39 (m, 4H), 1.99-1.85 (m, 2H).

### Example 29—2-((2-methylene-4-oxo-4-((3-(4-(trifluoromethyl)phenyl)oxetan-3-yl)oxy)butanoyl)oxy)acetic acid

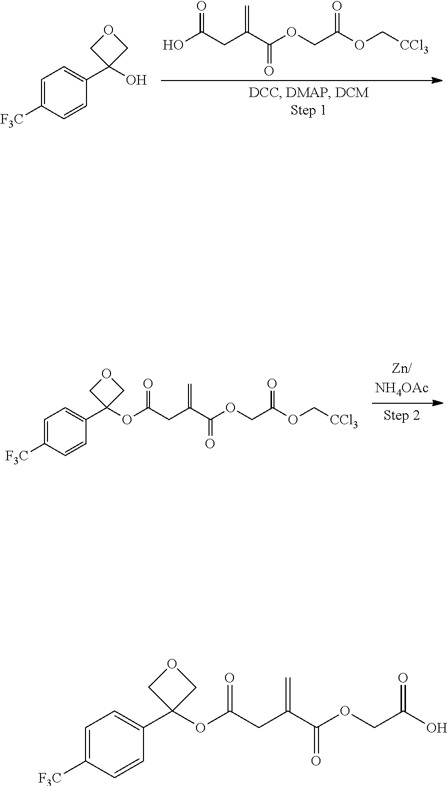

Step 1

A mixture of 3-(4-(trifluoromethyl)phenyl)oxetan-3-ol (200 mg, 0.91 mmol) 3-((2,2,2-trichloroethoxy)carbonyl)but-3-enoic acid (292 mg, 0.91 mmol), DCC (932 mg, 4.55 mmol) and DMAP (12 mg, 0.10 mmol) in DCM (3 mL) was stirred at room temperature for 30 minutes. The mixture was filtered and the filtrate was concentrated under reduced pressure. The residue was purified by flash column chromatography (0-20% MTBE/petroleum ether) to give 1-(2-oxo-2-(2,2,2-trichloroethoxy)ethyl) 4-(3-(4-(trifluoromethyl)phenyl)oxetan-3-yl) 2-methylenesuccinate (350 mg, 73%) as a colorless oil. LCMS: (System 2, Method C) m/z 540.7 (M+Na)⁺ (ES⁺).

Step 2

A mixture of 1-(2-oxo-2-(2,2,2-trichloroethoxy)ethyl) 4-(3-(4-(trifluoromethyl)phenyl)oxetan-3-yl) 2-methylenesuccinate (350 mg, 0.67 mmol), NH₄OAc (413 mg, 5.36 mmol) and zinc powder (217 mg, 3.35 mmol) in THF (3 mL) and H₂O (0.5 mL) was stirred at 30° C. for 3 h. The reaction mixture was filtered and the filtrate was acidified with 0.5N HCl until pH=3-4, and extracted with ethyl acetate (3×10 mL). The ethyl acetate layer was washed by brine, dried over Na₂SO₄ and concentrated under reduced pressure. The residue was purified by prep-HPLC (Column: Waters SUNFIRE Prep C18 OBD 10 μm 19×250 mm; Flow Rate: 20 mL/min; solvent system: MeCN/(0.2% formic acid/water) gradient MeCN: 40-95%; collection wavelength: 214 nm). The fractions were concentrated under reduced pressure to remove MeCN, and the residue was lyophilized to give 2-((2-methylene-4-oxo-4-((3-(4-(trifluoromethyl)phenyl)oxetan-3-yl)oxy)butanoyl)oxy)acetic acid (107 mg, 41%) as white solid. LCMS (System 2, Method B) m/z 410.9 (M+Na)⁺ (ES⁺). ¹H NMR (400 MHz, DMSO-d6) δ: 13.16 (br, 1H), 7.79 (d, J=8.4 Hz, 2H), 7.72 (d, J=8.4 Hz, 2H), 6.33 (d, J=0.8 Hz, 1H), 5.99 (d, J=0.8 Hz, 1H), 4.93 (d, J=8.0 Hz, 2H), 4.81 (d, J=8.0 Hz, 2H), 4.66 (s, 2H), 3.58 (s, 2H).

### Example 30—2-((2-methylene-4-oxo-4-(1-(4-(trifluoromethyl)phenyl) cyclopropoxy)butanoyl)oxy)acetic acid

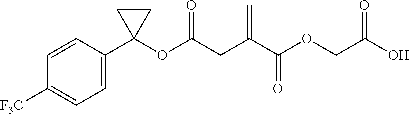 [SMILES: C=C(CC(=O)OC1(c2ccc(C(F)(F)F)cc2)CC1)C(=O)OCC(=O)O]

Prepared by an analogous procedure to Example 29 starting from 1-(4-(trifluoromethyl)phenyl)cyclopropanol. The crude product was purified by preparative HPLC (Column: Waters SUNFIRE Prep C18 OBD 10 μm 19×250 mm; Flow Rate: 20 mL/min; solvent system: MeCN/(0.2% formic acid/water) gradient: MeCN: 40-95%; collection wavelength: 214 nm). The fractions were concentrated under reduced pressure to remove MeCN, and the residue was lyophilized to give 2-((2-methylene-4-oxo-4-(1-(4-(trifluoromethyl)phenyl) cyclopropoxy)butanoyl)oxy)acetic acid (68 mg, 39%) as white solid. LCMS (System 2, Method B) m/z 372.9 (M+H)⁺ (ES⁺). ¹H NMR (400 MHz, DMSO-d6) δ: 13.15 (s, 1H), 7.65 (d, J=8.4 Hz, 2H), 7.35 (d, J=8.4 Hz, 2H), 6.32 (d, J=1.2 Hz, 1H), 5.97 (d, J=0.8 Hz, 1H), 4.66 (s, 2H), 3.49 (s, 2H), 1.35 (s, 4H).

### Example 31—2-((4-(3,3-difluoro-1-(4-(trifluoromethyl)phenyl)cyclobutoxy)-2-methylene-4-oxobutanoyl)oxy)acetic acid

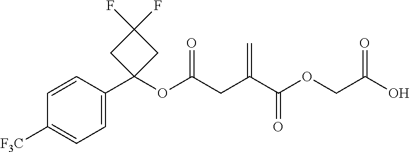 [SMILES: C=C(CC(=O)OC1(c2ccc(C(F)(F)F)cc2)CC(F)(F)C1)C(=O)OCC(=O)O]

Prepared by an analogous procedure to Example 29 starting from 3,3-difluoro-1-(4-(trifluoromethyl)phenyl)cyclobutan-1-ol. The crude product was purified by preparative HPLC (Column: Waters X-Bridge C18 OBD 10 μm 19×250 mm; Flow Rate: 20 mL/min; solvent system: MeCN/(0.2% formic acid/water); gradient: 58-95% MeCN; collection wavelength: 214 nm). The fractions were concentrated under reduced pressure to remove MeCN, and the residue was lyophilized to give 2-((4-(3,3-difluoro-1-(4-(trifluoromethyl)phenyl)cyclobutoxy)-2-methylene-4-oxobutanoyl)oxy)acetic acid (40 mg, 48%) as white solid. LCMS (System 2, Method B) m/z 444.9 (M+Na)⁺ (ES⁺). ¹H NMR (400 MHz, CDCl₃) δ: 7.63 (d, J=8.4 Hz, 2H), 7.52 (d, J=8.0 Hz, 2H), 6.45 (s, 1H), 5.80 (s, 1H), 4.70 (s, 2H), 3.37 (s, 2H), 3.29-3.22 (m, 4H).

### Example 32—3-((2-methylene-4-oxo-4-((3-(4-(trifluoromethyl)phenyl)oxetan-3-yl)oxy)butanoyl)oxy)propanoic acid

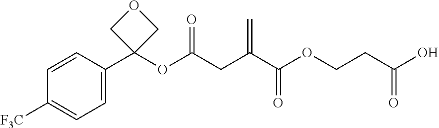 [SMILES: C=C(CC(=O)OC1(c2ccc(C(F)(F)F)cc2)COC1)C(=O)OCCC(=O)O]

Prepared by an analogous method to Example 13 using 2-methylene-4-oxo-4-((3-(4-(trifluoromethyl)phenyl)oxetan-3-yl)oxy)butanoic acid and 2,2,2-trichloroethyl 3-hydroxypropanoate. The crude product was purified by preparative HPLC (Column: Waters Sunfire Prep C18 OBD 10 μm 19×250 mm; Flow Rate: 20 mL/min; solvent system: MeCN/(0.2% formic acid/water); gradient: 55-95% MeCN; collection wavelength: 214 nm). The collected fractions were concentrated under reduced pressure to remove MeCN and the aqueous residue was lyophilized to give 3-((2-methylene-4-oxo-4-((3-(4-(trifluoromethyl)phenyl)oxetan-3-yl)oxy)butanoyl)oxy)propanoic acid (85 mg) as a white solid. LCMS: (System 2, Method B) m/z 424.9 (M+Na)⁺ (ES⁺). ¹H NMR (400 MHz, DMSO-d6) δ: 12.46 (br, 1H), 7.80 (d, J=8.4 Hz, 2H), 7.73 (dd, J=8.4, 1H), 6.21 (d, J=1.2 Hz, 1H), 5.90 (d, J=1.2 Hz, 1H), 4.92 (d, J=8.0 Hz, 2H), 4.83 (d, J=8.4 Hz, 2H), 4.26 (t, J=6.4 Hz, 2H), 3.55 (s, 2H), 2.58 (t, J=6.0 Hz, 2H).

### Example 33—2-(3-((2-methylene-4-oxo-4-(1-(4-(trifluoromethyl)phenyl)cyclobutoxy) butanoyl)oxy)oxetan-3-yl)acetic acid

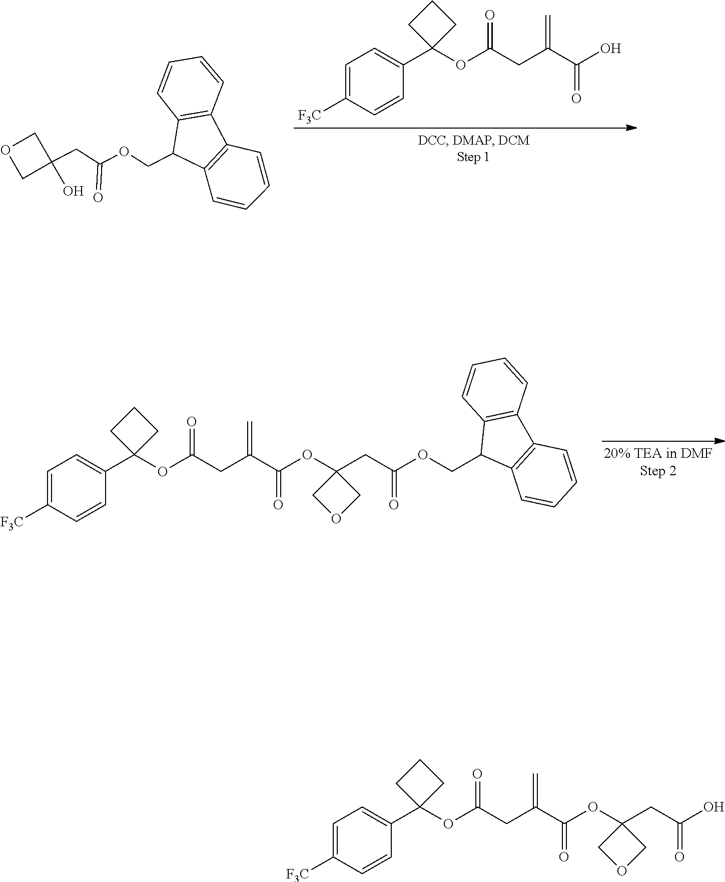 [SMILES: O=C(CC1(O)COC1)OCC1c2ccccc2-c2ccccc21 ; C=C(CC(=O)OC1(c2ccc(C(F)(F)F)cc2)CCC1)C(=O)O ; C=C(CC(=O)OC1(c2ccc(C(F)(F)F)cc2)CCC1)C(=O)OC1(CC(=O)OCC2c3ccccc3-c3ccccc32)COC1 ; C=C(CC(=O)OC1(c2ccc(C(F)(F)F)cc2)CCC1)C(=O)OC1(CC(=O)O)COC1]

Step 1

A mixture of (9H-fluoren-9-yl)methyl 2-(3-hydroxyoxetan-3-yl)acetate (235 mg, 0.76 mmol), 2-methylene-4-oxo-4-(1-(4-(trifluoromethyl)phenyl)cyclobutoxy)butanoic acid (249 mg, 0.76 mmol), DCC (235 mg, 1.14 mmol) and DMAP (12 mg, 0.10 mmol) in DCM (3 mL) was stirred at room temperature for 16 h. The mixture was filtered, and the filtrate was concentrated under reduced pressure. The residue was purified by flash column chromatography (12 g silica, 0-25% MTBE/petroleum ether) to give 1-(3-(2-((9H-fluoren-9-yl)methoxy)-2-oxoethyl)oxetan-3-yl) 4-(1-(4-(trifluoromethyl)phenyl)cyclobutyl) 2-methylenesuccinate (400 mg, 86%) as a colorless oil. LCMS: (System 2, Method C)) m/z 620.8 (M+H)⁺ (ES⁺).

Step 2

A mixture of 1-(3-(2-((9H-fluoren-9-yl)methoxy)-2-oxoethyl)oxetan-3-yl) 4-(1-(4-(trifluoromethyl)phenyl)cyclobutyl) 2-methylenesuccinate (300 mg, 0.48 mmol) in triethylamine (0.2 mL) and DMF (0.8 mL) was stirred at room temperature for 4 h. The reaction mixture was acidified with 0.5N HCl (pH=5), and extracted with EtOAc (3×5 mL). The organic layer was washed by brine, dried over Na₂SO₄ and concentrated under reduced pressure. The residue was purified by prep-HPLC (Column: Waters X-Bridge C18 OBD 10 μm 19×250 mm; Flow Rate: 20 mL/min; solvent system: MeCN/(0.2% formic acid/water) gradient: MeCN: 45-85%; collection wavelength: 214 nm). The fractions were concentrated under reduced pressure to remove MeCN, and the residue was extracted with MTBE (3×5 mL). The combined organic layers were dried over Na₂SO₄ and concentrated under reduced pressure to give 2-(3-((2-methylene-4-oxo-4-(1-(4-(trifluoromethyl)phenyl)cyclobutoxy)butanoyl)oxy)oxetan-3-yl)acetic acid (49 mg, 24%) as colorless oil. LCMS: (System 2, Method B) m/z 442.9 (M+H)⁺ (ES⁺). ¹H NMR (400 MHz, DMSO-d6) δ: 12.50 (br, 1H), 7.75 (d, J=8.4 Hz, 2H), 7.65 (d, J=8.4 Hz, 2H), 6.19 (s, Hz, 1H), 5.87 (s, 1H), 4.65 (d, J=8.0 Hz, 2H), 4.55 (d, J=7.6 Hz, 2H), 3.36 (s, 2H), 2.99 (s, 2H), 2.58-2.55 (m, 4H), 2.01-1.90 (m, 1H), 1.79-1.68 (m, 1H).

### Example 34—2-((2-methylene-4-oxo-4-(1-(4-((trifluoromethyl)thio)phenyl)cyclobutoxy)butanoyl)oxy)acetic acid

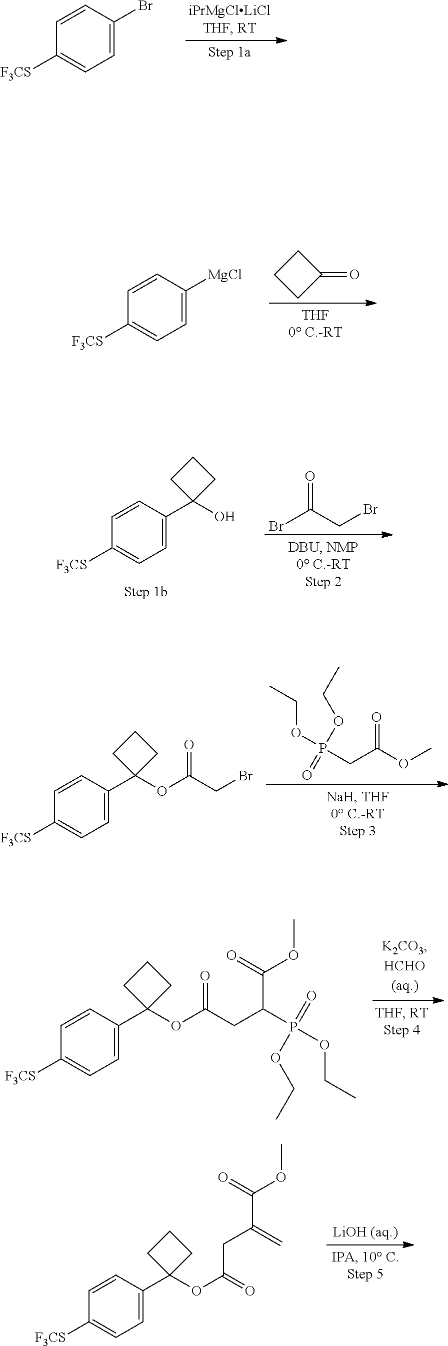

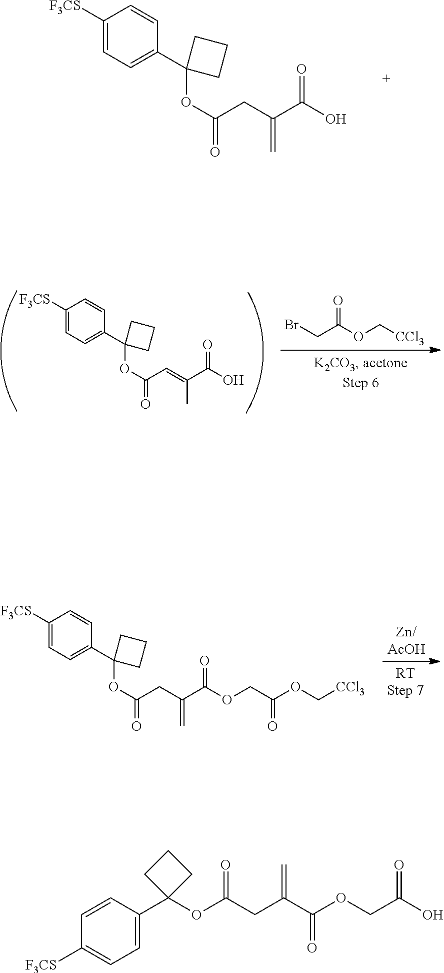

Step 1a

To a solution of (4-bromophenyl)(trifluoromethyl)sulfane (7.75 g, 30 mmol) in THF (75 mL) at room temperature was added a solution of iPrMgCl·LiCl in hexane (1.3 M, 23 ml, 30 mmol) and the mixture was stirred at room temperature for 1 h.

Step 1b

The solution was then cooled to 0° C. and cyclobutanone (1.62 g, 23.2 mmol) was added, and the mixture was stirred at room temperature for 3 h. The reaction mixture was quenched with saturated aqueous NH₄Cl solution (50 mL), the phases were separated and the aqueous layer was extracted with MTBE (2×50 mL). The combined organic layers were washed with brine, dried over Na2SO4, filtered and concentrated under reduced pressure at 30° C. The residue was purified by flash column chromatography (80 g silica, 0-14% MTBE/petroleum ether) to give 1-(4-((trifluoromethyl)thio)phenyl)cyclobutan-1-ol (2.5 g, 33%) as a yellow oil. ¹H NMR (400 MHz, CDCl₃) δ: 7.68-7.63 (m, 2H), 7.59-7.54 (m, 2H), 2.61-2.50 (m, 2H), 2.45-2.33 (m, 2H), 2.16-2.00 (m, 1H), 1.82-1.69 (m, 1H). One exchangable proton not observed.

Step 2

To a solution of 1-(4-((trifluoromethyl)thio)phenyl)cyclobutan-1-ol (2.5 g, 10 mmol) and DBU (3.2 g, 20 mmol) in 1-methyl-2-pyrrolidinone (20 mL) at 0° C. was slowly added 2-bromoacetyl bromide (4.04 g, 20 mmol) dropwise, and the mixture was stirred at room temperature for 16 h. The reaction mixture was diluted with water (20 mL) and MTBE (50 mL), the phases were separated and the aqueous layer was extracted with MTBE (2×30 mL). The combined organic layers were washed with brine, dried over Na₂SO₄, filtered and concentrated under reduced pressure at 40° C. The residue was purified by flash column chromatography (40 g silica, 0-8% MTBE/petroleum ether) to give 1-(4-((trifluoromethyl)thio)phenyl)cyclobutyl 2-bromoacetate (1.5 g, 40%) as a colorless oil. ¹H NMR (400 MHz, CDCl₃) δ: 7.68-7.63 (m, 2H), 7.56-7.51 (m, 2H), 3.76 (s, 2H), 2.75-2.59 (m, 4H), 2.10-1.97 (m, 1H), 1.85-1.70 (m, 1H).

Step 3

To a solution of methyl 2-(diethoxyphosphoryl)acetate (840 mg, 4 mmol) in THF (13 mL) at 0° C. was added NaH suspension in mineral oil (60 wt. %, 160 mg, 4 mmol), and the reaction mixture was stirred at 0° C. for 0.5 h. 1-(4-((Trifluoromethyl)thio)phenyl)cyclobutyl 2-bromoacetate (1.5 g, 4 mmol) was then added and the reaction mixture was stirred at room temperature overnight. The reaction mixture was quenched with dilute aqueous HCl (0.5 M) to pH=5 and the phases were separated. The aqueous layer was extracted with EtOAc (2×20 mL), and the combined organic layers were washed with brine, dried over Na₂SO₄, filtered and concentrated under reduced pressure at 40° C. to give 1-methyl 4-(1-(4-((trifluoromethyl)thio)phenyl)cyclobutyl) 2-(diethoxyphosphoryl)succinate (2.5 g, 98%) as a colorless oil. LCMS: (System 2, Method C) m/z 520.8 (M+Na)⁺ (ES⁺).

Step 4

To a mixture of 1-methyl 4-(1-(4-((trifluoromethyl)thio)phenyl)cyclobutyl) 2-(diethoxyphosphoryl)succinate (2.5 g, 5.0 mmol) and potassium carbonate (1.38 g, 10 mmol) in THF (12 mL) at room temperature was added an aqueous solution of formaldehyde (37 wt. %, 2 mL, 25 mmol), and the reaction mixture was stirred at room temperature for 4 h. The reaction mixture was diluted with H₂O (10 mL), extracted with MTBE (2×20 mL) and the phases were separated. The combined organic layers were washed with H₂O (2×10 mL) and brine, dried over Na₂SO₄, filtered and concentrated under reduced pressure at 30° C. The residue was purified by flash column chromatography (40 g silica, 0-10% MTBE/petroleum ether) to give 1-methyl 4-(1-(4-((trifluoromethyl)thio)phenyl)cyclobutyl) 2-methylenesuccinate (970 mg, 59%) as a colorless oil. LCMS: (System 2, Method C) m/z 396.9 (M+Na)⁺ (ES⁺).

Step 5

To a solution of 1-methyl 4-(1-(4-((trifluoromethyl)thio)phenyl)cyclobutyl) 2-methylenesuccinate (770 mg, 2 mmol) in IPA (7 mL) at 10° C. was added an aqueous solution of LiOH (2 M, 1 mL, 2 mmol), and the reaction mixture was stirred at 10-15° C. for 2 h. The reaction mixture was acidified with dilute aqueous HCl (0.5 M) to pH=3, and extracted with EtOAc (2×20 mL). The combined EtOAc layers were washed with brine, dried over Na₂SO₄, filtered and concentrated under reduced pressure at 30° C. to give a 5:1 mixture of 2-methylene-4-oxo-4-(1-(4-((trifluoromethyl)thio)phenyl)cyclobutoxy)butanoic acid and 2-methyl-4-oxo-4-(1-(4-((trifluoromethyl)thio)phenyl)cyclobutoxy)but-2-enoic acid (670 mg, 70%) as a pale yellow oil, which was used directly in the next step. LCMS: (System 2, Method C) m/z 382.9 (M+Na)⁺ (ES⁺).

Step 6

To a solution of a 5:1 mixture of 2-methylene-4-oxo-4-(1-(4-((trifluoromethyl)thio)phenyl)cyclobutoxy)butanoic acid and 2-methyl-4-oxo-4-(1-(4-((trifluoromethyl)thio)phenyl)cyclobutoxy)but-2-enoic acid (670 mg, 1.8 mmol) in acetone (6 mL) was added potassium carbonate (273 mg, 1.98 mmol) and 2,2,2-trichloroethyl 2-bromoacetate (432 mg, 1.62 mmol) and the reaction mixture was stirred at room temperature overnight. The reaction mixture was filtered, and the filtrate was concentrated under reduced pressure at 30° C. The residue was purified by flash column chromatography (25 g silica, 0-10% MTBE/petroleum ether) to give 1-(2-oxo-2-(2,2,2-trichloroethoxy)ethyl) 4-(1-(4-((trifluoromethyl)thio)phenyl) cyclobutyl) 2-methylenesuccinate (500 mg, 50%) as a pale yellow oil. LCMS: (System 2, Method C) m/z 571.1/572.7 (M+Na)⁺ (ES⁺).

Step 7

To a solution of 1-(2-oxo-2-(2,2,2-trichloroethoxy)ethyl) 4-(1-(4-((trifluoromethyl)thio)phenyl)cyclobutyl) 2-methylenesuccinate (500 mg, 0.9 mmol) in AcOH (5 mL) was added zinc powder (292 mg, 4.5 mmol), and the reaction mixture was stirred at room temperature overnight. The reaction mixture was filtered, and the filtrate was concentrated under reduced pressure at 30° C. The residue was purified by preparative HPLC (Column: Waters X-Bridge C18 OBD 10 μm 19×250 mm; Flow Rate: 20 mL/min; solvent system: MeCN/(0.2% TFA/water); gradient: 58-95% MeCN; collection wavelength: 214 nm). The collected fractions were concentrated under reduced pressure at 30° C. to remove MeCN, and the residue was lyophilized to give 2-((2-methylene-4-oxo-4-(1-(4-((trifluoromethyl)thio)phenyl)cyclobutoxy) butanoyl)oxy)acetic acid (110 mg, 29%) as a colorless oil. LCMS: (System 2, Method B) m/z 440.8 (M+Na)⁺ (ES⁺). ¹H NMR (400 MHz, DMSO-d6) δ: 13.05 (br, 1H), 7.73-7.68 (m, 2H), 7.60-7.54 (m, 2H), 6.27 (d, J=1.2 Hz, 1H), 5.90 (d, J=1.4 Hz, 1H), 4.62 (s, 2H), 3.39 (s, 2H), 2.58-2.50 (m, 4H), 2.02-1.88 (m, 1H), 1.82-1.66 (m, 1H).

### Example 35—3-((2-methylene-4-oxo-4-(1-(4-((trifluoromethyl)thio)phenyl)cyclobutoxy)butanoyl)oxy)propanoic acid

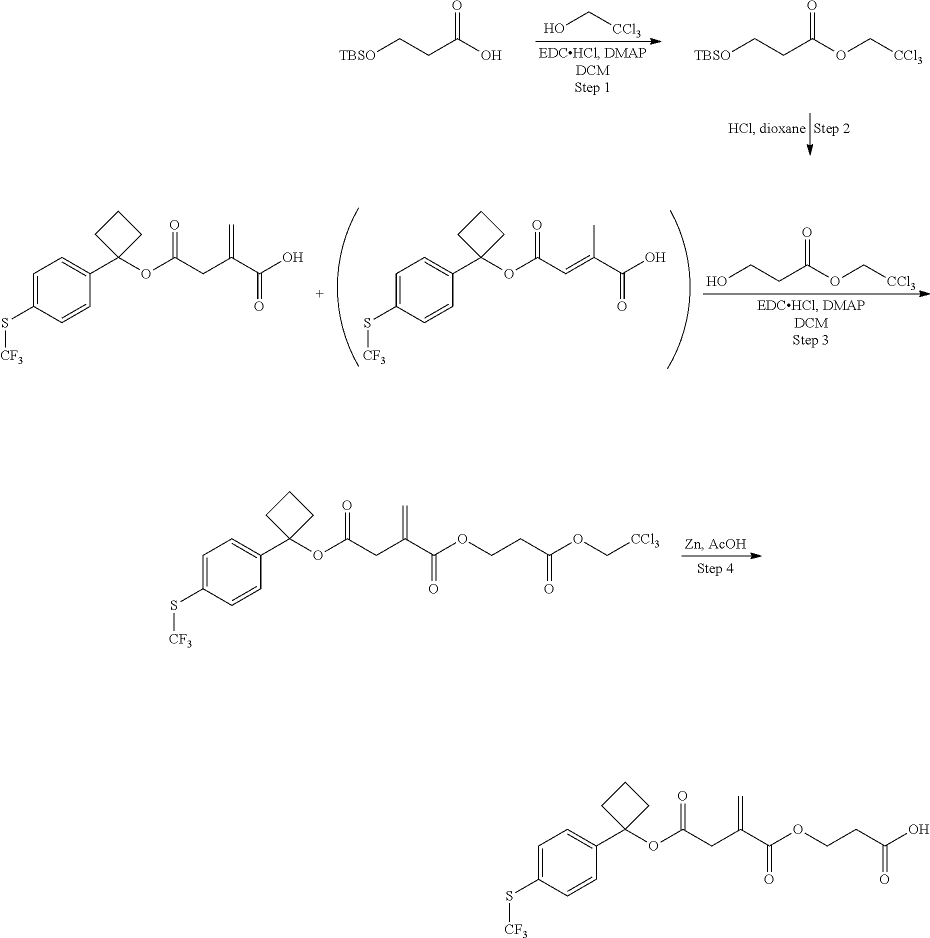

Step 1

To a solution of 3-((tert-butyldimethylsilyl)oxy)propanoic acid, 2,2,2-trichloroethan-1-ol and DMAP in DCM at 0° C. is added EDC·HCl and the resulting mixture is stirred at room temperature for 16 h. The mixture is quenched with saturated aqueous NH₄Cl solution, the phases are separated, and the aqueous phase is extracted with EtOAc. The separated organic phase is washed with brine, dried over Na₂SO₄ and filtered. The filtrate is concentrated under reduced pressure at 35 C, and the residue is purified by flash column chromatography to give 2,2,2-trichloroethyl 3-((tert-butyldimethylsilyl)oxy)propanoate.

Step 2

A solution of 2,2,2-trichloroethyl 3-((tert-butyldimethylsilyl)oxy)propanoate in HCl/dioxane (4 M) is stirred at room temperature for 16 h. The mixture is concentrated under reduced pressure at 30° C. and the residue is purified by flash column chromatography to give 2,2,2-trichloroethyl 3-hydroxypropanoate.

Step 3

To a solution of a mixture of 2-methylene-4-oxo-4-(1-(4-((trifluoromethyl)thio)phenyl)cyclobutoxy)butanoic acid and 2-methyl-4-oxo-4-(1-(4-((trifluoromethyl)thio)phenyl)cyclobutoxy)but-2-enoic acid (Example 1 Step 5), 2,2,2-trichloroethyl 3-hydroxypropanoate, DIPEA and DMAP in DCM at 0° C. is added EDC·HCl, and the resulting mixture is stirred at room temperature for 2 h. The mixture is quenched with dilute aqueous HCl (0.5 M) and adjusted to pH=6, the phases are separated, and the aqueous phase is extracted with DCM. The combined organic layers are washed with brine, dried over Na₂SO₄ and filtered. The filtrate is concentrated under reduced pressure at 30° C., and the residue is purified by flash column chromatography to give 1-(3-oxo-3-(2,2,2-trichloroethoxy)propyl) 4-(1-(4-((trifluoromethyl)thio)phenyl)cyclobutyl) 2-methylenesuccinate.

Step 4

A mixture of 1-(3-oxo-3-(2,2,2-trichloroethoxy)propyl) 4-(1-(4-((trifluoromethyl)thio)phenyl)cyclobutyl) 2-methylenesuccinate and zinc powder in AcOH is stirred at room temperature for 2 days. The mixture is filtered, the filtrate is concentrated under reduced pressure at 20° C. and the residue is purified by preparative HPLC and then further purified by flash column chromatography to give 3-((2-methylene-4-oxo-4-(1-(4-((trifluoromethyl)thio)phenyl)cyclobutoxy)butanoyl)oxy)propanoic acid.

### Biological Example 1—THP-1 AlphaLISA IL-1β and IL-6 Cytokine Assay

Measuring Inhibitory Effects on IL-1β and IL-6 Cytokine Output from THP-1s

The cytokine inhibition profiles of compounds of formula (I) were determined in a differentiated THP-1 cell assay. All assays were performed in RPMI-1640 growth medium (Gibco), supplemented with 10% fetal bovine serum (FBS; Gibco), 1% penicillin-streptomycin and 1% sodium pyruvate unless specified otherwise. The IL-1β and IL-6 cytokine inhibition assays were each run in a background of differentiated THP-1 cells as described below. All reagents described were from Sigma-Aldrich unless specified otherwise. Compounds were prepared as 10 mM DMSO stocks.

Assay Procedure

THP-1 cells were expanded as a suspension up to 80% confluence in appropriate growth medium. Cells were harvested, suspended, and treated with an appropriate concentration of phorbol 12-myristate 13-acetate (PMA) over a 72 hr period (37° C./5% CO₂).

Following 72 hrs of THP-1 cell incubation, cellular medium was removed and replaced with fresh growth media containing 1% of FBS. Working concentrations of compounds were prepared separately in 10% FBS treated growth medium and pre-incubated with the cells for 30 minutes (37° C./5% CO₂). Following the 30 minute compound pre-incubation, THP-1s were treated with an appropriate concentration of LPS and the THP-1s were subsequently incubated for a 24 hr period (37° C./5% CO₂). An appropriate final concentration of Nigericin was then dispensed into the THP-1 plates and incubated for 1 hour (37° C./5% CO₂) before THP-1 supernatants were harvested and collected in separate polypropylene 96-well holding plates.

Reagents from each of the IL-1L and IL-6 commercial kits (Perkin Elmer) were prepared and run according to the manufacturer's instructions. Subsequently, fluorescence signal detection in a microplate reader was measured (EnVision® Multilabel Reader, Perkin Elmer).

Percentage inhibition was calculated per cytokine by normalising the sample data to the high and low controls used within each plate (+/−LPS respectively). Percentage inhibition was then plotted against compound concentration and the 50% inhibitory concentration (IC₅₀) was determined from the resultant concentration-response curve.

Compounds of formula (I) were tested and the results are shown in Table 1 below. Dimethyl itaconate was included as a comparator compound.

TABLE 1
THP-1 cell IL-1β and IL-6 IC₅₀ values (μM)

| Compound | IL-1β (IC₅₀) | IL-6 (IC₅₀) |
|---|---|---|
| dimethyl itaconate | >100 | >100.0 |
| Example 1 | 9.4 | 4.6 |
| Example 2 | 7.7 | 5.2 |
| | 9.1^{a} | 7.7^{a} |
| Example 3 | 12.1 | 11.3 |
| Example 4 | 12.4 | 6.9 |
| Example 5 | 10.6 | 5.3 |
| Example 6 | 12.7 | NT* |
| Example 7 | 17.4 | NT |
| Example 8 | 15.2 | 6.0 |
| Example 9 | 12.1 | NT |
| Example 10 | 11.3 | NT |
| Example 11 | 9.0 | NT |
| Example 12 | 8.8 | NT |
| Example 13 | 12.5 | 5.9 |
| Example 14 | 19.9 | NT |
| Example 15 | 8.6 | NT |
| Example 16 | 4.9 | NT |
| Example 17 | 4.9 | NT |
| dimethyl itaconate | >100 | >100.0 |
| Example 18 | 9.9 | NT |
| Example 19 | 4.4 | NT |
| Example 20 | 8.7 | NT |
| Example 21 | 1.0 | 0.9 |
| Example 22 | 10.3 | 11.2 |
| Example 23 | 1.5 | 1.2 |
| Example 24 | 14.5 | 11.3 |
| Example 25 | 17.4 | 15.9 |
| Example 26 | 46.9 | 3.9 |
| Example 27 | 16.4 | 14.1 |
| Example 28 | 11.3 | 3.3 |
| Example 29 | 43.2 | 45.2 |
| Example 30 | 16.7 | >100 |
| Example 31 | 14.4 | 16.5 |
| Example 32 | 7.4 | 11.9 |
| Example 33 | 2.4 | 5.7 |
| Example 34 | 5.1 | 2.7 |

NT* means not tested in this assay;
^{a}from repeated experiments

All compounds of formula (I) shown in Table 1 exhibited improved cytokine-lowering potencies compared to dimethyl itaconate for IL-1β and/or IL-6 (where tested).

### Biological Example 2—NRF2+/−GSH Activation Assay

Measuring Compound Activation Effects on the Anti-Inflammatory Transcription Factor NRF2 in DiscoverX PathHunter NRF2 Translocation Kit

Potency and efficacy of compounds of formula (I) against the target of interest to activate NRF2 (nuclear factor erythroid 2-related factor 2) were determined using the PathHunter NRF2 translocation kit (DiscoverX). The NRF2 translocation assay was run using an engineered recombinant cell line, utilising enzyme fragment complementation to determine activation of the Keap1-NRF2 protein complex and subsequent translocation of NRF2 into the nucleus. Enzyme activity was quantified using a chemiluminescent substrate consumed following the formation of a functional enzyme upon PK-tagged NRF2 translocation into the nucleus.

The assay was run under either +/−GSH (glutathione) conditions to determine the attenuating activities of GSH against target compounds.

Additionally, a defined concentration of dimethyl fumarate was used as the ‘High’ control to normalise test compound activation responses to.

Assay Procedure

U2OS PathHunter eXpress cells were thawed from frozen prior to plating. Following plating, U2OS cells were incubated for 24 hrs (37° C./5% CO₂) in commercial kit provided cell medium.

Following 24 hrs of U2OS incubation, cells were directly treated with an appropriate final concentration of compound, for −GSH conditions, or for +GSH conditions, an intermediate plate containing 6× working concentrations of compound stocks was prepared in a 6 mM working concentration of GSH solution (solubilised in sterile PBS). Following a 30 minute compound-GSH pre-incubation (37° C./5% CO₂) for +GSH treatment, plated U2OS cells were incubated with an appropriate final concentration of compound and GSH.

Following compound (+/−GSH) treatment, the U2OS plates were incubated for a further 6 hours (37° C./5% CO₂) before detection reagent from the PathHunter NRF2 commercial kit was prepared and added to test plates according to the manufacturer's instructions. Subsequently, the luminescence signal detection in a microplate reader was measured (PHERAstar®, BMG Labtech).

Percentage activation was calculated by normalising the sample data to the high and low controls used within each plate (+/−DMF). Percentage activation/response was then plotted against compound concentration and the 50% activation concentration (EC₅₀) was determined from the plotted concentration-response curve.

A number of compounds of formula (I) were tested, and the results are shown in Table 2 below. Dimethyl itaconate and dimethyl fumarate were included as a comparator compounds.

TABLE 2
NRF2 activation

| | −GSH | | +GSH | |
|---|---|---|---|---|
| Compound | EC₅₀ (μM) | Eₘₐₓ (%) | EC₅₀ (μM) | Eₘₐₓ (%) |
| dimethyl fumarate | 6.0 | 100 | >100 | 19 |
| dimethyl itaconate | 21.4 | 137 | 56.4 | 77 |
| Example 1 | 7.7 | 375 | 11.1 | 259 |
| Example 2 | 15.4 | 217 | 21.6 | 167 |
| Example 4 | 19.0 | 192 | 28.9 | 171 |
| Example 5 | 16.0 | 208 | 25.4 | 185 |
| Example 6 | 11.9 | 219 | 15.4 | 187 |
| Example 8 | 12.2 | 193 | 18.7 | 170 |
| Example 9 | 5.1 | 210 | 7.3 | 182 |
| Example 10 | 8.3 | 236 | 11.6 | 175 |
| Example 11 | 3.0 | 214 | 5.1 | 160 |
| Example 12 | 5.2 | 217 | 7.9 | 158 |
| Example 13 | 7.6 | 251 | 11.6 | 143 |
| Example 21 | 1.7 | 170 | 5.7 | 53 |
| Example 22 | 14.9 | 225 | 22.3 | 183 |
| Example 23 | 2.3 | 141 | 5.3 | 54 |
| Example 24 | 29.6 | 194 | 28.3 | 119 |
| Example 31 | 21.7 | 182 | 30.8 | 108 |
| Example 33 | 8.4 | 162 | 20.7 | 118 |
| Example 34 | >54.8 | 0 | >100 | 2.8 |

All compounds of formula (I) that are shown in Table 2, except Example 34, exhibited a lower EC₅₀ and/or a higher Eₘₐₓ compared with dimethyl itaconate in both −GSH and +GSH assays. All compounds of formula (I) that are shown in Table 2, except Example 34, exhibited a lower EC₅₀ and a higher Eₘₐₓ compared with dimethyl fumarate in the +GSH assay, i.e., unlike dimethyl fumarate, the NRF2-activating properties of the compounds of formula (I) are largely retained in the presence of glutathione (GSH). Certain compounds of formula (I) that are shown in Table 2 exhibited a lower EC₅₀ and/or higher Eₘₐₓ compared with dimethyl fumarate in the −GSH assay.

### Biological Example 3—Hepatocyte Stability Assay

Defrosted cryo-preserved hepatocytes (viability>70%) may be used to determine the metabolic stability of a compound via calculation of intrinsic clearance (Clᵢₙₜ; a measure of the removal of a compound from the liver in the absence of blood flow and cell binding). Clearance data are particularly important for in vitro work as they can be used in combination with in vivo data to predict the half-life and oral bioavailability of a drug.

The metabolic stability in hepatocytes assay involves a time-dependent reaction using both positive and negative controls. The cells were pre-incubated at 37° C. and spiked with test compound (and positive control); samples were taken at pre-determined time intervals and analysed to monitor the change in concentration of the initial drug compound over 60 minutes. A buffer incubation reaction (with no hepatocytes present) acted as a negative control and two cocktail solutions, containing compounds with known high and low clearance values (verapamil/7-hydroxycoumarin and propranolol/diltiazem), acted as positive controls.
1. The assay was run with a cell concentration of 0.5×10⁶ cells/mL in Leibovitz buffer.
2. All compounds and controls were run in duplicate.
3. Compound concentration was 10 μM.
4. All compounds and controls were incubated with both cells and buffer to show turnover was due to hepatic metabolism.
5. All wells on the incubation plate had 326.7 μL of either cells or buffer added.
6. Prior to assay, cell and buffer-only incubation plates were preincubated for 10 mins at 37° C.
7. The assay was initiated by adding compounds, 3.3 μL of 1 mM in 10% DMSO-90% Buffer; final DMSO concentration is 0.1%.
8. Samples were taken at regular timepoints (0, 5, 10, 20, 40, 60 min) until 60 mins.
9. Sample volume was 40 μL and was added to 160 μL of crash solvent (acetonitrile with internal standard) and stored on ice.
10. At the end of the assay, the crash plates were centrifuged at 3500 rpm for 20 mins at 4° C.
11. 80 μL of clear supernatant was removed and mixed with 80 μL of deionised water before being analysed by LC-MS/MS.

Raw LC-MS/MS data was exported to, and analysed in, Microsoft Excel for determination of intrinsic clearance. The percentage remaining of a compound was monitored using the peak area of the initial concentration as 100%. Intrinsic clearance and half-life values were calculated using a graph of the natural log of percentage remaining versus the time of reaction in minutes. Half-life (min) and intrinsic clearance (Clᵢₙₜ in μL min⁻¹ 10⁻⁶ cells) values were calculated using the gradient of the graph (the elimination rate constant, k) and Equations 1 and 2.

$${t}_{\frac{1}{2}} = \frac{ln 2}{k} \qquad \{ Equation 1 \}$$

$${Cl}_{i n t} = ( \frac{ln 2}{{t}_{\frac{1}{2}}} ) \times ( \frac{350}{0.175} ) \qquad \{ Equation 2 \}$$

A number of compounds of formula (I) were tested in this assay, and the results are shown in Table 3 below. 4-Octyl itaconate was included as a comparator compound.

TABLE 3
Hepatocyte stability

| Compound | Species | CIᵢₙₜ (μL min⁻¹ 10⁻⁶ cells) | T½ (min) |
|---|---|---|---|
| 4-octyl itaconate | Human | 401 | 4 |
| | Mouse | 351 | 4 |
| Example 1 | Human | 13 | 116 |
| | Mouse | 52 | 24 |
| Example 2 | Human | <3 | >460 |
| | Mouse | 50 | 38 |
| Example 3 | Human | 9 | 174 |
| | Mouse | 95 | 20 |
| Example 4 | Human | 8 | 189 |
| | Mouse | 69 | 28 |
| Example 5 | Human | 9 | 172 |
| | Mouse | 80 | 24 |
| Example 6 | Human | 18 | 85 |
| | Mouse | >460 | <3 |
| Example 7 | Human | 10 | 151 |
| | Mouse | 107 | 18 |
| Example 8 | Human | 11 | 142 |
| | Mouse | 223 | 9 |
| Example 9 | Human | 6 | 251 |
| | Mouse | 66 | 29 |
| Example 10 | Human | 14 | 107 |
| | Mouse | 187 | 8 |
| Example 11 | Human | 10 | 152 |
| | Mouse | 100 | 15 |
| 4-octyl itaconate | Human | 401 | 4 |
| | Mouse | 351 | 4 |
| Example 12 | Human | 23 | 64 |
| | Mouse | 239 | 6 |
| Example 13 | Human | 16 | 100 |
| | Mouse | >460 | <3 |
| Example 16 | Human | 7 | 209 |
| | Mouse | 61 | 25 |
| Example 17 | Human | 8 | 188 |
| | Mouse | 73 | 21 |
| Example 18 | Human | 13 | 109 |
| | Mouse | 58 | 26 |
| Example 19 | Human | 8 | 187 |
| | Mouse | 66 | 23 |
| Example 20 | Human | 15 | 84 |
| | Mouse | 77 | 26 |
| Example 22 | Human | 9 | 156 |
| | Mouse | 128 | 12 |
| Example 30 | Human | 16 | 86 |
| | Mouse | 33 | 39 |
| Example 32 | Human | 4 | 407 |
| | Mouse | 36 | 41 |
| Example 33 | Human | 7 | 202 |
| | Mouse | 82 | 18 |
| Example 34 | Human | 11 | 116 |
| | Mouse | 74 | 23 |

All compounds of formula (I) that are shown in Table 3 have improved metabolic stabilities compared with 4-octyl itaconate, as shown by their intrinsic clearance (Clᵢₙₜ) and half-life (T_{1/2}) values in this assay. All the compounds of formula (I) shown in Table 3 were more stable, i.e., they exhibited lower intrinsic clearance (Clᵢₙₜ) and longer half-life (T_{1/2}) values compared with 4-octyl itaconate in at least human or mouse species. Preferred compounds exhibited much lower intrinsic clearance (Clᵢₙₜ) and much longer half-life (T_{1/2}) values compared with 4-octyl itaconate in both human and mouse species.

### REFERENCES

The following publication cited in this specification are herein incorporated by reference in their entirety.
Ackermann et al. Proc. Soc. Exp. Bio. Med. 1949, 72(1), 1-9.
Andersen J. L. et al. Nat. Commun. 2018, 9, 4344.
Angiari S. and O'Neill L. A. Cell Res. 2018, 28, 613-615.
Bagavant G. et al. Indian J. Pharm. Sci. 1994, 56, 80-85.
Bambouskova M. et al. Nature 2018, 556, 501-504.
Blewett M. M. et al. Sci. Sign. 2016, 9 (445), rs10; 6.
Brennan M. S. et al. PLoS One 2015, 10, e0120254.
Brück J. et al. Exp. Dermatol. 2018, 27, 611-624.
Cocco M. et al. J. Med. Chem. 2014, 57, 10366-10382.
Cocco M. et al. J. Med. Chem. 2017, 60, 3656-3671.
Cordes T. et al. J. Biol. Chem. 2016, 291, 14274-14284.
Cordes T. et al. Mol. Metab. 2020, 32, 122-135.
Daly R. et al. medRxiv 2019, 19001594; doi: https://doi.org/10.1101/19001594.
Daniels B. P. et al. Immunity 2019, 50(1), 64-76.e4.
Dibbert S. et al. Arch. Dermatol. Res. 2013, 305, 447-451.
ElAzzouny M. et al. J. Biol. Chem. 2017, 292, 4766-4769.
Gillard G. O. et al. J. Neuroimmunol. 2015, 283, 74-85.
Gu L. et al. Immunol. Cell Biol. 2020 doi:10.1111/imcb.12316.
Hanke T. et al. Pharmacol. Therapeut. 2016, 157, 163-187.
Hunt T. et al. Consortium of Multiple Sclerosis Centers 2015 Annual Meeting, 27-30 May 2015, Indianapolis, IN, USA: Poster DX37.
Kornberg M. D. et al. Science 2018, 360, 449-453.
Kulkarni R. A. et al. Nat. Chem. Biol. 2019 10.1038/s41589-018-0217-y.
Lampropoulou V. et al. Cell Metab. 2016, 24, 158-166.
Lehmann J. C. U. et al. J. Invest. Dermatol. 2007, 127, 835-845.
Liao S.-T. et al. Nat. Commun. 2019, 10(1), 5091.
Liu H. et al. Cell Commun. Signal. 2018, 16, 81.
McGuire V. A. et al. Sci. Rep. 2016, 6, 31159.
Michelucci A. et al. Proc. Natl. Acad. Sci. USA 2013, 110, 7820-7825.
Mills E. A. et al. Front. Neurol. 2018, 9, 5.
Mills E. L. et al. Cell 2016, 167, 457-470.
Mills E. L. et al. Nature 2018, 556, 113-117.
Mrowietz U. et al. Trends Pharmacol. Sci. 2018, 39, 1-12.
Müller S. et al. J. Dermatol. Sci. 2017, 87, 246-251.
Murphy M. P. and O'Neill L. A. J. Cell 2018, 174, 780-784.
O'Neill L. A. J. and Artyomov M. N. Nat. Rev. Immunol. 2019 273-281.
Olagnier D. et al. Nat. Commun. 2018, 9, 3506.
Schmidt T. J. et al. Bioorg. Med. Chem. 2007, 15, 333-342.
Shan Q. et al. Biochem. Biophys. Res. Commun. 2019, 517, 538-544.
Straub R. H. and Schradin C. Evol. Med. Public Health 2016, 1, 37-51S.
Straub R. H. and Cutolo M. Rheumatology 2016, 55 (Suppl. 2), ii6-ii14.
Sun X. et al., FASEB J. 2019, 33, 12929-12940.
Tang C. et al. Cell Physiol. Biochem. 2018, 51, 979-990.
Tang C. et al. Biochem. Biophys. Res. Commun. 2019, 508, 921-927.
Tang H. et al. Biochem. Biophys. Res. Commun. 2008, 375, 562-565.
Tian et al. Eur. J. Pharmacol. 2020, 873, 172989.
van der Reest J. et al. Nat. Commun. 2018, 9, 1581.
von Glehn F. et al. Mult. Scler. Relat. Disord. 2018, 23, 46-50.
Yi F. et al. Hepatology 2020, 873, 172989.
Yu X.-H. et al. Immunol. Cell Biol. 2019, 97, 134-141.
Zhang S. et al. Bioorg. Med. Chem. 2012, 20, 6073-6079.
Zhang D. et al. Int. Immunopharmacol. 2019, 77, 105924.
Zhao C. et al. Microb. Pathogen. 2019, 133, 103541.
Zhao G. et al. Biochem. Biophys. Res. Commun. 2014, 448, 303-307.

Miscellaneous

All references referred to in this application, including patent and patent applications, are incorporated herein by reference to the fullest extent possible.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims.

1. A compound of formula (I): 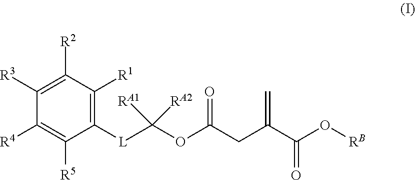
wherein:
R^{A1} and R^{A2} join to form a C₃₋₅ cycloalkyl ring: 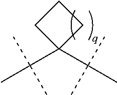
wherein q is 0, 1 or 2; or
R^{A1} and R^{A2} join to form a 4-6-membered heterocyclic ring;
wherein the C₃₋₅ cycloalkyl ring and 4-6-membered heterocyclic ring are optionally substituted by one or more groups selected from methyl and fluoro;
R¹ is H or Cl;
R² is H or halo;
R³ is H, halo, CH₃, OCH₃, CF₃, OCF₃, SC₁₋₄alkyl, SC₁₋₄haloalkyl, or phenyl which is optionally substituted by halo, or —C≡C—C₁₋₂haloalkyl;
R⁴ is H, halo or CF₃;
R⁵ is H or Cl;
wherein one, two or three of any of the R¹ to R⁵ groups are other than H and the remainder are H;
L is a bond, C₁₋₂alkylene or C≡C; and
R^{B} is selected from the group consisting of CH₂COOH, CH₂CH₂COOH, CH₂tetrazolyl and CH₂CH₂tetrazolyl wherein R^{B} is optionally substituted on an available carbon atom by one or more R^{B1} wherein R^{B1} is selected from the group consisting of difluoromethyl, trifluoromethyl and methyl, and/or two R^{B1} groups which are attached to the same carbon atom join to form a C₃₋₆ cycloalkyl ring or a 4-6-membered heterocyclic ring;
or a pharmaceutically acceptable salt and/or solvate thereof.

2-5. (canceled)

6. The compound or a pharmaceutically acceptable salt and/or solvate thereof according to claim 1 wherein R^{A1} and R^{A2} join to form a C₃₋₅ cycloalkyl ring: 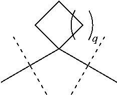
wherein q is 0, 1 or 2.

7-9. (canceled)

10. The compound or a pharmaceutically acceptable salt and/or solvate thereof according to claim 1 wherein R^{A1} and R^{A2} join to form a 4-6-membered heterocyclic ring.

11-13. (canceled)

14. The compound or a pharmaceutically acceptable salt and/or solvate thereof according to claim 1 wherein the C₃₋₅ cycloalkyl ring or 4-6-membered heterocyclic ring is not substituted.

15-18. (canceled)

19. The compound or a pharmaceutically acceptable salt and/or solvate thereof according to claim 1 wherein R¹ is H; or wherein R¹ is Cl.

20-21. (canceled)

22. The compound or a pharmaceutically acceptable salt and/or solvate thereof according to claim 1 wherein R² is halo.

23. (canceled)

24. The compound or a pharmaceutically acceptable salt and/or solvate thereof according to claim 1 wherein R³ is halo; or wherein R³ is CF₃; or wherein R³ is SC₁₋₄haloalkyl.

25-40. (canceled)

41. The compound or a pharmaceutically acceptable salt and/or solvate thereof according to claim 1 wherein L is a bond.

42-43. (canceled)

44. The compound or a pharmaceutically acceptable salt and/or solvate thereof according to claim 1 wherein R^{B} is CH₂COOH optionally substituted on an available carbon atom by one or more R^{B1}; or wherein R^{B} is CH₂CH₂COOH optionally substituted on an available carbon atom by one or more R^{B1}.

45-47. (canceled)

48. The compound or a pharmaceutically acceptable salt and/or solvate thereof according to claim 1 wherein R^{B} is not substituted.

49. The compound or a pharmaceutically acceptable salt and/or solvate thereof according to claim 1 wherein R^{B} is substituted by one or more R^{B1}, wherein R^{B1} is trifluoromethyl.

50-53. (canceled)

54. The compound or a pharmaceutically acceptable salt and/or solvate thereof according to claim 1 wherein two R^{B1} groups which are attached to the same carbon atom join to form a 4-6-membered heterocyclic ring.

55-58. (canceled)

59. The compound according to claim 1 which is selected from the list consisting of:
2-((4-(1-(3,5-dichlorophenyl)cyclobutoxy)-2-methylene-4-oxobutanoyl)oxy)acetic acid;
2-((2-methylene-4-oxo-4-(1-(4-(trifluoromethyl)phenyl)cyclobutoxy) butanoyl)oxy)acetic acid;
2-((4-(1-(4-bromophenyl)cyclobutoxy)-2-methylene-4-oxobutanoyl)oxy)acetic acid;
2-((4-(1-(4-chlorophenyl)cyclobutoxy)-2-methylene-4-oxobutanoyl)oxy)acetic acid;
2-((4-(1-(2,5-dichlorophenyl)cyclobutoxy)-2-methylene-4-oxobutanoyl)oxy)acetic acid;
2-((4-(1-(2-chloro-4-(trifluoromethoxy)phenyl)cyclobutoxy)-2-methylene-4-oxobutanoyl)oxy)acetic acid;
2-((4-(1-(2,3-dichlorophenyl)cyclobutoxy)-2-methylene-4-oxobutanoyl)oxy)acetic acid;
2-((4-(1-(2,6-dichlorophenyl)cyclobutoxy)-2-methylene-4-oxobutanoyl)oxy)acetic acid;
2-((2-methylene-4-oxo-4-(1-(4-(trifluoromethoxy)phenyl)cyclobutoxy) butanoyl)oxy)acetic acid;
2-((4-(1-(2,4-dichlorophenyl)cyclobutoxy)-2-methylene-4-oxobutanoyl)oxy)acetic acid;
2-((4-(1-(2-chloro-4-(trifluoromethyl)phenyl)cyclobutoxy)-2-methylene-4-oxobutanoyl)oxy)acetic acid;
2-((4-(1-(4-bromo-2-chlorophenyl)cyclobutoxy)-2-methylene-4-oxobutanoyl)oxy)acetic acid;
3-((4-(1-(3,5-dichlorophenyl)cyclobutoxy)-2-methylene-4-oxobutanoyl)oxy)propanoic acid;
2-((2-methylene-4-oxo-4-(1-(3,4,5-trifluorophenyl)cyclobutoxy)butanoyl)oxy) acetic acid;
2-((4-(1-(3-chloro-5-(trifluoromethyl)phenyl)cyclobutoxy)-2-methylene-4-oxobutanoyl)oxy)acetic acid;
2-((4-(1-(3-chloro-4-(trifluoromethyl)phenyl)cyclobutoxy)-2-methylene-4-oxobutanoyl)oxy)acetic acid;
2-((4-(1-(3,5-dichloro-4-fluorophenyl)cyclobutoxy)-2-methylene-4-oxobutanoyl)oxy)acetic acid;
2-((4-(1-(3,5-dichloro-4-methoxyphenyl)cyclobutoxy)-2-methylene-4-oxobutanoyl)oxy)acetic acid;
2-((4-(1-(3,5-difluoro-4-(trifluoromethyl)phenyl)cyclobutoxy)-2-methylene-4-oxobutanoyl)oxy)acetic acid;
2-((4-(1-(3-chloro-4-methylphenyl)cyclobutoxy)-2-methylene-4-oxobutanoyl)oxy)acetic acid;
(S)-4,4,4-trifluoro-3-((2-methylene-4-oxo-4-(1-(4-(trifluoromethyl)phenyl)cyclobutoxy)butanoyl)oxy)butanoic acid;
3-((2-methylene-4-oxo-4-(1-(4-(trifluoromethyl)phenyl)cyclobutoxy)butanoyl)oxy)propanoic acid;
(R)-4,4,4-trifluoro-3-((2-methylene-4-oxo-4-(1-(4-(trifluoromethyl)phenyl)cyclobutoxy)butanoyl)oxy)butanoic acid;
2-((4-(1-(4-chlorobenzyl)cyclobutoxy)-2-methylene-4-oxobutanoyl)oxy)acetic acid;
2-((2-methylene-4-oxo-4-((1-(4-(trifluoromethyl)phenyl)cyclopentyl)oxy)butanoyl)oxy)acetic acid;
2-((4-(1-(4′-fluoro-[1,1′-biphenyl]-4-yl)cyclobutoxy)-2-methylene-4-oxobutanoyl)oxy)acetic acid;
2-((2-methylene-4-oxo-4-(1-(4-(trifluoromethyl)benzyl)cyclopropoxy)butanoyl)oxy)acetic acid;
2-((2-methylene-4-oxo-4-(1-((4-(trifluoromethyl)phenyl)ethynyl)cyclobutoxy)butanoyl)oxy)acetic acid;
2-((2-methylene-4-oxo-4-((3-(4-(trifluoromethyl)phenyl)oxetan-3-yl)oxy)butanoyl)oxy)acetic acid;
2-((2-methylene-4-oxo-4-(1-(4-(trifluoromethyl)phenyl) cyclopropoxy)butanoyl)oxy)acetic acid;
2-((4-(3,3-difluoro-1-(4-(trifluoromethyl)phenyl)cyclobutoxy)-2-methylene-4-oxobutanoyl)oxy)acetic acid;
3-((2-methylene-4-oxo-4-((3-(4-(trifluoromethyl)phenyl)oxetan-3-yl)oxy)butanoyl)oxy)propanoic acid;
2-(3-((2-methylene-4-oxo-4-(1-(4-(trifluoromethyl)phenyl)cyclobutoxy) butanoyl)oxy)oxetan-3-yl)acetic acid; and
2-((2-methylene-4-oxo-4-(1-(4-((trifluoromethyl)thio)phenyl) cyclobutoxy)butanoyl)oxy)acetic acid;
or a pharmaceutically acceptable salt and/or solvate of any one thereof.

60. A pharmaceutical composition comprising a compound or a pharmaceutically acceptable salt and/or solvate thereof according to claim 1.

61-63. (canceled)

64. A method of treating or preventing an inflammatory disease or a disease associated with an undesirable immune response, which comprises administering a compound or a pharmaceutically acceptable salt and/or solvate thereof according to claim 1.

65-68. (canceled)

69. The method according to claim 64, wherein the inflammatory disease or disease associated with an undesirable immune response is, or is associated with, a disease selected from the group consisting of: psoriasis (including chronic plaque, erythrodermic, pustular, guttate, inverse and nail variants), asthma, chronic obstructive pulmonary disease (COPD, including chronic bronchitis and emphysema), heart failure (including left ventricular failure), myocardial infarction, angina pectoris, other atherosclerosis and/or atherothrombosis-related disorders (including peripheral vascular disease and ischaemic stroke), a mitochondrial and neurodegenerative disease autoimmune paraneoplastic retinopathy, transplantation rejection (including antibody-mediated and T cell-mediated forms), multiple sclerosis, transverse myelitis, ischaemia-reperfusion injury, AGE-induced genome damage, an inflammatory bowel disease, primary sclerosing cholangitis (PSC), PSC-autoimmune hepatitis overlap syndrome, non-alcoholic fatty liver disease (non-alcoholic steatohepatitis), rheumatica, granuloma annulare, cutaneous lupus erythematosus (CLE), systemic lupus erythematosus (SLE), lupus nephritis, drug-induced lupus, autoimmune myocarditis or myopericarditis, Dressler's syndrome, giant cell myocarditis, post-pericardiotomy syndrome, drug-induced hypersensitivity syndromes (including hypersensitivity myocarditis), eczema, sarcoidosis, erythema nodosum, acute disseminated encephalomyelitis (ADEM), neuromyelitis optica spectrum disorders, MOG (myelin oligodendrocyte glycoprotein) antibody-associated disorders (including MOG-EM), optic neuritis, CLIPPERS (chronic lymphocytic inflammation with pontine perivascular enhancement responsive to steroids), diffuse myelinoclastic sclerosis, Addison's disease, alopecia areata, ankylosing spondylitis, other spondyloarthritides (including peripheral spondyloarthritis, that is associated with psoriasis, inflammatory bowel disease, reactive arthritis or juvenile onset forms), antiphospholipid antibody syndrome, autoimmune hemolytic anaemia, autoimmune hepatitis, autoimmune inner ear disease, pemphigoid (including bullous pemphigoid, mucous membrane pemphigoid, cicatricial pemphigoid, herpes gestationis or pemphigoid gestationis, ocular cicatricial pemphigoid), linear IgA disease, Behçet's disease, celiac disease, Chagas disease, dermatomyositis, diabetes mellitus type I, endometriosis, Goodpasture's syndrome, Graves' disease, Guillain-Barre syndrome and its subtypes (including acute inflammatory demyelinating polyneuropathy, AIDP, acute motor axonal neuropathy (AMAN), acute motor and sensory axonal neuropathy (AMSAN), pharyngeal-cervical-brachial variant, Miller-Fisher variant and Bickerstaff s brainstem encephalitis), progressive inflammatory neuropathy, Hashimoto's disease, hidradenitis suppurativa, inclusion body myositis, necrotising myopathy, Kawasaki disease, IgA nephropathy, Henoch-Schonlein purpura, idiopathic thrombocytopenic purpura, thrombotic thrombocytopenic purpura (TTP), Evans' syndrome, interstitial cystitis, mixed connective tissue disease, undifferentiated connective tissue disease, morphea, myasthenia gravis (including MuSK antibody positive and seronegative variants), narcolepsy, neuromyotonia, pemphigus vulgaris, pernicious anaemia, psoriatic arthritis, polymyositis, primary biliary cholangitis (also known as primary biliary cirrhosis), rheumatoid arthritis, palindromic rheumatism, schizophrenia, autoimmune (meningo-)encephalitis syndromes, scleroderma, Sjogren's syndrome, stiff person syndrome, polymylagia rheumatica, giant cell arteritis (temporal arteritis), Takayasu arteritis, polyarteritis nodosa, Kawasaki disease, granulomatosis with polyangitis (GPA; formerly known as Wegener's granulomatosis), eosinophilic granulomatosis with polyangiitis (EGPA; formerly known as Churg-Strauss syndrome), microscopic polyarteritis/polyangiitis, hypocomplementaemic urticarial vasculitis, hypersensitivity vasculitis, cryoglobulinemia, thromboangiitis obliterans (Buerger's disease), vasculitis, leukocytoclastic vasculitis, vitiligo, acute disseminated encephalomyelitis, adrenoleukodystrophy, Alexander's disease, Alper's disease, balo concentric sclerosis or Marburg disease, cryptogenic organising pneumonia (formerly known as bronchiolitis obliterans organizing pneumonia), Canavan disease, central nervous system vasculitic syndrome, Charcot-Marie-Tooth disease, childhood ataxia with central nervous system hypomyelination, chronic inflammatory demyelinating polyneuropathy (CIDP), diabetic retinopathy, globoid cell leukodystrophy (Krabbe disease), graft-versus-host disease (GVHD) (including acute and chronic forms, as well as intestinal GVHD), hepatitis C (HCV) infection or complication, herpes simplex viral infection or complication, human immunodeficiency virus (HIV) infection or complication, lichen planus, monomelic amyotrophy, cystic fibrosis, pulmonary arterial hypertension (PAH, including idiopathic PAH), lung sarcoidosis, idiopathic pulmonary fibrosis, paediatric asthma, atopic dermatitis, allergic dermatitis, contact dermatitis, allergic rhinitis, rhinitis, sinusitis, conjunctivitis, allergic conjunctivitis, keratoconjunctivitis sicca, dry eye, xerophthalmia, glaucoma, macular oedema, diabetic macular oedema, central retinal vein occlusion (CRVO), macular degeneration (including dry and/or wet age related macular degeneration, AMD), post-operative cataract inflammation, uveitis (including posterior, anterior, intermediate and pan uveitis), iridocyclitis, scleritis, corneal graft and limbal cell transplant rejection, gluten sensitive enteropathy (coeliac disease), dermatitis herpetiformis, eosinophilic esophagitis, achalasia, autoimmune dysautonomia, autoimmune encephalomyelitis, autoimmune oophoritis, autoimmune orchitis, autoimmune pancreatitis, aortitis and periaortitis, autoimmune retinopathy, autoimmune urticaria, Behcet's disease, (idiopathic) Castleman's disease, Cogan's syndrome, IgG4-related disease, retroperitoneal fibrosis, juvenile idiopathic arthritis including systemic juvenile idiopathic arthritis (Still's disease), adult-onset Still's disease, ligneous conjunctivitis, Mooren's ulcer, pityriasis lichenoides et varioliformis acuta (PLEVA, also known as Mucha-Habermann disease), multifocal motor neuropathy (MMN), paediatric acute-onset neuropsychiatric syndrome (PANS) (including paediatric autoimmune neuropsychiatric disorders associated with streptococcal infections (PANDAS)), paraneoplastic syndromes (including paraneoplastic cerebellar degeneration, Lambert-Eaton myaesthenic syndrome, limbic encephalitis, brainstem encephalitis, opsoclonus myoclonus ataxia syndrome, anti-NMDA receptor encephalitis, thymoma-associated multiorgan autoimmunity), perivenous encephalomyelitis, reflex sympathetic dystrophy, relapsing polychondritis, sperm & testicular autoimmunity, Susac's syndrome, Tolosa-Hunt syndrome, Vogt-Koyanagi-Harada Disease, anti-synthetase syndrome, autoimmune enteropathy, immune dysregulation polyendocrinopathy enteropathy X-linked (IPEX), microscopic colitis, autoimmune lymphoproliferative syndrome (ALPS), autoimmune polyendocrinopathy-candidiasis-ectodermal dystrophy syndrome (APEX), gout, pseudogout, amyloid (including AA or secondary amyloidosis), eosinophilic fasciitis (Shulman syndrome) progesterone hypersensitivity (including progesterone dermatitis), amilial Mediterranean fever (FMF), tumour necrosis factor (TNF) receptor-associated periodic fever syndrome (TRAPS), hyperimmunoglobulinaemia D with periodic fever syndrome (HIDS), PAPA (pyogenic arthritis, pyoderma gangrenosum, severe cystic acne) syndrome, deficiency of interleukin-1 receptor antagonist (DIRA), deficiency of the interleukin-36-receptor antagonist (DITRA), cryopyrin-associated periodic syndromes (CAPS) (including familial cold autoinflammatory syndrome [FCAS], Muckle-Wells syndrome, neonatal onset multisystem inflammatory disease [NOMID]), NLRP12-associated autoinflammatory disorders (NLRP12AD), periodic fever aphthous stomatitis (PFAPA), chronic atypical neutrophilic dermatosis with lipodystrophy and elevated temperature (CANDLE), Majeed syndrome, Blau syndrome (also known as juvenile systemic granulomatosis), macrophage activation syndrome, chronic recurrent multifocal osteomyelitis (CRMO), familial cold autoinflammatory syndrome, mutant adenosine deaminase 2 and monogenic interferonopathies (including Aicardi-Goutières syndrome, retinal vasculopathy with cerebral leukodystrophy, spondyloenchondrodysplasia, STING [stimulator of interferon genes]-associated vasculopathy with onset in infancy, proteasome associated autoinflammatory syndromes, familial chilblain lupus, dyschromatosis symmetrica hereditaria), Schnitzler syndrome; familial cylindromatosis, congenital B cell lymphocytosis, OTULIN-related autoinflammatory syndrome, type 2 diabetes mellitus, insulin resistance and the metabolic syndrome (including obesity-associated inflammation), atherosclerotic disorders, and renal inflammatory disorders.

70-76. (canceled)

77. The method according to claim 64, wherein the compound or pharmaceutically acceptable salt and/or solvate thereof is administered in combination with a further therapeutic agent, selected from the group consisting of a corticosteroid (glucocorticoid), retinoid, anthralin, vitamin D analogue, calcineurin inhibitors, phototherapy or photochemotherapy or other form of ultraviolet light irradiation therapy, ciclosporine, a thiopurine, methotrexate, an anti-TNFα agents, phosphodiesterase-4 (PDE4) inhibition, anti-IL-17 agent, anti-IL12/IL-23 agent, anti-IL-23 agent, JAK (Janus Kinase) inhibitor, plasma exchange, intravenous immune globulin (IVIG), cyclophosphamide, anti-CD20 B cell depleting agent, anthracycline analogue, cladribine, sphingosine 1-phosphate receptor modulator or sphingosine analogue, interferon beta preparation (including interferon beta 1b/1a), glatiramer, anti-CD3 therapy, anti-CD52 targeting agent, leflunomide, teriflunomide, gold compound, laquinimod, potassium channel blocker, mycophenolic acid, mycophenolate mofetil, purine analogue, mTOR (mechanistic target of rapamycin) pathway inhibitor, antithymocyte globulin (ATG), IL-2 receptor (CD25) inhibitor, anti-IL-6 receptor or anti-IL-6 agent, Bruton's tyrosine kinase (BTK) inhibitor, tyrosine kinase inhibitor, ursodeoxycholic acid, hydroxychloroquine, chloroquine, B cell activating factor (BAFF, also known as BLyS, B lymphocyte stimulator) inhibitor, other B cell targeted therapy including a fusion protein targeting both APRIL (A PRoliferation-Inducing Ligand) and BLyS, PI3K inhibitor including pan-inhibitor or one targeting the p110δ and/or p110γ containing isoforms, an interferon α receptor inhibitor, T cell co-stimulation blocker, thalidomide and its derivatives, dapsone, clofazimine, a leukotriene antagonist, theophylline, anti-IgE therapy, an anti-IL-5 agent, a long-acting muscarinic agent, a PDE4 inhibitor, riluzole, a free radical scavenger, a proteasome inhibitor, a complement cascade inhibitor including one directed against C5, immunoadsor, antithymocyte globulin, 5-aminosalicylates and their derivatives, an anti-integrin agent including one targeting α4β1 and/or α4β7 integrins, an anti-CD11-α agent, a non-steroidal anti-inflammatory drug (NSAID) including a salicylate, a propionic acid, an acetic acid, an oxicam, a fenamate, a selective or relatively selective COX-2 inhibitor, colchicine, an IL-4 receptor inhibitor, topical/contact immunotherapy, anti-IL-1 receptor therapy, IL-1β inhibitor, IL-1 neutralising therapy, chlorambucil, a specific antibiotic with immunomodulatory properties and/or ability to modulate NRF2, anti-androgenic therapy, pentoxifylline, ursodeoxycholic acid, obeticholic acid, fibrate, a cystic fibrosis transmembrane conductance regulator (CFTR) modulator, a VEGF (vascular endothelial growth factor) inhibitor, pirfenidone and mizoribine.

78. A compound selected from the group consisting of:
(a) a compound of formula (II): 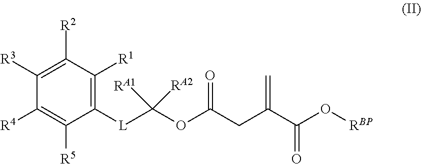
wherein R¹, R², R³, R⁴, R⁵, L, R^{A1}, R^{A2} and R^{B} are defined in claim 1, and R^{BP} is a protected derivative of R^{B};
or a salt thereof;
(b) a compound of formula (III): 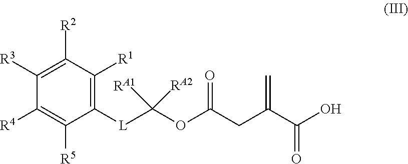
wherein R¹, R², R³, R⁴, R⁵, L, R^{A1} and R^{A2} are as defined in claim 1;
or a salt thereof;
(c) a compound of formula (IV): 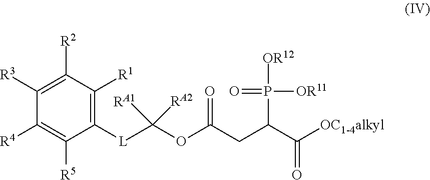
wherein R¹, R², R³, R⁴, R⁵, L, R^{A1} and R^{A2} are as defined in claim 1, and R¹¹ and R¹² independently represent C₁₋₄ alkyl optionally substituted with halo,
or a salt thereof;
(d) a compound of formula (VI): 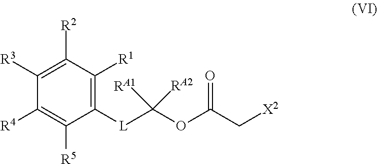
wherein R¹, R², R³, R⁴, R⁵, L, R^{A1} and R^{A2} are as defined in claim 1, and X² is a halo atom;
or a salt thereof; and
(e) a compound of formula (XVI): 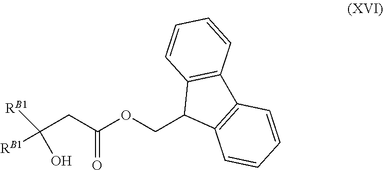
wherein R^{B1} is as defined in claim 1;
or a salt thereof.

79-94. (canceled)

95. A compound according to claim 78 or a salt thereof which is selected from the group consisting of:
4-(1-(3,5-dichlorophenyl)cyclobutyl) 1-methyl 2-(diethoxyphosphoryl)succinate;
4-(1-(3,5-dichlorophenyl)cyclobutyl) 1-methyl 2-methylenesuccinate;
4-(1-(3,5-dichlorophenyl)cyclobutoxy)-2-methylene-4-oxobutanoic acid; and
4-(1-(3,5-dichlorophenyl)cyclobutyl) 1-(2-oxo-2-(2,2,2-trichloroethoxy)ethyl) 2-methylenesuccinate.

96. (canceled)

97. A process for preparing a compound according to claim 1 or a salt thereof, which comprises reacting a compound of formula (III): 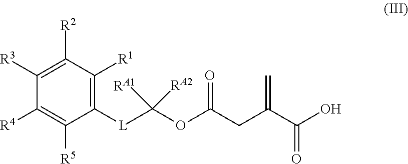
or a salt thereof,
with R^{BP}—X³
wherein R^{BP} is a protected derivative of R^{B}, and X³ is OH or a leaving group;
or a salt thereof;
followed by deprotection;
wherein R¹, R², R³, R⁴, R⁵, L, R^{A1}, R^{A2} and R^{B} are as defined in claim 1.

98-100. (canceled)
